(19) United States
(12) Patent Application Publication
(10) Pub. No.: US 2020/0116725 A1
(43) Pub. Date: Apr. 16, 2020

(54) RENAL CLEARABLE NANOCATALYSTS FOR DISEASE MONITORING

(71) Applicants: Massachusetts Institute of Technology, Cambridge, MA (US); President and Fellows of Harvard College, Cambridge, MA (US)

(72) Inventors: Sangeeta N. Bhatia, Lexington, MA (US); Molly Morag Stevens, London (GB); Colleen Loynachan, Somerville, MA (US); Ava Soleimany, Cambridge, MA (US)

(73) Assignees: Massachusetts Institute of Technology, Cambridge, MA (US); President and Fellows of Harvard College, Cambridge, MA (US)

(21) Appl. No.: 16/654,572

(22) Filed: Oct. 16, 2019

Related U.S. Application Data
(60) Provisional application No. 62/746,376, filed on Oct. 16, 2018.

(51) Int. Cl. G01N 33/574; G01N 21/76

(52) U.S. Cl. CPC G01N 33/57419; G01N 2333/435; G01N 21/76

(57) ABSTRACT

Aspects of the present disclosure relate to methods and compositions useful for in vivo and/or in vitro profiling of environmental triggers (e.g., enzyme activity, pH or temperature). In some embodiments, the disclosure provides methods of in vivo enzymatic processing of exogenous molecules followed by detection of nanocatalysts as representative of the presence of active enzymes (e.g., proteases) associated with a disease, for example, cancer or infection. In some embodiments, the disclosure provides compositions and methods for production of in vivo sensors comprising nanocatalysts.

### CROSS-REFERENCE TO RELATED APPLICATIONS

This application claims the benefit of priority under 35 U.S.C. § 119(e) of U.S. provisional application Ser. No. 62/746,376, filed Oct. 16, 2018, the disclosure of which is incorporated by reference here in its entirety.

### BACKGROUND

Early detection of disease often improves patient outcomes. For example, diagnosis when cancer is localized to the organ of origin correlates with significantly greater long-term survival as compared with when the cancer has metastasized. Currently available therapeutics are also often most effective during the early stages of disease. Furthermore, detection of an infectious disease (e.g., a bacterial or viral infection) prior to the onset of symptoms may facilitate the development of public health measures, such as containment and development of vaccines. Since each individual may differ in disease susceptibility due to genetics or may present with a heterogeneous disease, personalized medicine also benefits from early detection and monitoring of disease progression. Therefore, timely and accurate in vitro and in vivo diagnostic platforms are needed.

### SUMMARY

Aspects of the present disclosure provide an in vivo or in vitro sensor comprising a scaffold that is attached to an environmentally-responsive linker that is attached to a nanocatalyst, wherein the nanocatalyst is capable of being released from the scaffold when exposed to an environmental trigger. In some embodiments, the sensor is formulated for in vivo delivery. In some embodiments, the environmental trigger is an enzyme.

In certain embodiments, the scaffold encapsulates a nanocatalyst, optionally wherein the scaffold is a liposome, polymersome, or a PLGA nanoparticle.

In some embodiments, the nanocatalyst is a catalytic nanocluster or a nanocatalyst. In some embodiments, the catalytic nanocluster is a transition metal nanocluster selected from the group consisting of a platinum nanocluster, a silver nanocluster, and a gold nanocluster.

In some embodiments, the nanocatalyst is selected from the group consisting of an iron oxide nanoparticle and an iridium nanoparticle.

In some embodiments, the environmentally-responsive linker is temperature-responsive, pH-responsive, or an enzyme-specific substrate.

In some embodiments, the nanocatalyst is less than 5 nm in size, optionally less than 2 nm in size. In some embodiments, the scaffold is greater than about 5 nm in diameter.

In some embodiments, the scaffold comprises a protein, a polymer, or a nanoparticle. In some embodiments, the protein comprises avidin. In some embodiments, the avidin is selected from the group consisting of avidin, streptavidin, NeutrAvidin, and CaptAvidin.

In some embodiments, the environmentally-responsive linker is further attached to a functional handle and wherein the environmentally-responsive linker is located between the functional handle and the nanocatalyst. In some embodiments, the functional handle is selected from the group consisting of a dibenzocyclooctyne (DBCO), an amine, a SpyCatcher tag, a SpyTag, a biotin, avidin, an alkyne, and an azide.

In some embodiments, the functional handle is linked to the scaffold.

In some embodiments, the nanocatalyst is luminescent. In some embodiments, the nanocatalyst is capable of disproportionating H₂O₂. In some embodiments, the nanocatalyst is capable of disproportionating H₂O₂ in physiological environments. In some embodiments, the nanocatalyst comprises a zwitterionic peptide capping layer.

In some embodiments, the enzyme-specific substrate is a disease-specific substrate. In some embodiments, the disease is cancer, HIV, malaria, an infection or pulmonary embolism.

In some embodiments, the sensor comprises a single environmentally-responsive linker, a single nanocatalyst, or a combination thereof. In some embodiments, the sensor comprises multiple environmentally-responsive linkers, multiple nanoclusters, or a combination thereof. In some embodiments, the ratio of the number of environmentally-responsive linkers to the number of nanocatalysts is at least 1, optionally wherein the ratio is between 1 and 20.

In some embodiments, the surface area to volume ratio of the nanocatalyst is about 1.2 to about 6.

Another aspect of the present disclosure provides a method comprising:

(a) administering to a subject any of the sensors described herein,

(b) detecting in a biological sample obtained from the subject the nanocatalyst, wherein detection of the nanocatalyst in the biological sample is indicative of the environmental trigger being present within the subject. In some embodiments, the sensor comprises a scaffold comprising an environmentally-responsive linker that is attached to a nanocatalyst, wherein the nanocatalyst is capable of being released from the scaffold when exposed to an environmental trigger in vivo or in vitro. The subject may be a human subject.

In some embodiments, the nanocatalyst is a transition metal nanocluster, optionally, wherein the transition metal nanocluster is a platinum nanocluster, a silver nanocluster, or a gold nanocluster and optionally, wherein the nanocatalyst is an iron oxide nanoparticle, or an iridium nanoparticle,

In some embodiments, the environmentally-responsive linker is an enzyme-specific substrate, wherein the environmental trigger is the enzyme and wherein the detection of the nanocatalyst is indicative of the enzyme being in an active form within the subject. In some embodiments, the biological sample is not derived from the site of exposure to the environmental trigger, optionally wherein the sample is a urine sample, blood sample, or tissue sample.

In some embodiments, the detecting comprises a colorimetric assay, luminescence, or fluorescence assay.

In some embodiments, the detecting comprises detecting the catalytic activity of the nanocatalyst. In some embodiments, the detecting comprises an oxidation assay with a peroxidase substrate and detection of the oxidized substrate, optionally, wherein the peroxidase substrate is a chromogenic substrate.

In some embodiments, the enzyme-specific substrate is a disease-specific substrate.

In some embodiments, the method further comprises diagnosing the subject with the disease based on the detection of the nanocatalyst in the biological sample. In some embodiments, the disease is selected from the group consisting of cancer, HIV, malaria, an infection, and pulmonary embolism.

Another aspect of the present disclosure provides a method for producing one or more of the sensors described herein. The method may comprise incubating an environmentally-responsive linker and a reducing agent with chloroauric acid (HAuCl₄), wherein the environmentally-responsive linker comprises a cysteine residue or is thiol-terminated and wherein the resulting gold nanoclusters may be capped and stabilized by both the reducing agent and an environmentally-responsive linker and exhibit both intrinsic fluorescence and peroxidase-like catalytic activity, and wherein the gold nanocluster is capable of being released from the environmentally-responsive linker in vivo, optionally wherein the nanocluster synthesis proceeds at an elevated temperature of at least 70° C. for more than 12 hrs and optionally wherein the reducing agent is L-glutathione (GSH) peptide.

In some embodiments, the environmentally-responsive linker further comprises a functional handle.

In some embodiments, the functional handle is selected from the group consisting of a dibenzocyclooctyne (DBCO), an amine, a SpyCatcher tag, a SpyTag, a biotin, avidin, an alkyne, and an azide.

In some embodiments, the method further comprising incubating the environmentally-responsive linker attached to the nanocatalyst with a scaffold comprising a cognate functional handle partner, optionally wherein the cognate functional handle partner is selected from the group consisting of a dibenzocyclooctyne (DBCO), an amine, a SpyCatcher tag, a SpyTag, a biotin, an alkyne, and an azide.

In some embodiments, the avidin is selected from the group consisting of avidin, streptavidin, NeutrAvidin, and CaptAvidin.

In some embodiments, the gold nanocluster has a surface area to volume ratio of the gold nanocluster is about 1.2 to about 6. Another aspect of the present disclosure provides an in vivo or in vitro sensor comprising a scaffold that encapsulates nanocatalyst, wherein the nanocatalyst is capable of being released from the scaffold when exposed to an environmental trigger. In some embodiments, the sensor is formulated for in vivo delivery. In some embodiments, the environmental trigger is an enzyme.

In some embodiments, the scaffold is a liposome that comprises brain sphingomyelin (BSM) and cholesterol (CH).

In some embodiments, the scaffold is a liposome that comprises phosphatidylcholine (POPC).

In some embodiments, the environmental trigger is a phospholipase A2 (PLA2) enzyme, sphingomyelinase (SMase), and/or a toxin. In some embodiments, toxin is alpha-hemolysin.

Further aspects of the present disclosure provide methods comprising: (a) administering to a subject any of the sensors described herein, wherein the sensor comprises a scaffold that encapsulates a nanocatalyst, wherein the nanocatalyst is capable of being released from the scaffold when exposed to an environmental trigger in vivo or in vitro, optionally wherein the subject is a human subject; and (b) detecting in a biological sample obtained from the subject the nanocatalyst, wherein detection of the nanocatalyst in the biological sample is indicative of the environmental trigger being present within the subject.

### BRIEF DESCRIPTION OF DRAWINGS

FIGS. 1A-1C depict the design of a nanocatalyst signal amplification sensing system. FIG. 1A depicts catalytic gold nanoclusters (AuNCs) that were conjugated to an avidin protein scaffold through a biotinylated protease-cleavable peptide linker. FIG. 1B shows that the protease-sensitive nanocluster complex was injected intravenously and designed to specifically disassemble when exposed to the activity of relevant dysregulated proteases at the site of disease. After protease cleavage, liberated ca. 1.5 nm AuNCs were filtered into the urine. FIG. 1C shows that AuNCs were detected in cleared urine by measuring their ability to oxidize a chromogenic peroxidase substrate in the presence of hydrogen peroxide.

FIGS. 2A-2F depict that peptide-functionalized AuNCs exhibit stable catalytic activity. FIG. 2A is a schematic showing one-pot synthesis of AuNCs where thiol-terminated heterobifunctional peptides (P1₁₃, P1₂₀, P2₁₃, P2₂₀) are incorporated onto the AuNC surface. FIG. 2B is a transmission electron micrograph (TEM) of glutathione-protected AuNCs (GSH-AuNCs, scale=5 nm). The inset shows a high-resolution TEM of an individual GSH-AuNC (scale=2 nm). FIG. 2C is a histogram showing the results of a size analysis from TEM images (n≥200 particles). The solid line represents a Gaussian fit of size distribution. FIG. 2D is a graph showing the catalytic activity of AuNCs capped with different cysteine containing protease-cleavable peptide linkers (GSH, P1₁₃, P1₂₀, P2₁₃, P2₂₀, Table 3). Activity is measured by the absorbance at 652 nm corresponding to the oxidation of TMB by H202 and normalized here to the activity of GSH-AuNCs in PBS. FIG. 2E is a graph showing the limit of detection of reporter probes measured by catalytic activity of AuNCs functionalized with peptides P1_{13/20}, P2_{13/20}. Catalytic activity is measured by initial rate analysis (A_{652 nm}/s) of TMB oxidation. The solid line indicates that the activity for AuNCs is linear over 3 orders of magnitude of particle concentration. FIG. 2F is a graph depicting the catalytic activity of GSH-AuNCs, and representative AuNC-P1₂₀ batch incubated in serum and urine environments for 1 hour. Activity is normalized to activity of AuNCs in PBS.

FIGS. 3A-3C depict that peptide-functionalized AuNCs renally clear and retain their catalytic activity in urine. FIG. 3A is a schematic of the renal clearance assay. AuNCs were i.v. injected into Swiss Webster mice, and urine was collected 1 hour post-injection. Urine was analyzed by both TMB catalytic activity assay and by ICP-MS for gold. FIG. 3B is a graph showing renal clearance efficiency of GSH-AuNC, AuNC-P1₁₃, AuNC-P1₂₀, AuNC-P2₁₃, AuNC-P2₂₀ as measured by colorimetric assay (A_{652 nm}) and by ICP-MS (estimated ppb cleared), normalized to activity and gold content, respectively, of the injected dose (n≥3 per group). FIG. 3C shows the correlation between estimated renal clearance as measured by colorimetric activity assay and by ICP-MS (Pearson's r=0.49, *P<0.05).

FIGS. 4A-4F depict that AuNC-avidin complexes disassemble in vitro in response to protease activity. FIG. 4A is a schematic illustration of FCS measurement. First, AuNCs are labelled with fluorescent dye and complexed to neutravidin core. Dye-labelled AuNC-NAv complexes were incubated with relevant enzyme and FCS was used to monitor changes in diffusion time due to enzyme cleavage. FIG. 4B is a graph showing correlation curves from FCS measurements showing AuNC-P2₂₀-NAv complex in the presence of MMP9 over time compared to free AuNCs and Oregon green dye. A clear shift to smaller sizes is observed for longer enzyme incubation times (red to blue color change), indicating cleavage of AuNCs. FIG. 4C is a graph of hydrodynamic diameters extracted from FCS correlation curves showing changes in sizes of complexes after enzyme incubation. The dotted line represents renal filtration size cut-off of 5.5 nm. FIG. 4D is a plot of fraction of AuNCs liberated from AuNC-NAv complex for MMP9 responsive complexes composed of either short or long linker incubated with MMP9 up to 16 hours. Dotted line at 60 minutes is shown. This corresponds to the time frame of in vivo experiments. FIG. 4E shows catalytic activity of gel filtration chromatography (GFC) column fractions associated with AuNC-P1₂₀, AuNC-P1₂₀-NAv complex (Complex), 10 μM AuNC-P1₂₀-NAv incubated with 50 nM MMP9 for 12 h at 37° C. (Complex+MMP9), and 10 μM AuNC-P1₂₀-NAv complex incubated with 50 nM thrombin for 12 h at 37° C. (Complex +THR). FIG. 4F shows catalytic activity of GFC column fractions associated with AuNC-P2₂₀, AuNC-P2₂₀-NAv complex (Complex), 10 μM AuNC-P2₂₀-NAv complex incubated with 50 nM thrombin for 12 h at 37° C. (Complex+THR), and 10 μM AuNC-P2₂₀-NAv incubated with 50 nM MMP9 for 12 h at 37° C. (Complex+MMP9).

FIGS. 5A-5E depict that AuNC-functionalized protease nanosensors enable direct colorimetric urinary readout of disease state. FIG. 5A is a schematic showing that mice bearing LS174T flank xenografts and age-matched healthy controls were injected i.v. with AuNC-P2₂₀-NAv complex. Urine was collected 1 hour post injection, and renal clearance of liberated AuNCs was measured by catalytic activity assay. FIG. 5B is a photograph of representative examples of colorimetric assay on urine from tumor-bearing (top) and healthy (bottom) mice injected with AuNC-P2₂₀-NAv (n=4 mice per group shown). FIG. 5C is a graph showing the initial velocity of catalytic activity in urine collected from healthy and LS174T tumor bearing mice 1 hour after injection with AuNC-P2₂₀-NAv complex, as measured by the rate of change of A_{652 nm} over the first 10 minutes of the assay (n=8, **P<0.01, two-tail Student's t-test). FIG. 5D is a graph showing the receiver-operating characteristic (ROC) curve by initial velocity of catalytic activity assay discriminated healthy from diseased mice with an AUC of 0.95 (P=0.0023 from random classifier). FIG. 5E shows the results of a catalytic activity assay on urine from healthy and tumour-bearing mice injected with thrombin-responsive AuNC-P1₂₀-NAv complex. Inset: photograph of representative examples. No visible colorimetric development was observed in either group, and there was no statistically significant difference between the two groups (mean±s.d., n=8 mice per group, two-tailed Mann-Whitney test, ^{ns}P=0.161). Catalytic activity was measured by initial velocity analysis (A_{652 nm} min⁻¹), and dashed line represents limit of detection (see Methods).

FIGS. 6A-6F show proteolytic cleavage of peptide substrates. FIGS. 6A-6B show fluorescently quenched thrombin- or MMP-responsive (FIG. 6A and FIG. 6B, respectively) peptides were incubated with target enzyme. Proteolytic cleavage released the quencher, and fluorescence was measured to monitor kinetics. FIGS. 6C-6D show ICP-MS traces of thrombin-responsive P1₁₃ and P1₂₀ peptides (c and d, respectively) following incubation with recombinant thrombin. FIGS. 6E-6F show ICP-MS traces of MMP-responsive P2₁₃ and P2₂₀ peptides (FIG. 6C and FIG. 6D, respectively) following incubation with recombinant thrombin.

FIGS. 7A-7F show in vitro characterization of AuNCs. FIG. 7A shows catalytic activity of glutathione templated nanoclusters synthesized with varying core metals: gold, platinum and gold-platinum bimetallic hybrid. AuNCs exhibited the highest activity followed by Au—Pt with intermediate activity, and PtNCs showed the lowest of the tested metals. FIG. 7B shows AuNC synthesis showed high reproducibility with a coefficient of variation between seven independently synthesized batches of ca. 8.5%. The red line indicates the average test line intensity across batches. FIG. 7C shows UV-vis absorption spectrum of peptide templated AuNC batches compared to 40 nm AuNP. AuNCs do not exhibit surface plasmon resonance peak at 520 nm characteristic of large gold nanoparticles. FIG. 7D shows fluorescence excitation (Em: 600 nm, dotted line) and emission (Ex. 400 nm, solid line) spectrum. FIG. 7E shows the structure of 3,3′,5,5′-tetramethybenzidine (TMB) (i), and oxidized TMB (TMB diimine) (ii). FIG. 7F is a representative UV/vis spectra showing increase in absorbance correlated to oxidation of TMB in presence of varying concentrations of AuNCs, where the experiment was repeated independently 3 times with similar results. An increase in absorbance at both 370 nm and 652 nm was observed for increasing concentration of nanocatalyst with fixed concentrations of TMB and H₂O₂ substrates. Inset shows photo of substrate alone (left) and substrate with AuNCs showing colour development (right).

FIGS. 8A-8H show representative TEM images of AuNCs synthesized with different peptide sequences and corresponding size analysis. FIGS. 8A-8B show AuNC-P1₁₃, FIGS. 8C-8D showAuNC-P1₂₀, FIGS. 8E-8F showAuNC-P2₁₃, FIGS. 8G-8H showAuNC-P2₂₀. Scale bars, 5 nm.

FIGS. 9A-9D show number size distribution measured by dynamic light scattering (DLS) of AuNCs synthesized with different peptide sequences FIG. 9A shows P1₁₃, FIG. 9B shows P1₂₀, FIG. 9C shows P2₁₃, FIG. 9D shows P2₂₀. Increasing intensity of colored line corresponds to increasing concentration of protease-cleavable peptide sequence in synthesis. P1₁₃1:9 corresponds to a 1:9 ratio of P1₁₃ peptide: glutathione ratio in synthesis. All particles are synthesized with a fixed peptide concentration.

FIGS. 10A-10F show characterization of activity assay conditions. FIG. 10A shows catalytic activity of GSH-AuNCs as a function of hydrogen peroxide concentration. Activity is measured by the absorbance at 652 nm corresponding to the oxidation of TMB by H₂O₂. FIG. 10B shows catalytic activity of GSH-AuNCs as a function of pH. FIG. 10C shows kinetic measurement of catalytic activity with varying sodium chloride concentration (gray no salt, 0.01, 0.02, 0.05, 0.1, 0.2, 0.5, 1 M NaCl increasing color intensity), where precipitation of substrate occurred at high [NaCl]. FIG. 10D shows catalytic activity with varying [NaCl] after two minutes development (dotted line in FIG. 10C). FIGS. 10E-10F show steady-state kinetic assays of GSH-AuNCs as catalysts for the oxidation of TMB by H₂O₂. The initial reaction velocity (v) was measured in 25 mM sodium acetate buffer pH 4.0 with 1.8×10⁻⁶M AuNCs at room temperature over 150 seconds. Error bars indicate standard deviation of three independent measurements. FIG. 10E shows a plot of v against H₂O₂ concentration, in which TMB concentration was fixed at 0.45 mM. The apparent Kₘ value of the GSH-AuNCs with H₂O₂ as the substrate was significantly higher than that for HRP, consistent with the observation that a higher concentration of H₂O₂ was required to observe maximal activity for the AuNCs. FIG. 1OF shows a plot of v against TMB concentration, in which H₂O₂ was fixed at 2.3 M.

FIGS. 11A-11D show peptide incorporation and functionalization of AuNCs. FIGS. 11A-11B show catalytic activity of AuNCs synthesized with varying peptide sequences (P1₁₃, P1₂₀, P2₁₃, P2₂₀) and varying ratios of protease-cleavable peptide sequence to glutathione (1:9, 1:5, 1:4, 1:2), where activity is normalized to activity of AuNCs synthesized in the absence of P1_{13/20}, P2_{13/20} (glutathione only, GSH-AuNCs). FIG. 11C shows quantification of biotin ligands per AuNC when ratio of peptide sequence (P1_{13/20}, P2_{13/20}) to glutathione was varied in the synthesis. Dotted line represents estimated maximum number of ligands per AuNCs assuming ca. 100 atom AuNC (Au₁₀₂(SR)₄₄) (Jung et al., Nanoscale 2012, 4, 4206). Amount of biotin in supernatant of AuNC synthesis after purification was measured using 4′-hydroxyazobenzene-2-carboxylic acid (HABA)-avidin reagents, and biotin concentration on the particles was extrapolated using the starting concentration of biotin in synthesis and estimated concentration of AuNCs. FIG. 11D shows the functional performance of the AuNC batches containing different ratios of the protease cleavable substrates on the surface was tested using a paper-based assay. The assay used a streptavidin test line to measure the ability of the AuNC to effectively bind to avidin and a subsequent catalytic development step to probe the activity of the particles. It was found that there was an optimal ratio of protease substrate incorporated in the synthesis which led to efficient capping of the gold core with biotinylated protease cleavable ligands while retaining activity to preserve diagnostic sensitivity (1:5 ratio for thrombin substrates (P1) and a 1:4 ratio for MMP substrates (P2), which is taken forward in synthesis of particles in the following figures). The optimal substrate incorporation for efficient synthesis corresponds to ca. 15-20 biotinylated protease substrates per AuNC. Test line intensity quantified in ImageJ corresponding to AuNC-P1₁₃/P1₂₀ binding to polystreptavidin test line. AuNCs bound at the test line catalyze the oxidation of CN/DAB (4-chloro-1-naphthol/3,3′-diaminobenzidine tetrahydrochloride) substrate in the presence of hydrogen peroxide producing an insoluble black product.

FIGS. 12A-12B show assessment of endogenous peroxidase activity in mouse urine. FIG. 12A shows kinetic measurement of catalytic activity in urine of mice injected with GSH-AuNCs or PBS. FIG. 12B shows quantification of initial velocity of catalytic activity from FIG. 12A, as measured by the rate of change of A652 nm over the first 10 minutes of the reaction.

FIGS. 13A-13F show synthesis efficiency, stability, and size characterization of AuNC-Avidin complexes. FIG. 13A shows quantification of efficiency of binding of biotinylated AuNCs to neutravidin protein for varying neutravidin concentrations, where 4 mg.mL⁻¹ represents a 1.2 molar excess of AuNCs to avidin, and 0.5 mg.mL⁻¹ represents a 9.6 molar excess of AuNCs to avidin. Loading efficiency was measured by calculating the difference in catalytic activity of AuNC-Avidin before and after ultrafiltration purification to remove unbound AuNCs. Incubation with higher concentrations of avidin increased the efficiency of complex formation. FIG. 13B shows catalytic activity of AuNCs and AuNC complex after incubation in urine or fetal bovine serum (FBS) for 1 h. Activity is normalized to activity of sample in PBS. FIGS. 13C-13F show number distribution of hydrodynamic diameter measured by DLS for AuNC-P1₁₃, -P1₂₀, -P2₁₃, -P2₂₀ and corresponding AuNC-avidin complexes prepared with each particle batch after purification.

FIGS. 14A-14F show gel filtration chromatography (GFC) setup for measuring AuNC-avidin complex dissociation. FIG. 14A shows Number distribution of the hydrodynamic diameter of AuNC-P1₂₀, neutravidin, and AuNC-avidin complex (Complex) measured by dynamic light scattering (DLS). FIG. 14B shows a schematic of in vitro assay to monitor size of AuNC-avidin complex in response to recombinant protease activity. The schematic on the left shows that gel filtration chromatography is used to separate molecules based on size (free AuNCs are smaller than AuNC-avidin complex). The schematic on the top right shows that catalytic activity assay is performed on collected column fractions. The schematic on the bottom right shows that the activity of column fractions can be plotted against eluted volume and area under curve can be used to determine ratio of free AuNCs to complex. FIG. 14C shows AuNC-P1₁₃, AuNC-P1₁₃-NAv complex (Complex), and 10 μM AuNC-P1₁₃-NAv complex incubated with 50 nM thrombin for 12 h at 37° C. (Complex+THR). FIG. 14D shows AuNC-P2₁₃, AuNC-P2₁₃-NAv complex (Complex), and 10 μM AuNC-P2₁₃-NAv complex incubated with 50 nM MMP9 for 12 h at 37° C. (Complex+MMP9). FIG. 14E shows the activity of GFC column fractions for AuNC-NAv complexes prepared with different P1₂₀ loadings (P1₂₀:GSH 1:5 or 1:20), where 1:5 case has ca. 20 biotin ligands per AuNC and 1:20 case has ca. 5 biotin ligands per AuNC, for 1 h incubation with 50 nM thrombin at 37° C. FIG. 14F shows AuNC-P2₂₀-NAv complex incubated with 50 nM MMPI, MMP9, and MMP13 for 12 h at 37° C.

FIGS. 15A-15B show pharmacokinetic characterization of neutravidin protein carrier. FIG. 15A shows plasma concentration of fluorescently labeled neutravidin protein carrier was fit to a one-phase exponential decay. FIG. 15B shows Organs and tumors were harvested 1 hour after intravenous injection of fluorescently labeled neutravidin, and accumulation was measured by an IR scanner.

FIGS. 16A-16C show verification of colorimetric disease detection in LS174T tumor model. FIG. 16A shows a graph of catalytic activity assay on urine from healthy and tumor-bearing mice injected with PBS (n=3 mice per group). No colorimetric development was observed in either group, and there was no statistically significant difference between the two groups (two-tailed Student's t-test). FIG. 16B shows a graph of catalytic activity assay on urine from healthy and tumor-bearing mice injected with thrombin-responsive AuNC-P1₂₀-NAv complex (n=8 mice per group). No colorimetric development was observed in either group, and there was no statistically significant difference between the two groups (two-tailed Student's t-test). FIG. 16C shows collected urine volumes for samples used in colorimetric disease detection experiment (FIG. 5, n=8). No statistically significant difference in urine volume was observed between the two groups (two-tailed Student's t-test).

FIGS. 17A-17C show Biocompatibility of AuNC-avidin nanosensor complex. FIG. 17A shows In vitro cytotoxicity of AuNC-avidin nanosensor complex towards HEK293T cells, determined by the MTT assay. AuNC-P2₂₀-NAv at the indicated concentrations were incubated with cells for 24 h. FIG. 17B shows change in body mass of immunocompetent Swiss Webster mice injected with AuNC-P1₂₀-NAv (n=4, dose=3000 pmol) compared with PBS control (n=4). There is no statistically significant difference in the mass change between control and AuNC-avidin complex over a period of 4 weeks. FIG. 17C shows kidney, liver, and spleen histology. Organs were collected from mice 4 weeks after intravenous injection of

AuNC-P1₂₀-NAv or PBS into immunocompetent Swiss Webster mice. Organs were fixed, embedded in paraffin, and stained with haematoxylin and eosin. Analysis by a veterinary pathologist confirmed that tissue from AuNC-P1₂₀-NAv injected animals appeared similar to control animals. Study was done with n=4 mice and images from a representative animal are shown. Scale bars represent 100 μM.

FIGS. 18A-18B show hydrodynamic diameters calculated from FCS autocorrelation curves showing sizes of Oregon Green (OG) fluorescent dye, AuNC-NAv complexes, and AuNCs after incubation in PBS (black) or physiological environments (red or yellow). FIG. 18A shows the results with AuNC-P2₂₀-NAv complex incubated in PBS (black) or 10% v/v fetal bovine serum (FBS, red) for 1 h and 4 h (one-way ANOVA with Dunnett's multiple comparison, nsP=0.187 for 1 h, ^{ns}P=0.382 for 4 h). FIG. 18B shows the results with AuNC-P2₂₀ incubated in PBS (black) or undiluted synthetic urine (yellow) for 1 h and 26 h (one-way ANOVA with Dunnett's multiple comparison, ^{ns}P=0.470 for 1 h, ^{ns}P=0.657 for 26 h). Dashed line represents renal filtration size cut-off of ca. 5 nm. Individual sample measurements are represented as open circles with overlaid mean and standard deviation (n=25 independent measurements).

FIGS. 19A-19D show characterization of AuNCs in urine after kidney filtration. FIG. 19A includes a histogram showing results of size analysis from TEM images of GSH-AuNCs (legend shows mean diameter±s.d., n=167 particles) in mouse urine that was collected 1 h p.i. with AuNC samples. FIG. 19B includes a histogram showing results of size analysis from TEM images of AuNC-P2₂₀ (n=209 particles) in mouse urine that was collected 1 h p.i. with AuNC samples. FIG. 19C shows AuNCs in mouse urine 1 h p.i. of MMP9-responsive AuNC-P2₂₀-NAv complexes in tumour-bearing mice, indicating successful cleavage and renal elimination of liberated AuNCs in tumour model (n=449 particles). Solid line represents Gaussian fit of size distribution. Inset shows representative TEM images used for size analysis for each particle batch (scale=5 nm). AuNC samples in urine were desalted and purified through centrifugal ultrafiltration prior to imaging. FIG. 19D shows Energy Dispersive X-ray (EDS) point spectra analysis of the elemental composition of randomly selected areas across TEM grids containing cleared GSH-AuNCs in urine, where the experiment was repeated independently 3 times with similar results. EDS spectrum confirms the presence of gold and other elements that may be excreted by the kidneys, including calcium and magnesium, in addition to copper, carbon, and silicon signal from the TEM grid. Inset shows representative TEM image of grid area used for EDS analysis showing lattice fringes on renally cleared AuNCs (scale=5 nm).

FIGS. 20A-20B show stability of AuNCs in presence of physiological glutathione concentrations. FIG. 20A shows catalytic activity of GSH-AuNCs incubated with excess glutathione up to 2.5 mM for 1 h at 37° C. The dashed line indicates the average catalytic activity measured as absorbance at 652 nm corresponding to the oxidation of TMB across all samples analysed (mean±s.d., n=3 independent experiments). FIG. 20B shows number particle size distribution (PSD) measured by DLS of GSH-AuNCs in PBS and GSH-AuNCs incubated in the presence of 1 mM glutathione for 1 h at 37° C., where the DLS experiment was repeated independently 3 times with similar results.

FIGS. 21A-21B show cleavage kinetics of thrombin-responsive nanosensor. FIG. 21A shows average autocorrelation curves from FCS measurements (n=25 independent measurements) showing AuNC-P1₂₀-NAv complex in the presence of thrombin over time compared to free labelled AuNCs and Oregon Green dye (dashed lines: experimental; solid lines: fits). The curves from left to right along the x-axis correspond to results with Oregon Green, AuNC-P1₂₀ and THR 16 min (overlapping), THR 1 min, and AuNC-P1₂₀-NAv. A clear shift to faster diffusion times was observed for longer enzyme incubation times where the complex incubated with thrombin for 16 min overlaps with the AuNC-P1₂₀ curve, indicating complete cleavage of AuNCs from the complex in this timeframe. FIG. 21B shows plot of fraction of AuNCs liberated (see FCS in Methods of Example 8) from AuNC-P1₂₀-NAv complex incubated with thrombin (50 nM) up to 45 min. (mean±s.d., n=25 independent measurements).

FIGS. 22A-22B show results of probing MMP9 in vitro limit of detection. FIG. 22A shows a plot of fraction of AuNCs liberated (see FCS in Methods of Example 8) from AuNC-P2₂₀-NAv (15 μM) incubated with varying concentrations of MMP9 (2.5-50 nM) for 1 h to mimic in vivo experimental time frame (mean±s.d., n=25 independent measurements). To assemble the complexes for FCS analysis, the AuNC-P2₂₀ were first labelled with Oregon Green (OG_{488 nm}) dye prior to forming a complex with neutravidin. Dashed line represents mean of background signal (samples spiked with PBS instead of MMP9). FIG. 22B shows a plot of absorbance (proportional to catalytic activity of AuNCs) of filtrate containing liberated AuNCs after incubation of AuNC-P2₂₀-NAv (15 μM) with varying concentrations of MMP9 (0.2-100 nM) for 1 h to mimic in vivo experimental time frame (mean±s.d., n=3 independent experiments) and separated using 50 kDa cut-off centrifugal filter (pore size ca. 5 nm). Dashed line represents the detection cut-off calculated as 3 standard deviations above the mean of the background signal (samples spiked with PBS instead of MMP9).

FIGS. 23A-23C show biocompatibility of AuNC-NAv complex. FIG. 23A show in vitro cytotoxicity of AuNC-NAv complex towards HEK293T cells, determined by the MTS assay (mean±s.d., n=3 biologically independent samples). AuNC-P2₂₀-NAv at the indicated concentrations was incubated with cells for 24 h. FIG. 23B shows change in body mass of immunocompetent Swiss Webster mice injected with AuNC-P1₂₀-NAv (dose=3000 pmol, 200 μl of 15 μM [AuNC]) compared with PBS control (mean±s.d., n=4 mice per group). There was no statistically significant difference in the mass change over a period of 4 weeks between control mice (PBS injection) and mice injected with AuNC-NAv complex (multiple t-tests with Holm-Sidak correction for multiple comparisons; ^{ns}P=0.936 for 0, 11, 21, and 28 d; ^{ns}P=0.887 for 5 d). FIG. 23C shows results with immunocompetent Swiss Webster mice that were i.v. injected with AuNC-P2₂₀-NAv (dose=3000 pmol, 200 μL of 15 μM [AuNC]) and organs (heart, lung, liver, spleen, and kidney) that were collected at 1 h, 24 h, and 10 days post administration. Organs were fixed, embedded in paraffin, and stained with hematoxylin & eosin. Analysis by a veterinary pathologist confirmed that tissues from AuNC-NAv injected animals appeared similar to PBS injected controls, exhibiting no signs of toxicity. Study was done with n=3 mice per group and images from representative animals are shown. Scale bar represents 200 μm.

FIGS. 24A-24H show organ biodistribution and renal clearance of AuNCs in healthy mice. FIG. 24A is a schematic of the biodistribution and renal clearance study. Near-IR dye labelled GSH-AuNCs were i.v. injected into mice (10 μM, 200 μL), and urine samples were collected, and major organs harvested at time points up to 7 days p.i. FIG. 24B includes results with either IR labelled GSH-AuNCs (GSH-AuNC-IR) or unlabelled GSH-AuNCs were i.v. injected into Swiss Webster mice, and urine was collected 1 h post-injection. Urine was analysed by both TMB catalytic activity assay and by ICP-MS to measure gold content, where both techniques corroborated ca. 47% AuNC clearance compared to the injected dose at 1 h (mean±s.d., n=4 mice). FIGS. 24C-24F show the results of organs that were harvested at different times. Organs were harvested at 1 h in FIG. 24C, 3 h in FIG. 24D, 24 h in FIG. 24E, and 1 week in FIG. 24F after i.v. injection (10 μM, 200 μL) of near IR-dye labelled GSH-AuNCs into Swiss Webster mice, and the signal intensity in each organ was measured by an Odyssey IR scanner (mean±s.d., n=4 mice). Organ accumulation (y-axis) is presented as signal intensity per unit area, calculated for each organ as the difference between the experimental group (near IR-dye labelled GSH-AuNCs) versus the PBS-injected control. GSH-AuNCs accumulated significantly in kidneys 1 h post i.v. administration (one-way ANOVA with Tukey's multiple comparison test, ****P<0.0001). Kidney accumulation was significantly reduced 1 week post administration of GSH-AuNCs, likely due to excretion of AuNCs into urine. FIG. 24G shows a renal clearance time course of IR labelled GSH-AuNC or unlabelled GSH-AuNC in collected urine as measured by ICP-MS (estimated ppb cleared), normalized to gold content of the injected dose (mean±s.d., n=4 mice). Gold content was below the limit of detection in urine after 24 h p.i., where the detection cut-off was calculated as 3 standard deviations above the mean gold signal from PBS injected control mice (cut-off=0.13% ID). FIG. 24H shows kidney accumulation from biodistribution time course monitored up to 1 week p.i. (normalized to 1 h). AuNC signal was undetectable in kidneys at 1 week p.i. (mean±s.d., n=4 mice).

FIGS. 25A-25F include data showing a time course biodistribution of AuNC-NAv complex in healthy mice. FIG. 25A is a schematic of the biodistribution and pharmacokinetics study, where IR-dye labelled AuNC-P2₂₀-NAv complexes were i.v. injected into Swiss Webster mice (15 μM, 200 μL), and blood samples were collected, and major organs harvested at time points up to 4 weeks p.i. FIG. 25B shows pharmacokinetic characterization of IR-dye labelled AuNC-P2₂₀-NAv complex in Swiss Webster mice. Plasma concentration of nanosensor was fit to a two-phase exponential decay (mean±s.d., n=5 mice). FIGS. 25C-25F show results with organs that were harvested at various times. Organs were harvested at 1 h in FIG. 25C, 24 h in FIG. 25D, 1 week in FIG. 25E, and 4 weeks in

FIG. 25F after i.v. injection (15 μM, 200 μL) of near IR-dye labelled AuNC-P2₂₀-NAv complex into healthy Swiss Webster mice, and the signal intensity in each organ was measured by an Odyssey IR scanner (mean±s.d., n=4 mice). Organ accumulation (y-axis) is presented as signal intensity per unit area, calculated for each organ as the difference between the experimental group (fluorescently labelled AuNC-NAv complex) versus the PBS-injected control. AuNC signal was maximum at 1 h for all organs except for the liver and was undetectable in all organs at 4 weeks p.i.

FIGS. 26A-26C show entry of AuNC nanosensor complexes into tumours at 1 h p.i. Organs and tumours were harvested 1 h after i.v. injection of near IR-dye labelled neutravidin carrier (FIG. 26A), MMP-cleavable AuNC-P2₂₀-NAv complex (FIG. 26B), where signal arises from contribution of both liberated AuNCs and intact AuNC-NAv complex, or free AuNCs (FIG. 26C), into LS174T tumour-bearing mice, and the signal intensity in each organ was measured by an Odyssey IR scanner (mean±s.d., FIG. 26A, FIG. 26C n=4 mice; FIG. 26B n=5 mice). Organ accumulation (y-axis) is presented as signal intensity per unit area, calculated for each organ as the difference between the experimental group (fluorescently labelled carrier, complex, or nanocluster) versus the PBS-injected control.

FIGS. 27A-27B show a non-limiting example of a liposome encapsulated nanocatalysts for sensing of disease-associated enzymes. FIG. 27A shows a liposome platform to encapsulate nanocatalysts in aqueous core. Liposomes are ruptured upon interaction with disease-associated enzymes (e.g. sphingomyelinase and bacterial pore-forming toxins). FIG. 27B shows the results of a catalytic activity assay to measure presence of liberated/unencapsulated nanocatalysts in representative liposome samples pre-enzyme incubation and post-enzyme incubation. Enzyme incubation results in ruptured liposomes, and liberated nanocatalysts that produce blue colored signal upon interaction with H₂O₂ and peroxidase substrate tetramethylbenzidine. Liposome formulations tested included phosphatidylcholine (POPC), specifically disrupted by the enzyme phospholipase A2 (PLA₂), and brain sphingomyelin:cholesterol (BSM:CH, 50:50 w:w), specifically disrupted by the enzyme sphingomyelinase (SMase) and other pore-forming bacterial toxins (e.g. alpha hemolysin).

FIGS. 28A-28B include data showing that AuNC-functionalized protease nanosensors enable a direct colorimetric urinary readout of the disease state. FIG. 28A shows the results of a catalytic activity assay on urine collected from healthy and LS174T tumour-bearing mice 1 h p.i. with the AuNC-P2₂₀-NAv complex (mean±s.d., N=2 independent experiments indicated in shades, n=6 (lighter data points for each type of mice) or 8 (darker data points for each type of mice) mice per group, two-tailed Mann-Whitney test, ***P=0.0002). The catalytic activity was measured by initial velocity analysis (A652 min⁻¹), and the dashed line represents the LoD (Methods in Example 8). FIG. 28B shows that a receiver operating characteristic curve by the initial velocity of the catalytic activity assay discriminated healthy from diseased mice with an area under the curve of 0.91 (N=2 independent experiments, n=6 or 8 mice per group as in FIG. 28A, P=0.0002 from a random classifier shown by the dashed line).

### DETAILED DESCRIPTION

Aspects of the disclosure relate to in vitro and in vivo sensors comprising nanocatalysts for detecting and monitoring environmental triggers within a disease microenvironment as an indicator of certain disease states (e.g., presence of a disease, type of disease, severity of a disease, etc.). As described below, environmental triggers associated with disease include enzyme (e.g., protease) activity, pH, light, and temperature. The disclosure relates, in some aspects, to the surprising discovery that small transition metal nanoparticles, (e.g., nanoclusters comprising several to a few hundred atoms), including gold nanocluster (AuNC)-functionalized protease nanosensors can be used to provide an affordable, sensitive, and rapid colorimetric urinary readout in diseases such as cancer and pulmonary embolism.

The continuing hurdle of developing PoC diagnostics is that often compromises must be made between sensitivity, simplicity, speed, and cost. Dysregulated protease activities are implicated in a wide range of human diseases; including cancer, inflammation, and infectious diseases such as HIV and malaria. The ability to monitor protease activities in vivo with a simple and sensitive readout may enable earlier detection and monitoring of disease in resource-limited or home settings (Dudani et al., Annu. Rev. Cancer Biol. 2, 53-76 (2018)). Democratization of diagnostic tools to enable simple, sensitive, and early detection of disease is essential, particularly in low- and middle-income countries, which bear a significant burden of both infectious and noncommunicable diseases (World Health Organization. Global action plan for the prevention and control of noncommunicable diseases 2013-2020. (2013)). While worldwide mortality rates due to infectious diseases have substantially decreased, the ever increasing ageing population means cancer has become a primary cause of morbidity and mortality (Selmouni et al., Lancet Oncol. 19, e93-e101 (2018)).

Early diagnosis of cancer enables effective treatment of primary tumours via local therapeutic interventions such as surgery and radiotherapy (Etzioni et al., Nat. Rev. Cancer 3, 235 (2003)). Early detection has largely relied on blood biomarkers. However, the prohibitively low rates that most biomarkers are shed from tumours, the tremendous dilution into circulation, and the lack of specificity of secreted biomarkers impede early detection (Hori et al., Sci. Transl. Med. 3, 109ra116 (2011); Herny et al., Mol. Oncol. 6, 140-146 (2012)). Protease activities are implicated in a wide range of noncommunicable human diseases including cancer, inflammation, and thrombosis. Monitoring protease activity as a biomarker of disease may be leveraged to overcome the lack of sensitivity and specificity of abundance-based blood biomarkers (Lopez-Otin et al., J. Biol. Chem. 283, 30433-30437 (2008)). Common tools to measure protease activity often rely on cumbersome and infrastructure heavy analyses, such as fluorescence (Hilderbrand et al., Curr. Opin. Chem. Biol. 14, 71-79 (2010); Whitney et al., Angew. Chemie—Int. Ed. 52, 325-330 (2013); Whitley et al., Sci. Transl. Med. 8, (2016)), mass spectrometry (Yepes et al., Proteomics—Clin. Appl. 8, 308-316 (2014)), or MRI (Choi et al., Nat. Mater. 16, 537-542 (2017)). Previously, we developed exogenously administered multiplexed protease-responsive nanoparticles that release small reporter probes into the urine in response to proteolytic cleavage in disease environments (Kwon et al., Proc. Natl. Acad. Sci. 112, 14460-14466 (2015); Warren et al., Proc. Natl. Acad. Sci. U. S. A. 111, 3671-6 (2014); Kwon et al., Nat. Biomed. Eng. 1, 0054 (2017); Shuerle et al., Nano Lett. 16, 6303-6310 (2016)). For precision medicine to become globally accessible, diagnostic tools that can probe protease activity with a simple and sensitive readout are required.

Although gold nanoclusters (AuNCs) have recently been used for fluorescence and x-ray contrast bioimaging applications (Zhang et al., Sci. Rep. 5, 8669 (2015); Chen et al., Nano Lett. 17, 6330-6334 (2017)), the catalytic activity (e.g., surface catalytic activity) of these nanoclusters has yet to be explored for in vivo biosensing. Without being bound by a particular theory, the ultra-small size of AuNCs (<2 nm) induces quantum confinement effects, which result in discrete electronic and molecular-like properties, such as enhanced photoluminescence, intrinsic magnetism, and catalytic activity. In some aspects of the present disclosure, transition metal nanoparticles and nanoclusters are used as catalysts to disproportionate H₂O₂, which in turn can oxidize a chromogenic substrate, providing a colorimetric measure of activity, similar to the biological enzyme horseradish peroxidase (HRP). Employing peroxidase-mimicking catalytic AuNCs as reporter probes in sensing applications may enable rapid and facile disease diagnosis in low-infrastructure settings and at the point-of-care, where equipment and personnel may be limited.

As described herein, a modular approach has been developed for rapid detection of a disease state based on a simple and sensitive colorimetric urinary assay that requires minimal equipment and can be read by eye in, for example, <1 h. ca. 2 nm catalytic gold nanocluster probes modified with orthogonal protease substrates were synthesized, which are responsive to multiple enzymes. As demonstrated herein, the peptide-templated AuNCs could be filtered through the kidneys and excreted into the urine with high efficiency and retain catalytic activity in complex physiological environments. The AuNC probes were assembled into larger complexes, which were disassembled in response to specific proteases. Finally, in some embodiments, MMP-responsive AuNC-NAv complexes were deployed in vivo in a colorectal cancer mouse model and successfully detected AuNCs in urine from tumour-bearing mice with a facile colorimetric readout. Surprisingly, it was shown that AuNCs are small enough to be filtered efficiently through the kidneys and retain catalytic activity in cleared urine, thus providing a versatile disease detection platform that is compatible for deployment at the point-of-care (PoC).

A versatile toolbox is presented herein that can be used to probe the complex enzymatic profiles of specific disease microenvironments, the results of which will open new opportunities for developing translatable responsive and catalytic nanomaterial diagnostics for a range of diseases in which enzyme activity can be used as a biomarker. In some embodiments, clinical application of this technology may additionally take advantage of multiplexed protease substrate linkages, such as those responsive to Boolean logic operations (Von Maltzahn et al., J. Am. Chem. Soc. 129, 6064-6065 (2007); Badeau et al., Nat. Chem. 10, 251-258 (2018)), which may be able to profile the activities of proteases of diverse classes in order to distinguish between cancers and other pathologies. The adaptable nanocatalyst amplification platform described herein may be applicable in low-resource settings for rapid detection of a diverse range of disease-associated proteases, including those implicated in infectious diseases, and will democratize access to advanced and sensitive diagnostics.

Accordingly, provided herein, in some embodiments, are in vivo sensors comprising a scaffold comprising an environmentally-responsive linker that is attached to a nanocatalyst. The nanocatalyst is capable of being released from the sensor when exposed to an environmental trigger.

The sensors of the present disclosure comprise a modular structure having a scaffold linked to an environmentally-responsive linker that is attached to a nanocatalyst. As used herein, a nanocatalyst is a nanoparticle exhibiting catalytic activity. Non-limiting examples of nanocatalysts include catalytic nanoclusters (e.g., nanocatalysts with less than 2 nm in diameter). In some embodiments, a nanocluster comprises at most 500 atoms (e.g., at most 400, at most 300, at most 200, at most 100, at most 50, at most 25, at most 10, or at most 5 atoms). In some embodiments, a nanocluster comprises one or more transition metals (e.g., gold, platinum, gold-platinum, bimetallic, iron, palladium, iridium, or any combination thereof).

A modular structure, as used herein, refers to a molecule having multiple domains. The sensor, alternatively referred to as a nanosensor, when exposed to an environmental trigger will be modified such that the nanocatalyst is released from the scaffold.

The scaffold may include a single type of environmentally-responsive linker, such as a substrate (e.g., one or more substrates of the same enzyme), a pH-sensitive linker, or temperature-sensitive linker. The scaffold may include multiple types of different environmentally-responsive linkers (e.g., a pH-sensitive linker, a temperature-sensitive linker, and/or an enzyme substrate). For instance each scaffold may include a single (e.g., 1) type of environmentally-responsive linker or it may include 2-1,000 different environmentally-responsive linkers, or any integer therebetween. Alternatively, each scaffold may include greater than 1,000 different environmentally-responsive linkers. Multiple copies of the sensors are administered to the subject. In some embodiments, a composition comprising a plurality of different sensors (e.g. protease nanosensors) may be administered to a subject to determine whether multiple enzymes and/or substrates are present. In that instance, the plurality of different sensors may include one or more nanocatalysts.

In some embodiments, the ratio of the number of environmentally-responsive linkers to the number of catalytic nanoclusters is at least 0.5 (e.g., at least 1, at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30 , at least 40, at least 50, at least 60, at least 70, at least 80, at least 90, or at least 100). In some embodiments the ratio of the number of environmentally-responsive linkers to the number of catalytic nanoclusters is between 0.5 and 20, 1 and 20, 1 and 10, 1 and 30, 1 and 40, 1 and 50, 1 and 60, 5 and 10, 5 and 20, 10 and 20, or 1 and 100, inclusive.

### Scaffolds

The scaffold may serve as the core of the sensor (e.g., nanosensor). A purpose of the scaffold is to serve as a platform for the environmentally-responsive linker and enhance delivery of the sensor to tissue (e.g., disease tissue) in a subject. As such, the scaffold can be any material or size as long as it can enhance delivery and/or accumulation of the sensors to a tissue in a subject. Preferably, the scaffold material is non-immunogenic, i.e. does not provoke an immune response in the body of the subject to which it will be administered. Non-limiting examples of scaffolds, include, for instance, compounds that cause active targeting to tissue, cells or molecules (e.g., targeting of sensors to a tissue), microparticles, nanoparticles, aptamers, peptides (RGD, iRGD, LyP-1, CREKA, etc.), proteins, nucleic acids, polysaccharides, polymers, antibodies or antibody fragments (e.g., herceptin, cetuximab, panitumumab, etc.) and small molecules (e.g., erlotinib, gefitinib, sorafenib, etc.).

In some embodiments, the scaffold comprises a protein. For example, the scaffold may comprise a biotin-binding protein (e.g., avidin). Exemplary avidin proteins include, but are not limited to avidin, streptavidin, NeutrAvidin, and CaptAvidin.

In some embodiments, the scaffold has a diameter (e.g., hydrodynamic diameter) between 1 and10 nm, between 2.5 and 10 nm, between 3 and 10 nm, between 5 and 10 nm, between 6 and 10 nm, between 7 and 10 nm, between 8 and 10 nm, between 7 and 8 nm, between 9 and 10 nm, between 10 nm and 20 nm, or between 20 nm and 30 nm. In some instances, a scaffold has a diameter of 8 nm. In some embodiments, the scaffold has a diameter that is greater than 5 nm. In some embodiments, the scaffold is at least 6 nm, at least 7 nm, at least 8 nm, at least 9 nm, at least 10 nm, at least 20 nm, at least 30 nm, at least 40 nm, at least 50 nm, at least 60 nm, at least 70 nm, at least 80 nm, at least 90 nm, at least 100 nm, at least 200 nm, at least 300 nm, at least 400 nm, at least 500 nm, at least 600 nm, at least 700 nm, at least 800 nm, at least 900 nm, or at least 1,000 nm.

In some aspects, the disclosure relates to the discovery that delivery to a tissue in a subject is enhanced by sensors having certain polymer scaffolds (e.g., poly(ethylene glycol) (PEG) scaffolds). Polyethylene glycol (PEG), also known as poly(oxyethylene) glycol, is a condensation polymer of ethylene oxide and water having the general chemical formula HO(CH₂CH₂O)[n]H. Generally, a PEG polymer can range in size from about 2 subunits (e.g., ethylene oxide molecules) to about 50,000 subunits (e.g., ethylene oxide molecules. In some embodiments, a PEG polymer comprises between 2 and 10,000 subunits (e.g., ethylene oxide molecules).

A PEG polymer can be linear or multi-armed (e.g., dendrimeric, branched geometry, star geometry, etc.). In some embodiments, a scaffold comprises a linear PEG polymer. In some embodiments, a scaffold comprises a multi-arm PEG polymer. In some embodiments, a multi-arm PEG polymer comprises between 2 and 20 arms. Multi-arm and dendrimeric scaffolds are generally described, for example by Madaan et al. J Pharm Bioallied Sci. 2014 6(3): 139-150.

Additional polymers include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride and polystyrene.

Examples of non-biodegradable polymers include ethylene vinyl acetate, poly(meth) acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as algninate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. In some embodiments the polymers are polyesters, polyanhydrides, polystyrenes, polylactic acid, polyglycolic acid, and copolymers of lactic and glycoloic acid and blends thereof.

PVP is a non-ionogenic, hydrophilic polymer having a mean molecular weight ranging from approximately 10,000 to 700,000 and the chemical formula (C6H₉NO)[n]. PVP is also known as poly[1-(2-oxo-1-pyrrolidinyl)ethylen], Povidone™, Polyvidone™, RP 143™, Kollidon™, Peregal ST™, Periston™, Plasdone™, Plasmosan™, Protagent™ Subtosan™, and Vinisil™. PVP is non-toxic, highly hygroscopic and readily dissolves in water or organic solvents.

Polyvinyl alcohol (PVA) is a polymer prepared from polyvinyl acetates by replacement of the acetate groups with hydroxyl groups and has the formula (CH₂CHOH)[n]. Most polyvinyl alcohols are soluble in water.

PEG, PVA and PVP are commercially available from chemical suppliers such as the Sigma Chemical Company (St. Louis, Mo.).

In certain embodiments the polymer may comprise poly(lactic-co-glycolic acid) (PLGA).

In some embodiments, a scaffold (e.g., a polymer scaffold, such as a PEG scaffold) has a molecular weight equal to or greater than 40 kDa. In some embodiments, a scaffold is a particle (e.g., an iron oxide nanoparticle, IONP) that is between 10 nm and 50 nm in diameter (e.g. having an average particle size between 10 nm and 50 nm, inclusive). In some embodiments, a scaffold is a high molecular weight protein, for example an Fc domain of an antibody.

In some embodiments, one or more types of polymers are formed into nanoparticles (e.g., for use as a scaffold). In some embodiments, a scaffold is a branched polymer. In some embodiments, a scaffold is a nanoparticle comprised of polymers, which may further comprise at least one functional group for attaching a nanocatalyst (e.g., catalytic nanocluster). In some embodiments, a scaffold is a nanoparticle comprised of polymers and the scaffold encapsulates a nanocatalyst (e.g., catalytic nanocluster).

A preparation of particles, in some embodiments, includes particles having an average particle size of less than 1.0 μm in diameter or of greater than 1.0 μm in diameter but less than 1 mm. The preparation of particles may therefore, in some embodiments, have a diameter of at least 5, at least 10, at least 25, at least 50, or at least 75 microns, including sizes in ranges of 5-10 microns, 5-15 microns, 5-20 microns, 5-30 microns, 5-40 microns, or 5-50 microns. A composition of particles may have heterogeneous size distributions ranging from 10 nm to mm sizes. In some embodiments the diameter is about 5 nm to about 500 nm. In other embodiments, the diameter is about 100 nm to about 200 nm. In other embodiments, the diameter is about 10 nm to about 100 nm.

The scaffold may be composed of a variety of materials including iron, ceramic, metallic, natural polymer materials (including lipids, sugars, chitosan, hyaluronic acid, etc.), synthetic polymer materials (including poly-lactide-coglycolide, poly-glycerol sebacate, etc.), and non-polymer materials, or combinations thereof.

The scaffold may be composed in whole or in part of polymers or non-polymer materials. Non-polymer materials, for example, may be employed in the preparation of the particles. Exemplary materials include alumina, calcium carbonate, calcium sulfate, calcium phosphosilicate, sodium phosphate, calcium aluminate, calcium phosphate, hydroxyapatite, tricalcium phosphate, dicalcium phosphate, tricalcium phosphate, tetracalcium phosphate, amorphous calcium phosphate, octacalcium phosphate, and silicates. In certain embodiments the particles may comprise a calcium salt such as calcium carbonate, a zirconium salt such as zirconium dioxide, a zinc salt such as zinc oxide, a magnesium salt such as magnesium silicate, a silicon salt such as silicon dioxide or a titanium salt such as titanium oxide or titanium dioxide.

A number of biodegradable and non-biodegradable biocompatible polymers are known in the field of polymeric biomaterials, controlled drug release and tissue engineering (see, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d′Amore; and U.S. Pat. Nos. 4,806,621; 4,638,045 to Kohn; see also Langer, Acc. Chem. Res. 33:94, 2000; Langer, J. Control Release 62:7, 1999; and Uhrich et al., Chem. Rev. 99:3181, 1999; all of which are incorporated herein by reference).

The scaffold may be composed of inorganic materials. Inorganic materials include, for instance, magnetic materials, conductive materials, and semiconductor materials. In some embodiments, the scaffold is composed of an organic material (e.g., a biological material that enhances delivery of the sensor to a tissue of a subject).

In some embodiments, the scaffold is a porous particle. A porous particle can be a particle having one or more channels that extend from its outer surface into the core of the particle. In some embodiments, the channel may extend through the particle such that its ends are both located at the surface of the particle. These channels are typically formed during synthesis of the particle by inclusion followed by removal of a channel forming reagent in the particle.

The size of the pores may depend upon the size of the particle. In certain embodiments, the pores have a diameter of less than 15 microns, less than 10 microns, less than 7.5 microns, less than 5 microns, less than 2.5 microns, less than 1 micron, less than 0.5 microns, or less than 0.1 microns. The degree of porosity in porous particles may range from greater than 0 to less than 100% of the particle volume. The degree of porosity may be less than 1%, less than 5%, less than 10%, less than 15%, less than 20%, less than 25%, less than 30%, less than 35%, less than 40%, less than 45%, or less than 50%. The degree of porosity can be determined in a number of ways. For example, the degree of porosity can be determined based on the synthesis protocol of the scaffolds (e.g., based on the volume of the aqueous solution or other channel-forming reagent) or by microscopic inspection of the scaffolds post-synthesis.

The scaffold may be comprised of a plurality of particles which may be homogeneous for one or more parameters or characteristics. A plurality that is homogeneous for a given parameter, in some instances, means that particles within the plurality deviate from each other no more than about +/−10%, preferably no more than about +/−5%, and most preferably no more than about +/−1% of a given quantitative measure of the parameter. As an example, the particles may be homogeneously porous. This means that the degree of porosity within the particles of the plurality differs by not more than +/−10% of the average porosity. In other instances, a plurality that is homogeneous means that all the particles in the plurality were treated or processed in the same manner, including for example exposure to the same agent regardless of whether every particle ultimately has all the same properties. In still other embodiments, a plurality that is homogeneous means that at least 80%, preferably at least 90%, and more preferably at least 95% of particles are identical for a given parameter.

The plurality of particles may be heterogeneous for one or more parameters or characteristics. A plurality that is heterogeneous for a given parameter, in some instances, means that particles within the plurality deviate from the average by more than about +/−10%, including more than about +/−20%. Heterogeneous particles may differ with respect to a number of parameters including their size or diameter, their shape, their composition, their surface charge, their degradation profile, whether and what type of agent is comprised by the particle, the location of such agent (e.g., on the surface or internally), the number of agents comprised by the particle, etc. The disclosure contemplates separate synthesis of various types of particles which are then combined in any one of a number of pre-determined ratios prior to contact with the sample. As an example, in one embodiment, the particles may be homogeneous with respect to shape (e.g., at least 95% are spherical in shape) but may be heterogeneous with respect to size, degradation profile and/or agent comprised therein.

Scaffold size, shape and release kinetics can also be controlled by adjusting the scaffold formation conditions. For example, scaffold formation conditions can be optimized to produce smaller or larger scaffolds, or the overall incubation time or incubation temperature can be increased.

The scaffold may be formulated, for instance, into liposomes, virosomes, cationic lipids or other lipid based structures. The term “cationic lipid” refers to lipids which carry a net positive charge at physiological pH. Such lipids include, but are not limited to, DODAC, DOTMA, DDAB, DOTAP, DC-Chol and DMRIE. Additionally, a number of commercial preparations of cationic lipids are available. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic lipids comprising DOGS in ethanol from Promega Corp., Madison, Wis., USA). A variety of methods are available for preparing liposomes e.g., U.S. Pat. Nos. 4,186,183, 4,217,344, 4,235,871, 4,261,975, 4,485,054, 4,501,728, 4,774,085, 4,837,028, 4,946,787; and PCT Publication No. WO 91/17424. The particles may also be composed in whole or in part of GRAS components. i.e., ingredients are those that are Generally Regarded As Safe (GRAS) by the US FDA. GRAS components useful as particle material include non-degradable food based particles such as cellulose. In some embodiments, a scaffold is a liposome comprising phosphatidylcholine (POPC). For example, the liposome may comprise a lipid bilayer that comprises POPC. As a non-limiting example, a liposome comprising POPC may be ruptured in the presence of the enzyme phospholipase A2 (PLA₂). In some embodiments, a liposome comprising POPC that encapsulates a nanocatalyst may release the nanocatalyst in the presence of PLA₂.

In some embodiments, a scaffold is a liposome comprising brain sphingomyelin (BSM) and cholesterol (CH). For example, the liposome may comprise a lipid bilayer that comprises BSM and CH. The ratio of BSM to CH may be at least 1:1, at least 1:2, at least 2:1, at least 3:1, at least 1:3, at least 1:4, at least 4:1, at least 5:1, at least 1:5, at least 2:3, at least 3:2, at least 3:4, at least 4:3, at least 5:4, at least 4:5, at least 10:1, or at least 1:10. As a non-limiting example, a liposome comprising BSM and CH may be ruptured in the presence of the enzyme sphingomyelinase (SMase) or a toxin. In some instances, the toxin is a bacterial toxin that is capable of forming a pore (e.g., alpha hemolysin). In some embodiments, a liposome comprising BSM and CH and encapsulates a nanocatalyst releases the nanocatalyst in the presence of sphingomyelinase (SMase) and/or a toxin (e.g., a pore-forming toxin). In some embodiments, a liposome comprising BSM and CH and encapsulates a nanocatalyst releases the nanocatalyst in the presence of the enzyme sphingomyelinase (SMase) and/or toxins (including alpha-hemolysin) from Staphylococcus aureus. In some embodiments, the sphingomyelinase (SMase) and/or toxins are present in Staphylococcus aureus bacterial supernatants.

The scaffold can serve several functions. As discussed above, it may be useful for targeting the product to a specific region, such as tissue. In that instance, it could include a targeting agent such as a glycoprotein, an antibody, or a binding protein.

Further, the size of the scaffold may be adjusted based on the particular use of the in vivo sensor. For instance, the scaffold may be designed to have a size greater than 5 nm. Particles, for instance, of greater than 5 nm are not capable of entering the urine, but rather, are cleared through the reticuloendothelial system (RES; liver, spleen, and lymph nodes). By being excluded from the removal through the kidneys any uncleaved sensor will not be detected in the urine during the analysis step. Additionally, larger particles can be useful for maintaining the particle in the blood or in a tumor site where large particles are more easily shuttled through the vasculature. In some embodiments the scaffold is 500 microns-5nm, 250 microns-5 nm, 100 microns-5nm, 10 microns-5 nm, 1 micron-5 nm, 100 nm-5 nm, 100 nm-10 nm, 50nm-10nm or any integer size range therebetween. In other instances the scaffold is smaller than 5 nm in diameter. In such instance, the sensor will be cleared into the urine. However, the presence of free nanocatalyst (as opposed to a nanocatalyst still attached to an uncleaved environmentally-sensitive linker) can be detected for instance using mass spectrometry. In some embodiments the scaffold is 1-5 nm, 2-5 nm, 3-5 nm, or 4-5 nm in diameter.

Optionally the scaffold may include a biological agent. In one embodiment, a biological agent could be incorporated in the scaffold or it may make up the scaffold. Thus, the compositions of the invention can achieve two purposes at the same time, the diagnostic methods and delivery of a therapeutic agent. In some embodiments, the biological agent may be an enzyme inhibitor. In that instance the biological agent can inhibit proteolytic activity at a local site and the nanocatalyst can be used to test the activity of that particular therapeutic at the site of action.

Nanocatalysts

Nanocatalysts, as used herein, are nanoscale particles comprising catalytically active materials (e.g., comprising a surface of catalytically active materials, comprising a core of catalytically active materials, or any combination thereof). For example, nanocatalysts include particles smaller than 100 nm in at least one dimension, particles smaller than 1 nm in at least one dimension, particles 1 nm in at least one dimension, particles greater than 1 nm in at least one dimension, particles between 1 nm and 300 nm in at least one dimension, dimension, and particles great than 300 nm, but less than 1,000 nm in at least one dimension. In some examples, nanocatalysts are porous compounds having pore diameters not bigger than 100 nm, having pore diameters not bigger than 300 nm, having pore diameters not bigger than 1 nm, having pore diameters not bigger than 1,000 nm. In some embodiments, a nanocatalyst comprises a catalytically active shell or coating. In some embodiments, a nanocatalyst is composed entirely of a catalytically active material.

A nanocatalyst may be less than 10 nm (e.g., less than 9 nm, less than 8 nm, less than 7 nm, less than 6 nm, less than 5 nm, less than 4.5 nm, less than 4 nm, less than 3.5 nm, less than 3 nm, less 2.5 nm, less than 2 nm, less than 1.5 nm, or less than 1 nm) in diameter. In some preferred embodiments, the nanocatalyst is less than 5 nm in diameter. In other embodiments the nanocatalyst is 1-5 nm, 1-4 nm, 1-3 nm, 1-2 nm, 2-5 nm, 2-4 nm, 2-3 nm, 3-4 nm, 3-5 nm or 4-5 nm in diameter. In some embodiments, a nanocatalyst is between 1 and 300 nm in diameter. In some embodiments, a nanocatalyst is bigger than 300 nm in diameter.

Exemplary nanocatalysts include catalytic nanoclusters. For example, catalytic nanoclusters may be made of transition metals, including gold, iron, silver, palladium, iridium and platinum. In some embodiments, a transition metal is a noble metal (e.g., gold, silver, platinum, etc.). As used herein, nanoclusters (e.g., transition metal nanoclusters) are colloids that comprise at least two atoms (e.g., at least 3, at least 4, at least 5, at least 6, at least 10, at least 20, at least 30, at least 40, at least 50, at least 60, at least 70, at least 80, at least 90, at least 100, at least 200, at least 300, at least 400, at least 500, at least 600, at least 700, at least 800 at least 900, or at least 1,000 atoms). A nanoclusters may be less than 10 nm (e.g., less than 9 nm, less than 8 nm, less than 7 nm, less than 6 nm, less than 5 nm, less than 4.5 nm, less than 4 nm, less than 3.5 nm, less than 3 nm, less 2.5 nm, less than 2 nm, less than 1.5 nm, or less than 1 nm) in diameter.

Exemplary nanocatalysts include iron oxide nanoparticles and iridium nanoparticles. A nanocatalyst may be less than 10 nm (e.g., less than 9 nm, less than 8 nm, less than 7 nm, less than 6 nm, less than 5 nm, less than 4.5 nm, less than 4 nm, less than 3.5 nm, less than 3 nm, less 2.5 nm, less than 2 nm, less than 1.5 nm, or less than 1 nm) in diameter.

In some embodiments, a nanocatalyst (e.g., catalytic nanocluster) is linked to a capping agent. Non-limiting examples of capping agents include organic ligands, polymers, and surfactants. In some instances, a nanocatalyst comprises a zwitterionic peptide capping layer (e.g., an environmentally-responsive linker may act as a capping agent).

Capping agents may be used to control the size or shape of a nanocatalyst. In some instances, a capping agent helps retain the catalytic activity of a nanocatalyst. For example, a nanocatalyst with a capping agent may have a catalytic activity that is at least 10% (at least 20%, at least 30%, at least 40%, at least 50%, at least 60%, at least 70%, at least 80%, at least 90%, or 100%) that of a nanocatalyst without a capping agent. Without being bound by a particular theory, a capping agent (e.g., a layer of capping agents) may allow for a nanocatalyst to retain catalytic activity under physiological conditions (e.g., in protein-rich environments). In some embodiments, a protein-rich environment comprises at least 0.1 mg/dL, at least 0.2 mg/dL, at least 0.3 mg/dL, at least 0.4 mg/dL, at least 0.5 mg/dL, at least 0.6 mg/dL, at least 0.7 mg/dL, at least 0.8 mg/dL, at least 0.9 mg/dL, at least 1 mg/dL, at least 10 mg/dL, at least 50 mg/dL, at least 100 mg/dL, at least 500 mg/dL, at least 1,000 mg/dL, at least 2,000 mg/dL, at least 3,000 mg/dL, at least 4,000 mg/dL, at least 5,000 mg/dL, at least 6,000 mg/dL, or at least 7,000 mg/dL total protein. In some embodiments, a protein-rich environment comprises up to 7 wt % protein (i.e., up to 7,000 mg/dL). In some embodiments, a protein-rich environment comprises up to 1 mg/dL, up to 10 mg/dL, up to 50 mg/dL, up to 100 mg/dL, up to 500 mg/dL, up to 1,000 mg/dL, up to 2,000 mg/dL, up to 3,000 mg/dL, up to 4,000 mg/dL, up to 5,000 mg/dL, up to 6,000 mg/dL, or up to 7,000 mg/dL total protein. Without being bound by a particular theory, the nanocatalysts described herein may retain catalytic activity after serum exposure due to surface capping layer and size, which may prevent protein fouling and/or binding to the nanocatalyst's surface and prevent protein from knocking out catalytic surface area on the nanoparticle.

The surface area to volume ratio of a nanocatalyst (e.g., catalytic nanocluster) may be modulated to alter its catalytic activity. For example, a nanocatalyst may have a high surface area to volume ratio (e.g., a ratio that is greater than 1, greater than 1.1, greater than 1.2, greater than 1.3, greater than 1.4, greater than 1.5, greater than 1.6, greater than 1.7, greater than 1.8, greater than 1.9, greater than 2, greater than 2.5, greater than 3, greater than 3.5, greater than 4, greater than 4.5, greater than 5, greater than 5.5, or greater than 6). In some embodiments a nanocatalyst has a surface area between 1.1 and 6 (e.g., between 1.2 and 6, between 1.3 and 6, between 1.4 and 6, between 1.5 and 6, between 2 and 6, between 3 and 6, between 4 and 6, between 5 and 6, between 1 and 2, between 2 and 3, between 3 and 4, or between 4 and 5).

A nanocatalyst (e.g., catalytic nanocluster) may be detected using any suitable method. Detection of a nanocatalyst may include detection of luminescence, fluorescence or a colorimetric assay. For example, the catalytic activity of a nanocatalyst may be detected (e.g., quantified). A nanocatalyst may be capable of promoting oxidation (e.g., capable of disproportionating H₂O₂). A non-limiting example of an oxidation assay includes assays that use a peroxidase substrate. Exemplary peroxidase substrates include chromogenic substrates (e.g., 3,3′,5,5′-Tetramethylbenzidine (TMB), 4-chloro-1-naphthol (4CN), 2,2′-azino-di-[3-ethylbenzthiazoline-6-sulfonic acid] (ABTS), AEC, OPD, or 3,3′-diaminobenzidine (DAB)). Oxidized substrates may then be measured and quantified using colorimetric assays (e.g., by determining absorbance of a sample at a given wavelength), luminescence assays, fluorescence assays, and enzyme-linked immunosorbent assays (ELISAs).

As a non-limiting example, the solubility of a product formed from a substrate following an oxidation reaction could allow for different readouts. For example, for an oxidation assay that results in a soluble product, the soluble product could be detected using a plate reader. For an insoluble product, the product could be deposited on a membrane, which could be detected, using for example, a western blot. The amount of the product could be quantified and correlated to the activity of the nanocatalysts of interest.

In some instances, a substrate is a chemiluminescent substrate. In some instances, a substrate is suitable for detection of HRP (e.g., in an ELISA). For example, the substrate may be chromogenic, chemiluminescent, or fluorogenic.

In some embodiments, a nanocatalyst (e.g., catalytic nanocluster) does not exhibit surface plasmon resonance (e.g., at 520 nm). In some embodiments, a nanocatalyst (e.g., catalytic nanocluster) exhibits molecular-like absorption. In some embodiments, a nanocatalyst (e.g., catalytic nanocluster) exhibits fluorescence properties (e.g., with an emission peak at 600 nm).

In some embodiments, the catalytic activity of a nanocatalyst is measured by determining the catalytic constant (Kcat) in s⁻¹ units. Kcat may be determined by dividing the maximal reaction velocity (Vmax) and the catalyst concentration ([E]). In some embodiments, a nanocatalyst has a high Kcat value (e.g., Kcat value that is greater than 10⁴ s⁻¹). In some embodiments, a nanocatalyst has a Kcat that is at least 1×10¹ s⁻¹, at least 1×10² s⁻¹, at least 1×10³ s⁻¹, at leastlx10⁴ s⁻¹, at least 1×10⁵ s⁻¹, at least 1×10⁶ s⁻¹, at least 1×10⁷ s⁻¹, at least 1×10⁸ s⁻¹, at least 1×10⁹ s⁻¹, at least 1×10¹⁰ s⁻¹, at least 1×10¹¹ s⁻¹, at least 1×10¹² s⁻¹, at least 1×10¹³ s⁻¹, at least 1×10¹⁴ s⁻¹, at least 1×10¹⁵ s⁻¹, at least 1×10¹⁶ s⁻¹, at least 1×10¹⁷ s⁻¹, at least 1×10¹⁸ s⁻¹, at least 1×10¹⁹ s⁻¹, at least 1×10²⁰ s⁻¹, at least 1×10⁵⁰ s⁻¹, or at least 1×10¹⁰⁰ s⁻¹. In some embodiments, a nanocatalyst has a Kcat that is less than 1×10¹⁰ s⁻¹, less than 1×10⁹ s⁻¹, less than 1×10⁸s⁻¹, less than 1×10⁷ s⁻¹, less than 1×10⁶ s⁻¹, less than 1×10⁵ s⁻¹, less than 1×10⁴ s⁻¹, less than 5×10³ s⁻¹, less than 10×10² s⁻¹, or less than 1×10 s⁻¹.

### Linkers

As used herein “linked” or “linkage” means two entities are bound to one another by any physicochemical means. Any linkage known to those of ordinary skill in the art, covalent or non-covalent, is embraced. Thus, in some embodiments the scaffold has a linker (e.g., environmentally-responsive linker) attached to an external surface, which can be used to link the nanocatalyst.

The in vivo sensors of the present disclosure comprise an environmentally-responsive linker that is located between the scaffold and the nanocatalyst. An environmentally-responsive linker, as used herein, is the portion of the sensor that changes in structure in response to an environmental trigger in the subject, causing the release of a nanocatalyst. Thus, an environmentally-responsive linker has two forms. The original form of the linker is attached to the scaffold and the nanocatalyst. When exposed to an environmental trigger the linker is modified in some way. For instance, it may be cleaved by an enzyme such that the nanocatalyst is released. Alternatively it may undergo a conformational change which leads to release of the nanocatalyst.

In some embodiments, an environmentally responsive linker is directly linking the nanocluster to the scaffold. In some embodiments, a scaffold comprises an environmentally responsive linker that encapsulates a nanocatalyst (e.g., catalytic nanocluster).

Certain environmental triggers present in a disease microenvironments have been associated with disease. For example, environmental triggers include enzymes, light, pH, and temperature. An enzyme, as used herein refers to any of numerous proteins produced in living cells that accelerate or catalyze the metabolic processes of an organism. Enzymes act on substrates. The substrate binds to the enzyme at a location called the active site just before the reaction catalyzed by the enzyme takes place. Enzymes include but are not limited to proteases, glycosidases, lipases, heparinases, and phosphatases. In some instances, an environmental linker comprises a photolabile group, which may change conformation in response to light (e.g., to a particular wavelength of light).

Dysregulated protease activities are implicated in a wide range of human diseases; including cancer, pulmonary embolism, inflammation, and infectious diseases, such as, bacterial infections, viral infections (e.g., HIV) and malaria. A sensor of the present disclosure may be used to detect an endogenous and/or an exogenous protease. An endogenous protease is a protease that is naturally produced by a subject (e.g., subject with a particular disease or a host with an infection). An exogenous protease is a protease that is not naturally produced by a subject and may be produced by a pathogen (e.g., a bacteria, a fungi, protozoa, or a virus). In some embodiments, a protease is only expressed by a subject (e.g., a human) and not by pathogen. In some embodiments, a protease is pathogen-specific and is only produced by a pathogen not by the pathogen's host.

Table 1 provides a non-limiting list of enzymes associated with (either increased or decreased with respect to normal) disease and in some instances, the specific substrate. Table 2 provides a non-limiting list of substrates associated with disease or other conditions. Numerous other enzyme/substrate combinations associated with specific diseases or conditions are known to the skilled artisan and are useful according to the invention.

TABLE 1
Non-limiting examples of disease-associated enzymes and substrates.

| Disease | Enzyme | Substrate |
|---|---|---|
| Cancer | MMP | collagens, gelatin, |
| | | various ECM |
| | | proteins |
| Cancer | MMP-2 | type IV collagen and |
| | | gelatin |
| Cancer | MMP-9 | type IV and V |
| | | collagens and gelatin |
| Cancer | Kallikreins | kininogens, |
| | | plasminogen |
| Cancer | Cathepsins | broad spectrum of |
| | | substrates |
| Cancer | plasminogen activator, tPA | Plasminogen |
| Cancer | Urokinase-type plasminogen | Plasminogen |
| | activator, uPA | |
| Cancer | ADAM (A Diseintegrin And | various extracellular |
| | Metalloprotease, also MDC, | domains of |
| | Adamalysin) | transmembrane |
| | | proteins |
| Pancreatic carcinoma | MMP-7 | various, e.g. |
| | | collagen 18, FasL, |
| | | HLE, DCN, IGFBP- |
| | | 3, MAG, |
| | | plasminogen, other |
| | | MMPs |
| Pancreatic Cancer | ADAM9, ADAM15 | various extracellular |
| | | domains of |
| | | transmembrane |
| | | proteins |
| Prostate adenocarcinoma | Matriptase, a type II | unspecific, cleaves |
| | transmembrane serine protease | after Lys or Arg |
| | | residues |
| Prostate cancer | Kallikrein 3 | kininogens, |
| | | plasminogen |
| Prostate cancer | ADAM15 | various extracellular |
| | | domains of |
| | | transmembrane |
| | | proteins |
| Ovarian carcinoma | Kallikrein 6 | kininogens, |
| | | plasminogen |
| Epithelial-derived tumors | Matriptase, a type II | unspecific, cleaves |
| (breast, prostate, ovarian, colon, | transmembrane serine protease | after Lys or Arg |
| oral) | | residues |
| Ovarian Cancer | MMP-2, MMP-9, kallikrein-10 | type IV and V |
| | (hk-10) | collagens and |
| | | gelatin, kininogens, |
| | | plasminogen |
| Breast, gastric, prostate cancer | cathepsins B, L and D | broad spectrum of |
| | | substrates |
| Endometrial cancer | cathepsin B | unspecific cleavage |
| | | of a broad spectrum |
| | | of substrates without |
| | | clear sequence |
| | | specificity |
| esophageal adenocarcinoma | cathepsin B | unspecific cleavage |
| | | of a broad spectrum |
| | | of substrates without |
| | | clear sequence |
| | | specificity |
| Invasive cancers, metastases | type II integral serine proteases | |
| | (dipeptidyl peptidase IV | |
| | (DPP4/CD26), seprase/fibroblast | |
| | activation protein alpha | |
| | (FAPalpha) and related type II | |
| | transmembrane prolyl serine | |
| | peptidases)) | |
| Invasive cancers, metastases | Seprase | various ECM |
| | | proteins |
| Viral Infections | | |
| All Retroviruses | viral protease | precursor GagPol |
| | | fusion |
| HIV | HIV protease (HIV PR, an | precursor Gag and |
| | aspartic protease) | GagPol proteins |
| Hepatitis C | NS3 serine protease | viral precursor |
| | | polyprotein |
| Dengue | Dengue protease | autocleavage |
| | | (NS2B/NS3), |
| | | NS3/NS4A and |
| | | NS4B/NS5 cleavage |
| West Nile | NS2B/NS3pro | viral precursor |
| | | polyprotein |
| Bacterial Infections | | |
| Legionella spp. | zinc metalloprotease | Me-Arg-Pro-Tyr |
| Meninogencephalitis | histolytic cysteine protease | |
| Streptococcus pyogenes (Group | streptococcal pyrogenic exotoxin | extracellular matrix, |
| A Streptococcus) | B (SpeB) | immunoglobulins, |
| | | complement |
| | | components |
| Clostridium difficile | Cwp84 | fibronectin, laminin, |
| | | vitronectin and other |
| | | ECM proteins |
| Pseudomonas aeruginosa | lasA | Leu-Gly-Gly-Gly- |
| | | Ala |
| Pseudomonas aeruginosa | Large ExoProtease A | Cleavage of peptide |
| | | ligands on PAR1, |
| | | PAR2, PAR4 |
| | | (Protease-activated |
| | | receptor). See, e.g., |
| | | Kida et al, Cell |
| | | Microbiol. 2008 |
| | | July; 10(7):1491-504. |
| Pseudomonas aeruginosa | protease IV | complement factors, |
| | | fibrinogen, |
| | | plasminogen (See, |
| | | e.g., Engel et al., J |
| | | Biol Chem. 1998 Jul. |
| | | 3; 273(27):16792-7). |
| Pseudomonas aeruginosa | alkaline protease | Complement factor |
| | | C2 (See, e.g., |
| | | Laarman et al., J |
| | | Immunol. 2012 Jan. |
| | | 1; 188(1):386-93). |
| Additional Diseases | | |
| Alzheimer's disease | BACE-1,2 (Alzheimer secretase) | β-amyloid precursor |
| | | protein |
| Stroke and recovery | MMP, tPA | |
| cardiovascular disease | Angiotensin Converting Enzyme | angiotensin I, |
| | (ACE) | bradykinin |
| Atherosclerosis | cathepsin K, L, S | broad spectrum of |
| | | substrates |
| Arthritis | MMP-1 | triple-helical fibrillar |
| | | collagens |
| rheumatoid arthritis | thrombin | Osteopontin |
| Malaria | SUB1 | KITAQDDEES |
| osteoarthritis | thrombin | Osteopontin |
| osteoporosis/osteoarthritis | cathepsin K, S | broad spectrum of |
| | | substrates |
| Arthritis, inflammatory joint | Aggrecanase (ADAMTS4, | aggrecans |
| disease | ADAMTS11) | (proteoglycans) |
| thrombosis | factor Xa (thrombokinase) | Prothrombin |
| thrombosis | ADAMTS13 | von Willebrand |
| | | factor (vWF) |
| thrombosis | plasminogen activator, tPA | Plasminogen |
| Stress-induced Renal pressure | Prostasin | epithelial Na |
| natriuresis | | channel subunits |

TABLE 2
Non-limiting examples of substrates associated with disease and other
conditions.

| DISEASE | TARGET SUBSTRATE | ENZYME |
|---|---|---|
| Inflammation | Interleukin 1 beta | MMP-2, MMP-3, MMP-9, |
| | | Trypsin, chymotrypsin, pepsin, |
| | | Lys-C, Glu-C, Asp-N, Arg-C |
| Pituitary gland | IGFBP-3 | MMP-1, MMP-3, MMP-9, |
| dysfunction, abnormal | | Trypsin, chymotrypsin, pepsin, |
| bone density, growth | | Lys-C, Glu-C, Asp-N, Arg-C |
| disorders | | |
| Cancer | TGF-beta | MMP-9, Trypsin, chymotrypsin, |
| | | pepsin, Lys-C, Glu-C, Asp-N, |
| | | Arg-C |
| Cancer, autoimmune | TNF | MMP-7, Trypsin, chymotrypsin, |
| disease | | pepsin, Lys-C, Glu-C, Asp-N, |
| | | Arg-C |
| Cancer, autoimmune | FASL | MMP-7, Trypsin, chymotrypsin, |
| disease | | pepsin, Lys-C, Glu-C, Asp-N, |
| | | Arg-C |
| Wound healing, cardiac | HB-EGF | MMP-3, Trypsin, chymotrypsin, |
| disease | | pepsin, Lys-C, Glu-C, Asp-N, |
| | | Arg-C |
| Pfeiffer syndrome | FGFR1 | MMP-2, Trypsin, chymotrypsin, |
| | | pepsin, Lys-C, Glu-C, Asp-N, |
| | | Arg-C |
| Cancer | Decorin | MMP-2, MMP-3, MMP-7, |
| | | Trypsin, chymotrypsin, pepsin, |
| | | Lys-C, Glu-C, Asp-N, Arg-C |
| Cancer | Tumor associated | Endoglycosidases |
| | carbohydrate antigens | |
| Cancer | Sialyl Lewis^{a} | O-glycanase |
| Cancer | Sialyl Lewis^{X} | O-glycanase |
| Cancer/Rheumatoid | VEGF | Trypsin, chymotrypsin, pepsin, |
| Arthritis, pulmonary | | Lys-C, Glu-C, Asp-N, Arg-C |
| hypertension | | |
| Cancer | EGF | Trypsin, chymotrypsin, pepsin, |
| | | Lys-C, Glu-C, Asp-N, Arg-C |
| Cancer | IL2 | Trypsin, chymotrypsin, pepsin, |
| | | Lys-C, Glu-C, Asp-N, Arg-C |
| Cancer | IL6 | Trypsin, chymotrypsin, pepsin, |
| inflammation/angiogenesis | | Lys-C, Glu-C, Asp-N, Arg-C |
| Cancer | IFN-γ | Trypsin, chymotrypsin, pepsin, |
| | | Lys-C, Glu-C, Asp-N, Arg-C |
| Cancer | TNF-α | Trypsin, chymotrypsin, pepsin, |
| inflammation/angiogenesis, | | Lys-C, Glu-C, Asp-N, Arg-C |
| Rheumatoid Arthritis | | |
| Cancer, Pulmonary | TGF-β | Trypsin, chymotrypsin, pepsin, |
| fibrosis, Asthma | | Lys-C, Glu-C, Asp-N, Arg-C |
| Cancer, Pulmonary | PDGF | Trypsin, chymotrypsin, pepsin, |
| hypertension | | Lys-C, Glu-C, Asp-N, Arg-C |
| Cancer, pulmonary | Fibroblast growth factor | Trypsin, chymotrypsin, pepsin, |
| cystadenoma | (FGF) | Lys-C, Glu-C, Asp-N, Arg-C |
| Cancer | Brain-derived | Trypsin, chymotrypsin, pepsin, |
| | neurotrophic factor | Lys-C, Glu-C, Asp-N, Arg-C |
| | (BDNF) | |
| Cancer | Interferon regulatory | Trypsin, chymotrypsin, pepsin, |
| | factors (IRF-1, IRF-2) | Lys-C, Glu-C, Asp-N, Arg-C |
| Inhibitor of tumor | MIF | Trypsin, chymotrypsin, pepsin, |
| suppressors | | Lys-C, Glu-C, Asp-N, Arg-C |
| Lymphomas/carcinomas, | GM-CSF | Trypsin, chymotrypsin, pepsin, |
| alveolar proteinosis | | Lys-C, Glu-C, Asp-N, Arg-C |
| Cancer invasion | M-CSF | Trypsin, chymotrypsin, pepsin, |
| | | Lys-C, Glu-C, Asp-N, Arg-C |
| Chemical carcinogenesis, | IL-12 | Trypsin, chymotrypsin, pepsin, |
| multiple sclerosis, | | Lys-C, Glu-C, Asp-N, Arg-C |
| rheumatoid arthritis, | | |
| Crohn's disease | | |
| Natural Killer T cell | IL-15 | Trypsin, chymotrypsin, pepsin, |
| leukemias, inflammatory | | Lys-C, Glu-C, Asp-N, Arg-C |
| bowel disease, rheumatoid | | |
| arthritis | | |
| Cirrhosis | Tissue inhibitor of MMPs | Trypsin, chymotrypsin, pepsin, |
| | (TIMPs) | Lys-C, Glu-C, Asp-N, Arg-C |
| Cirrhosis | Collagen I, III | MMP-1, MMP-8, Trypsin, |
| | | chymotrypsin, pepsin, Lys-C, |
| | | Glu-C, Asp-N, Arg-C |
| Cirrhosis | Collagen IV, V | MMP-2, Trypsin, chymotrypsin, |
| | | pepsin, Lys-C, Glu-C, Asp-N, |
| | | Arg-C |

Non-limiting examples of enzyme cleavable linkers may also be found in WO2010/101628, entitled METHODS AND PRODUCTS FOR IN VIVO ENZYME PROFILING, which was filed on Mar. 2, 2010.

A disease microenvironment may have a pH that deviates from a physiological pH. Physiological pH may vary depending on the subject. For example, in humans, the physiological pH is generally between 7.3 and 7.4 (e.g., 7.3, 7.35, or 7.4). A disease microenvironment may have a pH that is higher (e.g., more basic) or lower (e.g., more acidic) than a physiological pH. As an example, acidosis is characterized by an acidic pH (e.g., pH of lower than 7.4, a pH of lower than 7.35, or a pH of lower than 7.3) and is caused by metabolic and respiratory disorders. Non-limiting examples of diseases associated with acidosis include cancer, diabetes, kidney failure, chronic obstructive pulmonary disease, pneumonia, asthma and heart failure. In some embodiments, an acidic pH induces cleavage of an environmentally-responsive linker and releases a nanocatalyst from an in vivo sensor. Additional pH-responsive linkers include hydrazones and cis-Aconityl linkers. For example, hydrazones or cis-Aconityl linkers can be used to attach a nanocatalyst (e.g., catalytic nanocluster) to the scaffold and the linker undergoes hydrolysis in an acidic environment.

Another non-limiting example of an environmentally-responsive linker is a temperature-sensitive linker that changes structure at a particular temperature (e.g., a temperature above or below 37 degrees Celsius). In some instances, a temperature above 37 degrees Celsius (e.g., as indicative of a fever associated with influenza) induces cleavage of an environmentally-responsive linker and releases a nanocatalyst from an in vivo sensor. In some embodiments, a temperature-sensitive linker is linked (e.g., tethered) to a scaffold.

In some embodiments, a temperature-sensitive linker undergoes a conformational change in response to a particular temperature. As a non-limiting example, a scaffold may be composed of one or more temperature-sensitive linkers encapsulating a nanocatalyst and in response to a particular temperature, the scaffold may become leaky and release the nanocatalyst. In one embodiment, a nanocatalyst is encapsulated (e.g., in a polymerosome, liposome, particle) by a temperature-sensitive linker, which is composed of NIPAM polymer. In some embodiments, the NIPAM polymer becomes leaky at one or more temperatures and releases an encapsulated nanocatalyst.

In some embodiments, a scaffold comprises one or more environmentally-sensitive linkers (e.g., an environmentally-sensitive linker that is responsive to pH, light, temperature, enzymes, light, or a combination thereof) and the scaffold encapsulates a nanocatalyst. In some instances, the scaffold encapsulating a nanocatalyst becomes degraded or leaky in response to a particular pH, temperature, presence of an enzyme, or light (e.g., a particular wavelength of light) and releases the nanocatalyst. In some embodiments, a scaffold encapsulating a nanocatalyst is a liposome, a polymersome, or a PLGA nanoparticle.

An environmentally-responsive linker (e.g., enzyme substrate, pH-sensitive linker, or a temperature-sensitive linker) may be attached directly to the scaffold. For instance it may be coated directly on the surface of the scaffold using known techniques. Alternatively if the scaffold is a protein material it may be directly connected through a peptide bond. Additionally, the environmentally-responsive linker may be connected to the scaffold through the use of another linker. Thus, in some embodiments the scaffold may be attached directly to the environmentally-responsive linker or indirectly through another linker. The other linker may simply be a spacer (or in other works be a linker that is not responsive to an environmental trigger). Another molecule can also be attached to a linker. In some embodiments, two molecules are linked using a transpeptidase, for example Sortase A.

Examples of linking molecules include but are not limited to poly(ethylene glycol), peptide linkers, N-(2-Hydroxypropyl) methacrylamide linkers, elastin-like polymer linkers, and other polymeric linkages. Generally, a linking molecule is a polymer and may comprise between about 2 and 200 (e.g., any integer between 2 and 200, inclusive) molecules. In some embodiments, a linking molecule comprises one or more poly(ethylene glycol) (PEG) molecules. In some embodiments, a linking molecule comprises between 2 and 200 (e.g., any integer between 2 and 200, inclusive) PEG molecules. In some embodiments, a linking molecule comprises between 2 and 20 PEG molecules. In some embodiments, a linking molecule comprises between 5 and 15 PEG molecules. In some embodiments, a linking molecule comprises between 5 and 25 PEG molecules. In some embodiments, a linking molecule comprises between 10 and 40 PEG molecules. In some embodiments, a linking molecule comprises between 25 and 50 PEG molecules. In some embodiments, a linking molecule comprises between 100 and 200 PEG molecules.

In other embodiments, the second linker may be a second environmentally-responsive linker. The use of multiple environmentally-responsive linkers allows for a more complex interrogation of an environment. For instance, a first linker may be sensitive to a first environmental condition or trigger and upon exposure to an appropriate trigger undergoes a conformational change which exposes the second environmentally-responsive linker. When a second trigger is also present then the second environmentally-responsive linker may be engaged in order to release the nanocatalyst for detection. Only the presence of the two triggers in one environment would enable the detection of the nanocatalyst.

The sensitivity and specificity of an in vivo sensor may be improved by modulating presentation of the environmentally-responsive linker to its cognate environmental trigger, for example by varying the distance between the scaffold and the environmentally responsive linker of the in vivo sensor. For example, in some embodiments, a polymer comprising one or more linking molecules is used to adjust the distance between a scaffold and an environmentally-responsive linker, thereby improving presentation of the environmentally responsive linker to its cognate environmental trigger.

In some embodiments, the distance between a scaffold and an environmentally-responsive linker (e.g., enzyme substrate, pH-sensitive linker, or temperature-sensitive linker) ranges from about 1.5 angstroms to about 1000 angstroms. In some embodiments, the distance between a scaffold and an environmentally-responsive linker ranges from about 10 angstroms to about 500 angstroms (e.g., any integer between 10 and 500). In some embodiments, the distance between a scaffold and a substrate ranges from about 50 angstroms to about 800 angstroms (e.g., any integer between 50 and 800). In some embodiments, the distance between a scaffold and a substrate ranges from about 600 angstroms to about 1000 angstroms (e.g., any integer between 600 and 1000). In some embodiments, the distance between a scaffold and a substrate is greater than 1000 angstroms.

In some embodiments, a sensor described herein comprises a spacer, which may be useful in reducing steric hindrance of an environmental trigger from accessing an environmentally-responsive linker. In some embodiments, a spacer comprises at least 1, 2, 3, 4, 5, 6, 7, 8, 9, 10, 11, 12, 13, 14, 15, 16, 17, 18, 19, 20, 30, 40, 50, 60, 70, 80, or 90 amino acids (e.g., glycine). In some embodiments, a spacer is a polyethelyne glycol (PEG) spacer (e.g., a PEG spacer that is at least 100 Da, at least 200 Da, at least 300 Da, at least 400 Da, at least 500 Da, at least 600 Da, at least 700 Da, at least 800 Da, at least 900 Da, at least 1,000 Da, at least 2,000 Da, at least 3,000 Da, at least 4,000 Da, at least 5,000 Da, at least 6,000 Da, at least 7,000 Da, at least 8,000 Da, at least 9,0000 Da or at least 10,000 Da). In some embodiments, a PEG spacer is between 200 Da and 10,000 Da. In some embodiments, a spacer sequence is located between a scaffold and an environmentally-sensitive linker. In some embodiments, a spacer sequence is located between the environmentally-sensitive linker and the nanocatalyst.

### Methods to Produce an In Vivo Sensor

Any suitable method may be used to produce an in vivo sensor described herein. In some embodiments, the method comprises incubating an environmentally-responsive linker and a reducing agent (e.g., L-glutathione (GSH) peptide) with a metal precursor solution (e.g., chloroauric acid (HAuCl₄) or chloroplatinic acid (H₂PtCl₆)), wherein the environmentally-responsive linker comprises a cysteine residue or is thiol-terminated. The environmentally-responsive linker may further comprise a functional handle. As used herein, a functional handle is a moiety (e.g., an amino acid, a protein, a chemical, or a nucleic acid) that is capable of forming a covalent bond with a cognate partner. Non-limiting examples of functional handles include a cysteine residue (e.g., which is capable of forming a bond), maleimide, pyridazinedione, a dibenzocyclooctyne (DBCO), an amine, a SpyCatcher tag, a SpyTag, a biotin, an alkyne, avidin, and an azide. Non-limiting examples of functional handle partners include a dibenzocyclooctyne (DBCO), an amine, a SpyCatcher tag, a SpyTag, a biotin, an alkyne, avidin (e.g., avidin, streptavidin, NeutrAvidin, and CaptAvidin), and an azide. As an example, a cognate partner for DBCO includes amines and vice versa. A cognate partner for SpyCatcher includes SpyTag and vice versa. A cognate partner for biotin includes avidin and vice versa. A cognate partner for azide includes alkynes and vice versa. As an example, azide and alkynes can react and allow for click chemistry. Other functional handle partners that engage in click chemistry are also encompassed by the present disclosure. See, e.g., Kolb et al., Angew Chem Int Ed Engl. 2001 Jun 1;40(11):2004-2021.

In some embodiments, a nanocatalyst is synthesized with a ratio of environmentally-responsive linker to reducing agent (e.g., GSH) of at least 1:1, 1:2, 1:3, 1:4, 1:5, 1:6, 1:7, 1:8, 1:9, 1:10, 1:15, 1:20, 1:30, 1:40, 1:50, or 1:100. In some embodiments, the ratio of environmentally-responsive linker to reducing agent (e.g., GSH) is between 1:1 and 1:5.

In some embodiments, a nanocatalyst is synthesized with a fixed ratio of reducing agent to [Metal]. In some embodiments, the ratio is at least 1:1 (e.g., 1:2, 1:3, 1:4, 1:5, 1:6, 1:7, 1:8, 1:9, 1:10, or 1:20).

In some embodiments, nanocatalyst (e.g., gold nanocluster) synthesis proceeds at an elevated temperature (e.g., at least 50° C., at least 60° C., at least 70° C., at least 80° C., at least 90° C., or at least 100° C.). In some embodiments, the incubation time is at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In some embodiments, the method results in the production of a nanoparticle (e.g., gold nanocluster) that is capped and stabilized by both the reducing agent (e.g., GSH) and an environmentally-responsive linker and exhibit both intrinsic fluorescence and peroxidase-like catalytic activity. In some embodiments, the nanoparticle (e.g. gold nanocluster) is capable of being released from the environmentally-responsive linker in vivo. In some embodiments, peroxidase-like catalytic activity is an ability to disproportionate H₂O₂.

Functional handles may be used to bind an environmentally-responsive linker (e.g., an environmentally-responsive linker that is attached to a nanocatalyst) to a scaffold. For example, the environmentally-responsive linker be linked to a functional handle (e.g., dibenzocyclooctyne (DBCO), an amine, a SpyCatcher tag, a SpyTag, a biotin, an alkyne, avidin, and an azide) and the scaffold may be linked to the cognate functional handle partner (e.g., dibenzocyclooctyne (DBCO), an amine, a SpyCatcher tag, a SpyTag, a biotin, an alkyne, avidin, and an azide). The environmentally-responsive linker and the functional handle may then be incubated together such that the functional handle can bind its cognate binding partner.

In some embodiments, the methods of the present disclosure produce nanocatalysts (e.g., catalytic nanoclusters) with high reproducibility. In some embodiments, reproducibility is a coefficient of variation between the measured catalytic activities of a nanocatalyst (e.g., catalytic nanocluster) synthesized on different days with fresh dilutions of starting materials.

In some embodiments, a high reproducibility is a coefficient of variation that is less than 50% (e.g., less than 40%, less than 30%, less than 20%, less than 10%, less than 5%, or less than 1%). In some embodiments, high reproducibility is indicated with a low coefficient of variation (e.g., a coefficient of variation of less than 10%).

### Methods to Detect Environmental Triggers In Vivo

Aspects of the disclosure relate to the surprising discovery that sensors comprising a nanocatalyst (e.g., catalytic nanocluster), are useful for detecting an environmental trigger in vivo. As an example, a sensor of the present disclosure may be used to detect in vivo enzyme (e.g., protease) activity, a particular pH, light (e.g., at a particular wavelength), or temperature in a subject.

As used herein, a biological sample is a tissue sample (such as a blood sample, a hard tissue sample, a soft tissue sample, etc.), a urine sample, saliva sample, fecal sample, seminal fluid sample, cerebrospinal fluid sample, etc. In preferred embodiments, the biological sample is a tissue sample. The tissue sample may be obtained from any tissue of the subject, including brain, lymph node, breast, liver, pancreas, colon, liver, lung, blood, skin, ovary, prostate, kidney, or bladder. The tissue from which the biological sample is obtained may be healthy or diseased. In some embodiments, a tissue sample comprises tumor cells or a tumor.

A tissue sample for use in methods described by the disclosure may be unmodified (e.g., not treated with any fixative, preservative, cross-linking agent, etc.) or physically or chemically modified. Examples of fixatives include aldehydes (e.g., formaldehyde, formalin, glutaraldehyde, etc.), alcohols (e.g., ethanol, methanol, acetone, etc.), and oxidizing agents (e.g., osmium tetroxide, potassium dichromate, chromic acid, potassium permanganate, etc.). In some embodiments, a tissue sample is cryopreserved (e.g., frozen). In some embodiments, a tissue sample is embedded in paraffin.

A sensor of the present disclosure may also be used to detect an environmental trigger (e.g., enzyme, pH, light, or temperature) in vitro. As an example, an in vitro sensor may be added to a biological sample to assess enzyme activity.

### Methods for Detecting Disease in a Subject

In some aspects, the disclosure provides methods for detecting disease (e.g., cancer, pulmonary embolism, inflammation, and infectious diseases, such as, bacterial infections, viral infections (e.g., HIV) and malaria) in a subject. As used herein, a subject is a human, non-human primate, cow, horse, pig, sheep, goat, dog, cat, or rodent. In all embodiments human subjects are preferred. In aspects of the invention pertaining to disease diagnosis in general the subject preferably is a human suspected of having a disease, or a human having been previously diagnosed as having a disease. Methods for identifying subjects suspected of having a disease may include physical examination, subject's family medical history, subject's medical history, biopsy, or a number of imaging technologies such as ultrasonography, computed tomography, magnetic resonance imaging, magnetic resonance spectroscopy, or positron emission tomography.

In some embodiments, methods described by the disclosure result in identification (e.g., detection) of a disease in a subject prior to the onset of symptoms. In some embodiments, a tumor that is less than 1 cm, less than 0.5 cm, or less than 0.005 cm is detected using methods described by the disclosure. In some embodiments, the tumor that is detected is between 1 mm and 5 mm in diameter (e.g., about 1 mm, 2 mm, 3 mm, 4 mm, or about 5 mm) in diameter. In some embodiments, a pathogen-specific enzyme (e.g., a protease) is detected (e.g., in a sample from a subject administered a sensor) during the incubation period of an infectious disease. In some embodiments, a subject with an infectious disease is contagious.

In some embodiments, the presence of an environmental trigger indicative of a disease (e.g., enzyme, pH, light, or temperature) in a subject is identified by obtaining a biological sample from a subject that has been administered a sensor as described by the disclosure and detecting the presence of a nanocatalyst (e.g., catalytic nanocluster) in the biological sample. Generally, the biological sample may be a tissue sample (such as a blood sample, a hard tissue sample, a soft tissue sample, etc.), a urine sample, saliva sample, fecal sample, seminal fluid sample, cerebrospinal fluid sample, etc.

Detection of one or more nanocatalysts in the biological sample may be indicative of a subject having a disease (e.g., cancer, pulmonary embolism, inflammation, and infectious diseases, such as, bacterial infections, viral infections (e.g., HIV) and malaria). In some instances, detection of one or more detectable markers in the biological sample is indicative of a specific stage of a disease (e.g., metastatic or non-metastatic, contagious or non-contagious, etc.). In some embodiments, detection of one or more nanocatalysts in the biological sample is indicative of a type of disease (e.g., type of cancer, type of bacterial infection, type of viral infection, or disease of a particular tissue).

In some embodiments, the limit of detection for a nanocatalyst in a biological sample is less than 100 picomoles, less than 90 picomoles, less than 80 picomoles, less than 70 picomoles, less than 60 picomoles, less than 50 picomoles, less than 40 picomoles, less than 30 picomoles, less than 20 picomoles, less than 10 picomoles, less than 9 picomoles, less than 8 picomoles, less than 7 picomoles, less than 6 picomoles, less than 5 picomoles, less than 4 picomoles, less than 3 picomoles, less than 2 picomoles, less than 1 picomole, less than 0.5 picomole, less than 0.1 picomole, or less than 0.01 picomole. In some embodiments, the limit of detection is 2 picomoles.

As described above, detection of a nanocatalyst may include detection of luminescence, fluorescence or a colorimetric assay. For example, the catalytic activity of a nanocatalyst may be detected (e.g., quantified). A nanocatalyst may be capable of promoting oxidation (e.g., capable of disproportionating H₂O₂). A non-limiting example of an oxidation assay includes assays that use a peroxidase substrate. Exemplary peroxidase substrates include chromogenic substrates (e.g., 3,3′,5,5′-Tetramethylbenzidine (TMB), 4-chloro-1-naphthol (4CN), 2,2′-azino-di-[3-ethylbenzthiazoline-6-sulfonic acid] (ABTS), 3,3′-diaminobenzidine (DAB), or a substrate is suitable for detection of HRP (e.g., in an ELISA)). In some instances, a substrate is a chromogenic, chemiluminescent, or fluorogenic substrate. Oxidized substrates may then be measured and quantified using colorimetric assays (e.g., by determining absorbance of a sample at a given wavelength), luminescence assays, fluorescence assays, and enzyme-linked immunosorbent assays (ELISAs).

### Administration

Compositions comprising any of the in vivo sensors described herein can be administered to any suitable subject. In some embodiments, the in vivo sensors of the disclosure are administered to the subject in an effective amount for detecting an environmental trigger (e.g., enzyme activity, pH, light, or temperature). An “effective amount”, for instance, is an amount necessary or sufficient to cause release of a nanocatalyst in the presence of an environmental trigger (e.g., enzyme activity, pH, light, or temperature). The effective amount of an in vivo sensor of the present disclosure described herein may vary depending upon the specific compound used, the mode of delivery of the compound, and whether it is used alone or in combination. The effective amount for any particular application can also vary depending on such factors as the disease being assessed or treated, the particular compound being administered, the size of the subject, or the severity of the disease or condition as well as the detection method. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective regimen can be planned.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The agent may comprise different types of carriers depending on whether it is to be administered in solid, liquid or aerosol form, and whether it need to be sterile for such routes of administration as injection.

Aspects of the disclosure relate to systemic administration of an in vivo sensor to a subject. In some embodiments, the systemic administration is injection, optionally subcutaneous injection. The in vivo sensors of the present disclosure may also be administered through any suitable routes. For instance, the compounds of the present invention can be administered intravenously, intradermally, intratracheally, intraarterially, intralesionally, intratumorally, intracranially, intraarticularly, intraprostaticaly, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference).

### EXAMPLES

### Example 1

### Design of Gold Nanocluster Functionalized Protease Nanosensors

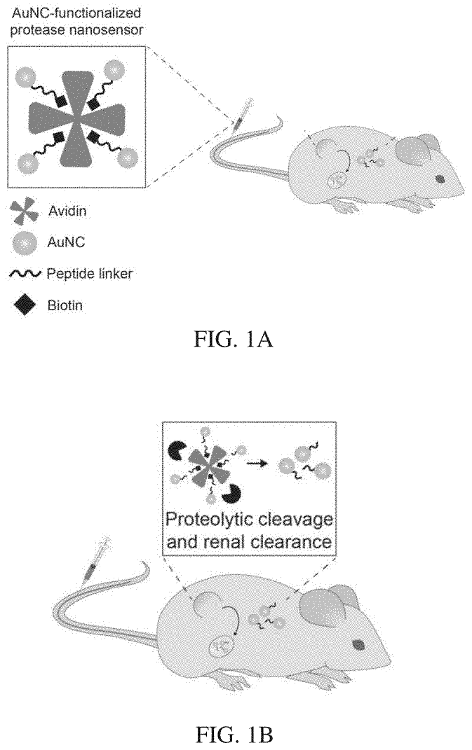

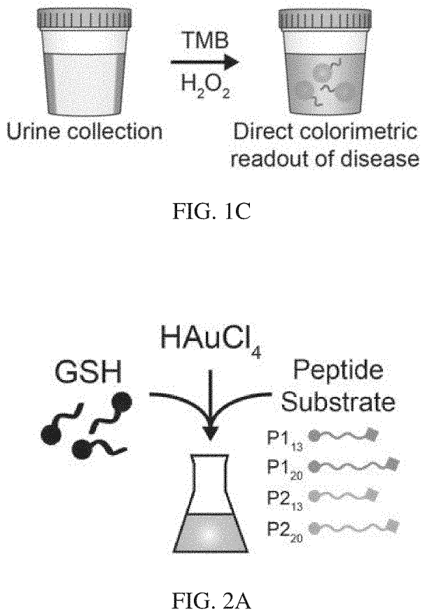

First, with the aim of designing a protease nanosensor that generates a colorimetric urinary readout, catalytic gold nanoclusters were synthesized in the renal clearance size regime and linked to protease-cleavable peptide sequences. The avidin protein analogue, neutravidin, was selected as a carrier for protease-responsive gold nanocluster reporter probes (FIGS. 1A-1C). Neutravidin protein is a deglycosylated native avidin from egg whites with a more neutral isoelectric point than avidin, and less nonspecific binding properties. This protein carrier was chosen for its efficient binding to biotinylated ligands, and broad use as a biocompatible nanocarrier for biopharmaceuticals (Jain et al., Mol. Pharm. 2017, 14, 1517-1527). In the model, the AuNC-neutravidin (AuNC-NAv) complex is intravenously administered and specifically disassembled by proteases at the site of disease. Once liberated from the avidin complex through peptide substrate cleavage, free AuNCs circulate via the bloodstream and are efficiently filtered into the urine through the kidneys due to their size. A simple colorimetric assay is performed on the urine to assess the presence of AuNCs as an indicator of disease state.

### Example 2

### Synthesis and Characterization of Peptide-Functionalized Catalytic Gold Nanoclusters

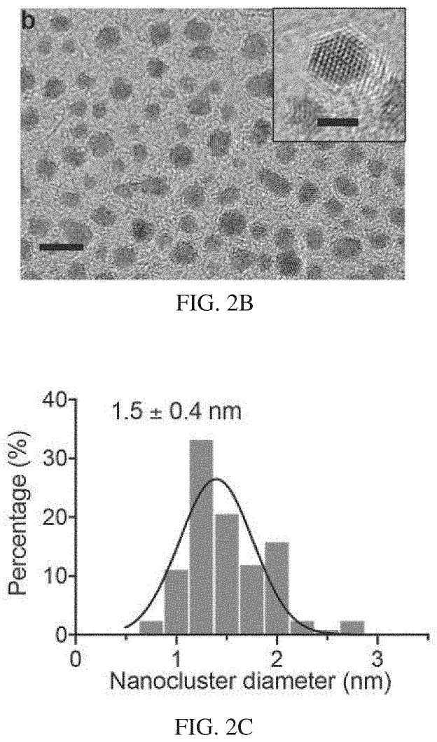

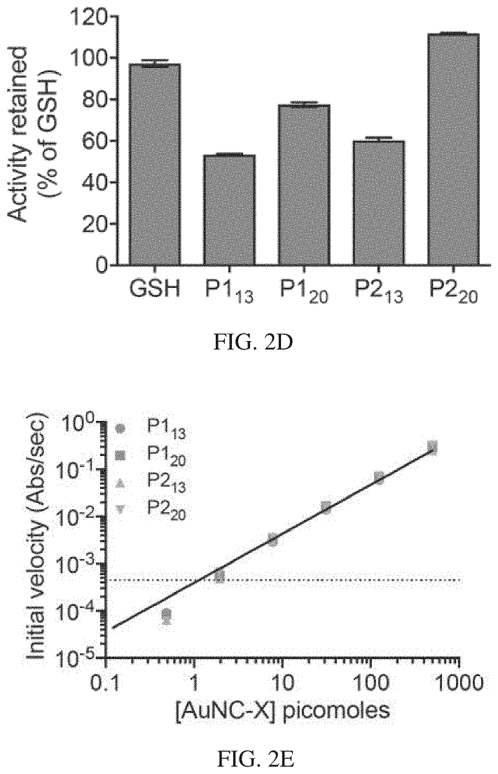

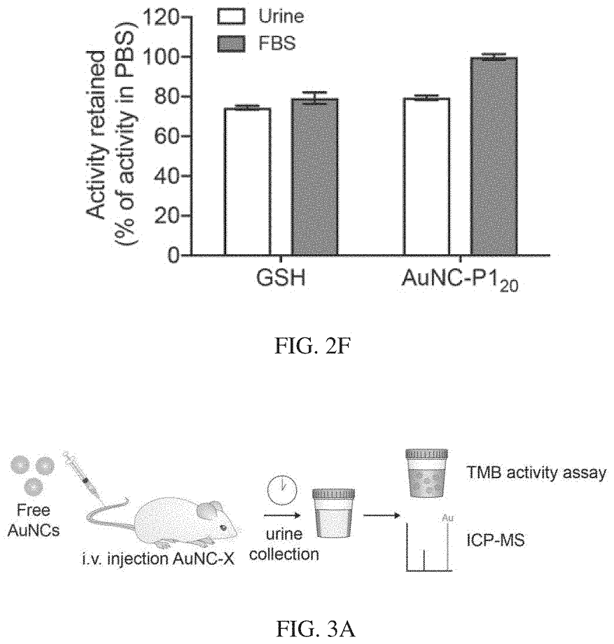

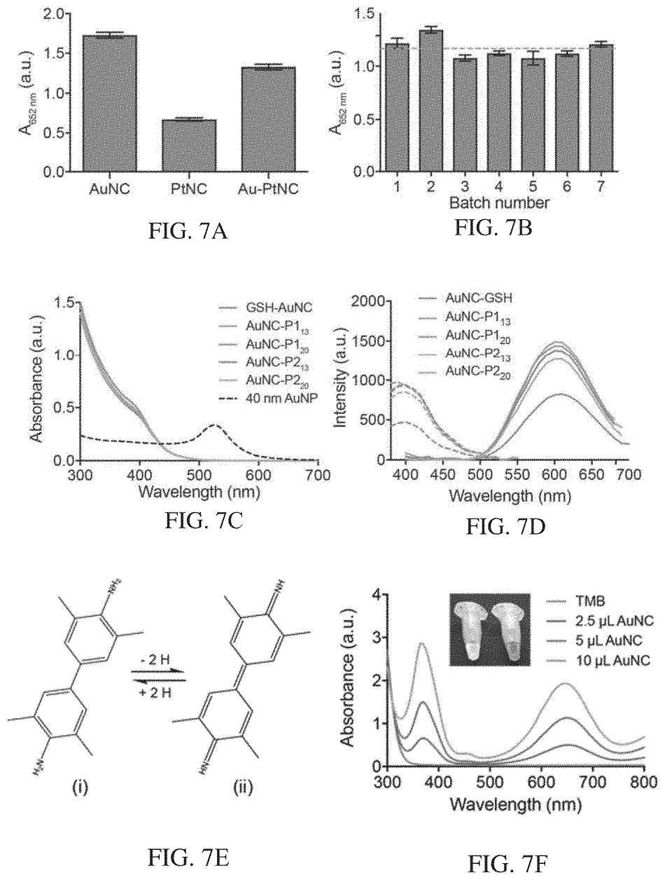

The tripeptide glutathione (GSH, γ-Glu-Cys-Gly) was used as a capping ligand for the synthesis of <2 nm diameter noble metal nanoclusters. A co-templated approach was used to synthesize gold nanoclusters, utilizing both glutathione and another thiol terminated peptide. The peptides act as both a stabilizing capping ligand and reducing agent for nanoparticle formation (FIG. 2A). Gold was selected as the core metal, as it exhibited the highest catalytic activity compared to platinum and gold-platinum bimetallic hybrid nanoclusters when synthesized with a fixed GSH:[Metal] of 1.5 (FIG. 7A). A library of protease substrate peptides was selected to template the synthesis of

AuNCs and their responsiveness to the target protease was investigated. Catalytic AuNCs were synthesized using GSH in a ratio with another thiol terminated protease-cleavable peptide sequence: P1₁₃, P1₂₀, P2₁₃, or P2₂₀, where the subscript indicates the number of amino acid residues in each sequence (Table 3 and Table 4), and AuNCs synthesized with the respective peptides are subsequently labelled AuNC-P1_{13/20} and AuNC-P2_{13/20}. In Table 4, lowercase indicates d-stereoisomer and Q indicates a quenched substrate with the FAM-CPQ2 FRET pair, where 5FAM is the fluorophore and CPQ2 is the quencher.

TABLE 3
Library of protease-cleavable thiol-terminated peptide sequences

| Substrate | Protease | | MW | Product |
|---|---|---|---|---|
| (P_{#aa}) | specificity | Sequence (↓ represents scissile bond) | (g/mol) | (g/mol) |
| P1₁₃ | Thrombin | Biotin-SGGfPR↓SGGSGGC | 1350 | 846 |
| | | (SEQ ID NO: 1) | | |
| P1₂₀ | Thrombin | Biotin-GGGSGGGSGGfPR↓SGGGGGC | 1750 | 1275 |
| | | (SEQ ID NO: 2) | | |
| P2₁₃ | MMP9 | Biotin-GGGPLG↓VRGKGGC | 1339 | 683 |
| | | (SEQ ID NO: 3) | | |
| P2₂₀ | MMP9 | Biotin- | 1739 | 1080 |
| | | GGGGGGGGGGPLG↓VRGKGGC | | |
| | | (SEQ ID NO: 4) | | |

TABLE 4
Sequences of all peptides employed in study.

| Sub- | | |
|---|---|---|
| strate | Protease | Sequence (↓ represents |
| P_{#aa}) | specificity | scissile bond) |
| P1Q | Thrombin | (5FAM)-GG fPR↓SGGGK(CPQ2)- |
| | | (PEG2)-C (SEQ ID NO: 5) |
| P1₁₃ | Thrombin | Biotin-SGGfPr↓SGGSGGC |
| | | (SEQ ID NO: 1) |
| P1₂₀ | Thrombin | Biotin- GGGSGGGSGGfPr↓SGGGGGC |
| | | (SEQ ID NO: 2) |
| P2Q | MMP9 | (5FAM)-GG PLG↓VRGKK(CPQ2)- |
| | | (PEG2)-C (SEQ ID NO: 6) |
| P2₁₃ | MMP9 | Biotin-GGGPLG↓VRGKGGC |
| | | (SEQ ID NO: 3) |
| P2₂₀ | MMP9 | Biotin-GGGGGGGGGGPLG↓VRGKGGC |
| | | (SEQ ID NO: 4) |

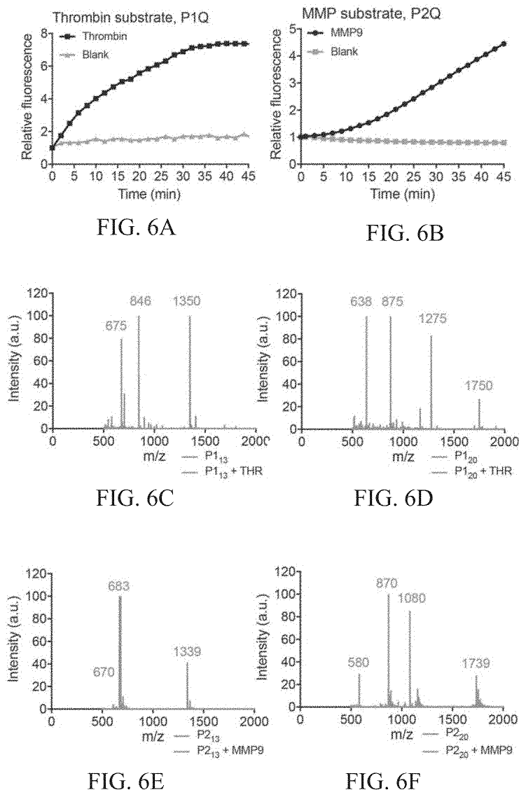

The peptide substrates used as templates for AuNC synthesis were composed of three functional domains. The core amino acid sequence is composed of the relevant enzyme recognition motif (e.g. fPRS for thrombin cleavage, and PLG for MMP9 cleavage). The criteria for peptide design also included a C-terminal cysteine residue to provide a thiol group for sequestering Au ions. Finally, the N-terminus contains a labile “click” group which allows for further site selective modification. In this work, a biotin ligand was incorporated on the N-terminus of the peptides for efficient conjugation to an avidin carrier protein. The advantage of this synthesis route to generate both luminescent and catalytic noble metal nanoclusters is the ability to incorporate responsive and functional ligands onto the surface through simple gold-thiol interactions in a one-pot synthesis. Another design consideration was the presentation of the peptide sequences bound to the surface of the AuNCs. To determine whether the protease would be sterically hindered from accessing the scissile bond when the peptide sequence is presented on the AuNC and simultaneously linked to the avidin core, longer peptides (P1₂₀, P2₂₀) were also synthesized by incorporating glycine spacers between the N-terminus and protease recognition motif. The number of amino acid residues was fixed at 6 between the C-terminal cysteine (attachment to Au surface) and protease recognition motif, and only varied the spacer arm between the biotin and scissile bond. The additional glycine residues increased the peptide length by ca. 500 Da or equivalent of PEG4 spacer (2.9 nm). The ability of the relevant protease to cleave the peptide substrate was assessed using a fluorescence dequenching assay and by verifying the mass of fragments after in vitro protease degradation using mass spectrometry. As designed, peptides P1₁₃ and P1₂₀ were cleaved specifically by thrombin, while peptides P2₁₃ and P2₂₀ were cleaved efficiently by MMP9 (FIGS. 6A-6F).

TEM of the peptide-templated AuNCs (FIG. 2B) showed that the average size (1.5±0.4 nm, FIG. 2C) was below the glomerular filtration cut-off (ca. 5 nm), making them ideally suited for kidney clearance (Yu et al., Angew. Chemie-Int. Ed. 2016, 55, 2787-2791; Ning et al., APL Mater. 2017, 5; Liu et al., J. Am. Chem. Soc. 2013, 135, 4978-4981; Soo Choi et al., Biotechnol. 2007, 25, 1165-1170. Additionally, the AuNCs produced here do not exhibit surface plasmon resonance, (typically at 520 nm, a characteristic absorption of large AuNPs) (FIGS. 7A-7D). Instead, the AuNCs exhibit molecular-like absorption and corresponding fluorescence properties with an emission peak at 600 nm, attributed to the discrete electronic state arising from their small size regime (FIGS. 7A-7D). It was also demonstrated that this synthesis method produces AuNCs with high reproducibility and low coefficient of variation (CoV) between the measured catalytic activities of AuNCs synthesized on different days with fresh dilutions of starting materials (CoV=8.5%), which is an important consideration in designing a scalable diagnostic platform (FIGS. 7A-7D).

### Example 3

### Catalytic Activity of Peptide-Templated AuNCs

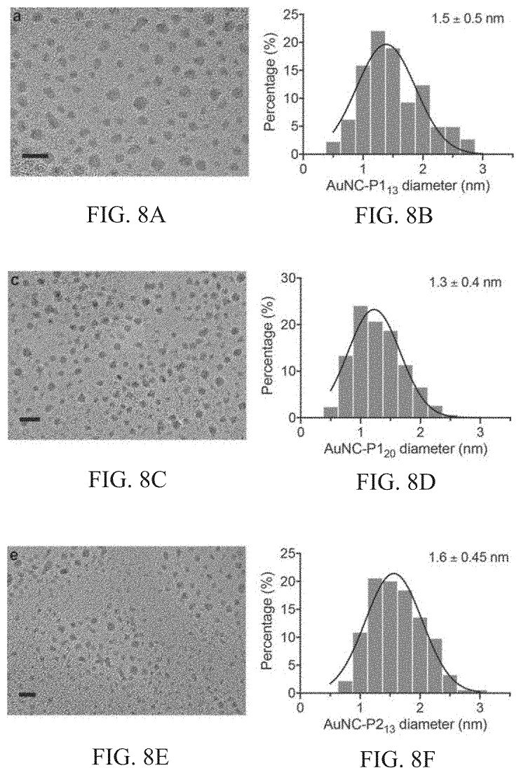

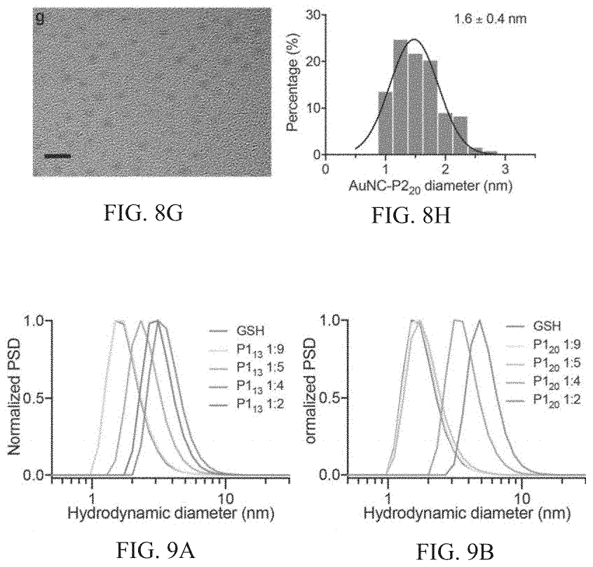

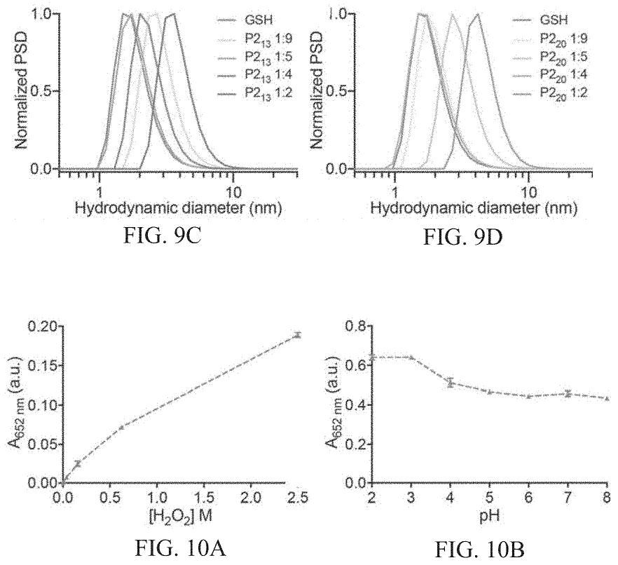

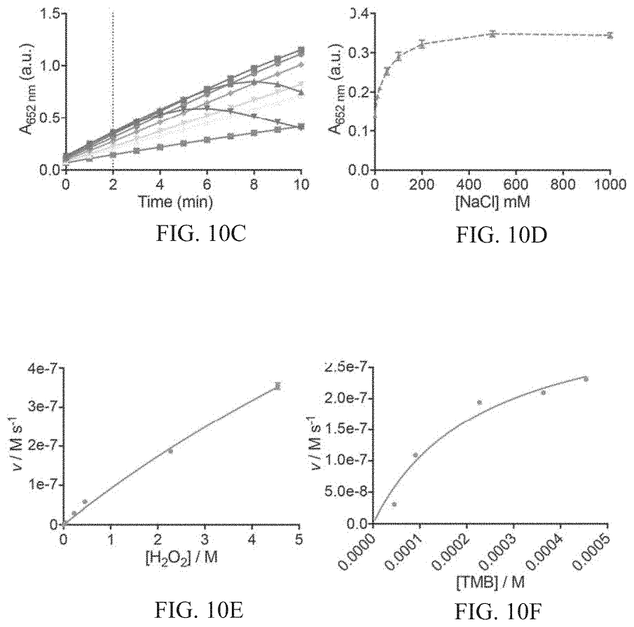

The ratio of protease-cleavable peptide substrate (P1 or P2) to glutathione in the AuNC synthesis was varied to incorporate functional handles onto the AuNC surface (P1: or P2:GSH, tested at 1:2, 1:4, 1:5, 1:9). It was confirmed that the co-peptide templated synthesis produces AuNCs by TEM and DLS (FIGS. 8A-8H and FIGS. 9A-9D). TEM size analysis shows a narrow size distribution for all batches with average diameter ca. 1.5 nm. The peroxidase-like catalytic activity of the resulting AuNCs was measured using the oxidation of TMB by H₂O₂ as a model catalytic reaction, and absorbance at 652 nm provided a colorimetric readout of AuNC activity. The catalytic activity was also analyzed using the initial rate of reaction for AuNC catalyzed oxidation of TMB. The colorimetric readout was carefully optimized to maximize signal intensity from AuNCs by varying concentration of hydrogen peroxide, pH, and concentration of sodium chloride, and measuring corresponding catalytic activity under these conditions (FIGS. 10A-10F). Colorimetric signal increased with increasing concentration of hydrogen peroxide, plateauing at ca. 2 M. As a result, PBS spiked with 2.5 M H₂O₂ was selected as the assay reaction buffer due to its neutral pH and optimal salt concentration.

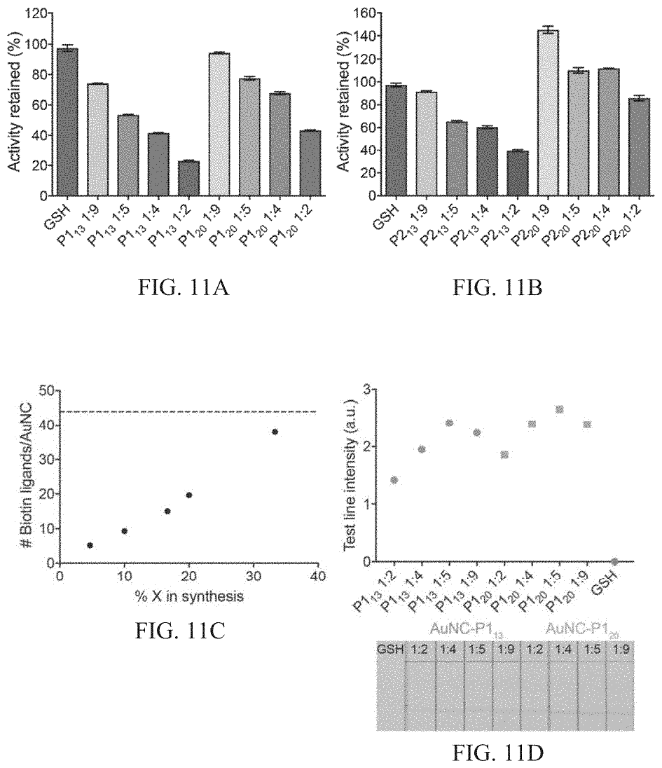

With the optimal reaction conditions set, the catalytic activity of AuNCs synthesized with the library of protease-cleavable peptide sequences was evaluated (FIG. 2D). AuNCs synthesized with a 1:5 ratio of P1 to GSH and a 1:4 ratio of P2 to GSH retained a significant amount of catalytic activity compared to AuNCs synthesized in the presence of only GSH. Activity was found to have decreased with increasing amount of P1 or P2 incorporated onto the AuNC surface during the synthesis (FIGS. 11A-11B). This decrease in activity compared to only GSH-capped AuNCs could be ascribed to the bulkier peptides replacing GSH in the synthesis. The longer peptides may block access to the AuNC surface, decreasing surface area available for interaction with substrate molecules and subsequent catalytic reactions. The differences in catalytic activity with varying peptide sequence may be attributed to variations in peptide hydrophobicity, charge, and molecular weight affecting accessibility and affinity of substrate molecules for the catalytic surface. To assess the sensitivity of the catalytic reporter probes, the catalytic activity of a dilution series of each AuNC batch in synthetic urine was measured (FIG. 2E). The limit of detection was determined to be ca. 2 picomoles, and the activity displayed a linear response over three orders of magnitude of particle concentration. The catalytic efficiency of AuNCs was quantified through apparent steady-state kinetic assays, and the data was fit to the Michaelis-Menten model to obtain kinetic parameters (FIGS. 10E-10F and Table 5). The K_{cat} of GSH-AuNCs (0.2 s⁻¹) is several orders of magnitude lower than HRP (4.0×10³ s⁻¹), which is consistent with the biological enzyme having higher specificity and affinity for the substrates than its inorganic counterpart (Gao et al., Nanotechnol. 2007, 2, 577-583). The data presented in Table 5 is exemplary and non-limiting. It is expected that specific activity ranges will vary and in some instances encompass much broader ranges than presented in Table 5. The units for each parameter are indicated after the “I” in Table 5. For example, [E] is measured in M units, Kₘ is measured in M units, Vₘₐₓ is measured in M s⁻¹ units, and K_{ca}t is measured in s⁻¹ units.

TABLE 5
Comparison of the Kinetic Parameters of Various Catalysts toward the
Oxidation of TMB by H₂O₂ (non-limiting examples of kinetic
parameters of various catalysts).

| Catalyst | [E]/M | Substrate | Kₘ/M | Vₘₐₓ/M s⁻¹ | K_{cat}/s⁻¹ |
|---|---|---|---|---|---|
| AuNC c. 1.5 nm | 1.8 × 10⁻⁶ | TMB | 2.3 × 10⁻⁴ | 3.6 × 10⁻⁷ | 0.20 |
| AuNC c. 1.5 nm | 1.8 × 10⁻⁶ | H₂O₂ | 4.5 | 1.8 × 10⁻⁶ | 0.99 |
| HRP¹ | 2.5 × 10⁻¹¹ | TMB | 4.3 × 10⁻⁴ | 1.0 × 10⁻⁷ | 4.0 × 10³ |
| HRP¹ | 2.5 × 10⁻¹¹ | H₂O₂ | 3.7 × 10⁻³ | 8.7 × 10⁻⁸ | 3.5 × 10³ |

[E] represents the catalyst concentration, Kₘ is the Michaelis constant, Vₘₐₓ is the maximal reaction velocity, and K_{cat} is the catalytic constant that equals Vₘₐₓ/[E].

There are several advantages to using inorganic AuNCs over natural peroxidases. HRP (ca. 4.5 nm) is not readily cleared through the renal filtration pathway due to its size and tendency for proteins to be reabsorbed by the tubular epithelium, so would not be feasible to use as a reporter probe in a comparable in vivo diagnostic system (Rennke et al., Kidney Int. 1978, 13, 278-288; Straus, Kidney Int. 1979, 16, 404-408; Steinman et al., J. Cell Biol. 1972, 55, 186-204; Gajhede et al., Nat. Struct. Biol. 1997, 4, 1032-1038). Additionally, as a protein, HRP would be susceptible to nonspecific degradation by endogenous proteases in vivo which would hinder activity of any cleared enzyme (Manning et al., Pharm. Res. 2010, 27, 544-575). On the other hand, AuNCs show extremely high stability in physiological environments (FIG. 2F). A key performance requirement of the AuNCs is that they retain their catalytic activity following exposure to complex environments such as patient serum, which contains ca. 7 wt % protein. Due to their small size and zwitterionic peptide capping layer, AuNCs effectively evade nonspecific protein adsorption and exhibit robust catalytic activity even after exposure to protein-rich sera environments (Soo Choi et al., Biotechnol. 2007, 25, 1165-1170). As a result, AuNCs prepared via the co-templating method retained ca. 80-90% of catalytic activity after 1 hour incubation in fetal bovine serum (FBS) or synthetic urine compared to PBS controls.

In deciding which particle platform to take forward in vivo, a system which balanced appropriate protease substrate loading with retention of activity was selected. The biotinylated protease substrate is required to form the AuNC-NAv complex, however increasing the number of P1 or P2 peptides per AuNC resulted in a decrease in activity, thus requiring a careful balance of synthesis parameters (FIGS. 11C-11D).

### Example 4

### AuNCs are cleared via the kidney and retain their catalytic activity in urine

Previous reports have investigated the renal clearance efficiency of ultra-small glutathione-protected gold nanoclusters by quantifying gold content in urine and other organs using inductively coupled plasma mass spectrometry (ICP-MS) (Zhang et al., Sci. Rep. 2015, 5, 8669 and Du et al., Nat. Nanotechnol. 2017, 12, 1096-1102). Du et al. recently highlighted the size precision of the body's response to nanoparticles by examining size-dependent glomerular filtration. In particular, they found that unlike the size dependency observed in glomerular filtration for nanoparticles larger than 2 nm, an inverse size dependency exists for particles in the sub-nanometer regime due to physical entrapment in the endothelial glycocalyx of the glomerulus, similar to the separation principle in gel filtration or size exclusion chromatography (GFC/SEC) (Du et al., Nat. Nanotechnol. 2017, 12, 1096-1102). Glutathione-capped gold particles in the 1-1.7 nm size regime clear via the kidney at ca. 50% injected dose within 24 hour post injection, whereas significantly reduced clearance is observed for clusters smaller or larger than this optimal renal clearance size regime (Du et al., Nat. Nanotechnol. 2017, 12, 1096-1102. When not bound to a carrier protein, the protease-responsive AuNCs (ca. 1.5 nm) are in the optimal size regime for efficient renal clearance.

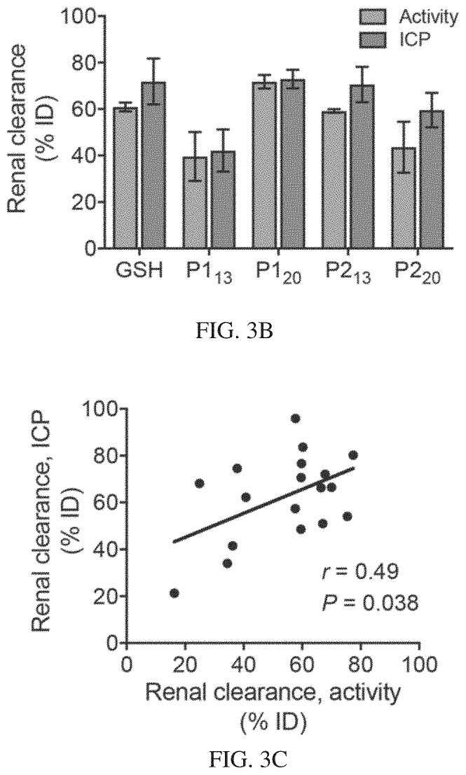

In light of their optimal size, it was necessary to determine whether the protease-cleavable AuNCs renally cleared, and whether their catalytic activity was retained after in vivo interrogation of healthy mice. The high physiological stability and retention of AuNC catalytic activity after exposure to serum and urine offered a unique opportunity to non-invasively measure AuNC clearance using both intrinsic catalytic activity and gold content with ICP-MS (FIG. 3A). AuNC renal clearance was determined by intravenous (i.v.)

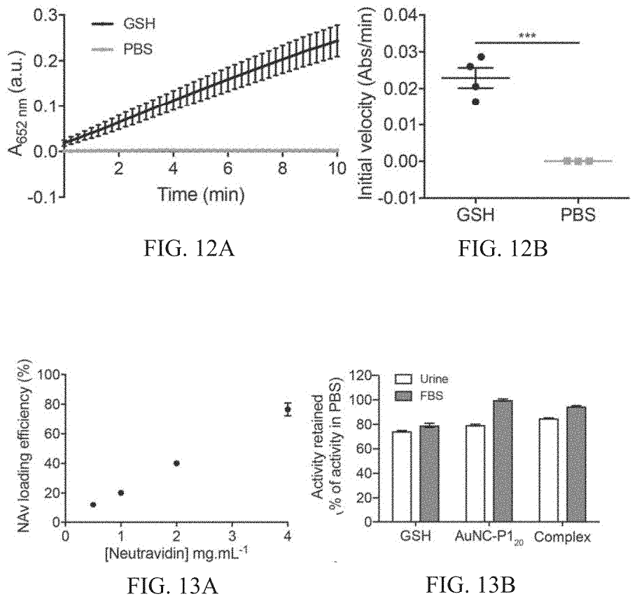

injection of AuNCs into the tail vein of healthy mice (200 μL, 10 μM particle concentration), collecting urine 1 hour post injection (p.i.), and performing both the catalytic activity assay on the collected urine and ICP-MS analysis on the same urine samples to quantify gold content. To determine renal clearance efficiency, both the catalytic activity and the gold content of AuNCs spiked into urine were used to generate a calibration curve of the injected dose. This was then used to compare activity and gold content of cleared urine. This in vivo renal clearance study showed that up to 80% of the injected dose of functionalized AuNCs left the body via this route and retained their catalytic activity in urine (FIG. 3B). Urine samples were also digested using aqua regia and ICP-MS was used to corroborate the catalytic activity assay. Gold content indicated again that renal clearance efficiency of up to 80% on the same urine samples. Encouragingly, the catalytic activity assay and ICP-MS results showed a positive correlation (Pearson correlation coefficient=0.49). The positive correlation means the results of colorimetric assay could be semi-quantitative if correlated with estimated cleared AuNCs using gold content in ppb from ICP-MS when normalized to gold content in the injected dose. The advantage of a dual readout means that catalytic activity assay could be used for a quick (<30 min) assessment of AuNC presence in urine, and ICP-MS can provide a high sensitivity readout of Au content on a longer time scale (1-3 hours due to sample digestion and preparation). As a control, urine from mice injected with PBS was analyzed using the catalytic activity assay to ensure no endogenous peroxidase activity in collected urine (FIGS. 12A-12B).

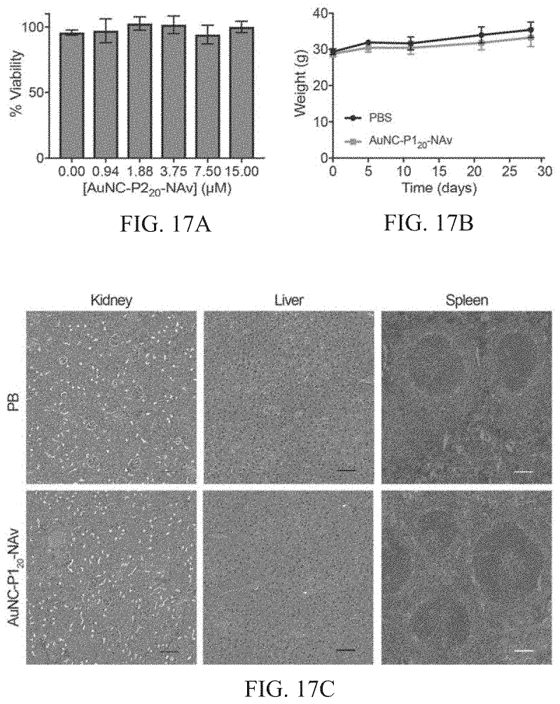

The biocompatibility of glutathione capped AuNCs has been reported. The toxicological responses of AuNC-NAv complexes were further investigated by examining the pathology of the mice. No significant changes in weight loss (2 weeks p.i.) and no evidence of fibrosis were found, suggesting that protease-cleavable AuNC-NAv complexes did not induce significant systemic liver, kidney, or spleen toxicity, relative to the PBS control (FIGS. 17A-17C). Therefore, the results successfully demonstrated that catalytic activity of AuNCs can be measured directly in cleared urine.

### Example 5

### Engineered AuNC Nanosensor Complexes are Sensitive to Protease Activity

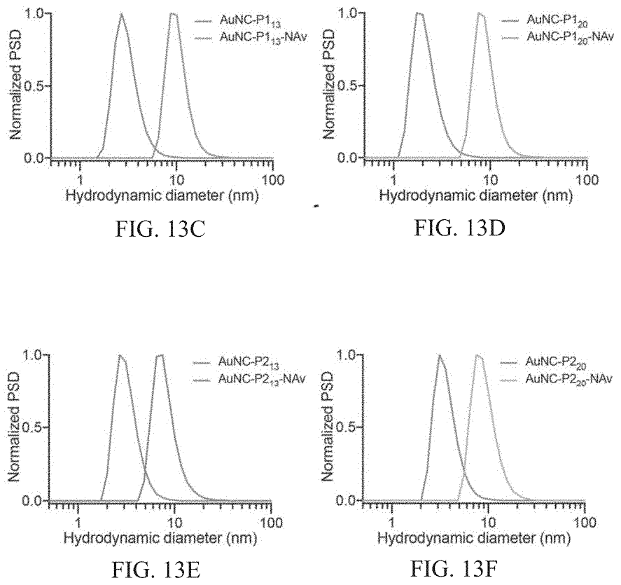

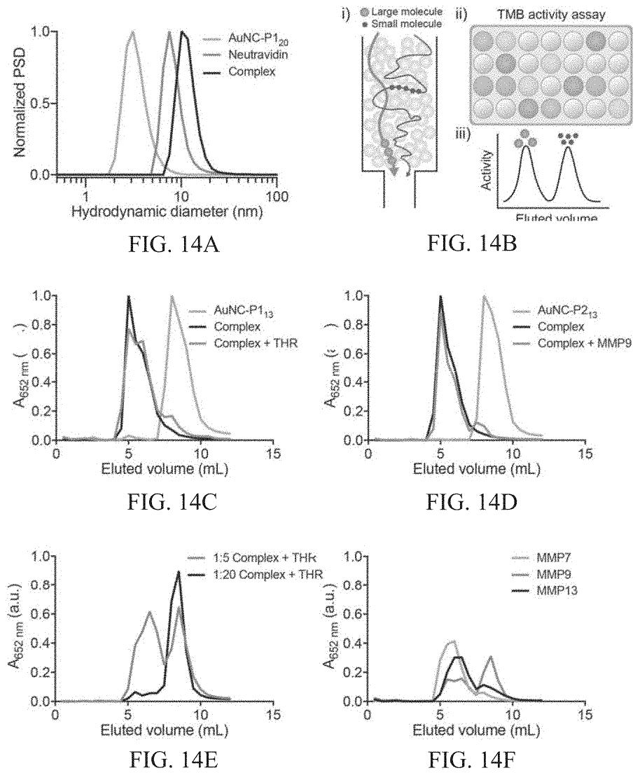

Next, it was sought to confirm whether peptides simultaneously coupled to the AuNC and avidin scaffold could still be cleaved by proteases. Biotin functional handles were used on the protease substrate-modified AuNCs to tether it to a neutravidin carrier protein to assemble a complex that is larger than the glomerular filtration cut-off (ca. 5 nm) (Longmire et al., Nanomedicine (Lond) 2008, 3,703-717; Deen et al., Am J Physiol Ren. Physiol 281 2001, 36, F579-F596; Soo Choi et al., Biotechnol. 2007, 25, 1165-1170; Du et al., Nat. Nanotechnol. 2017, 12,1096-1102. The non-clearable nanosensors were designed such that upon interaction with the relevant disease-associated protease, the complex is disassembled, and liberated AuNCs can be subsequently filtered into the urine. DLS was used to monitor the size of the free AuNCs, neutravidin carrier protein, and assembled AuNC-NAv complex (FIGS. 13A-13F and FIGS. 14A-14F), with hydrodynamic sizes ca. 2 nm, 8 nm, and 12-13 nm, respectively. The AuNC-NAv complexes show comparable physiological stability to AuNCs alone (FIGS. 13A-13F). Additionally, complexes were more efficiently formed when protease-substrate functionalized AuNCs were incubated with high concentrations of avidin protein (FIGS. 13A-13F), resulting in ca. 1-2 AuNCs loaded on each avidin carrier.

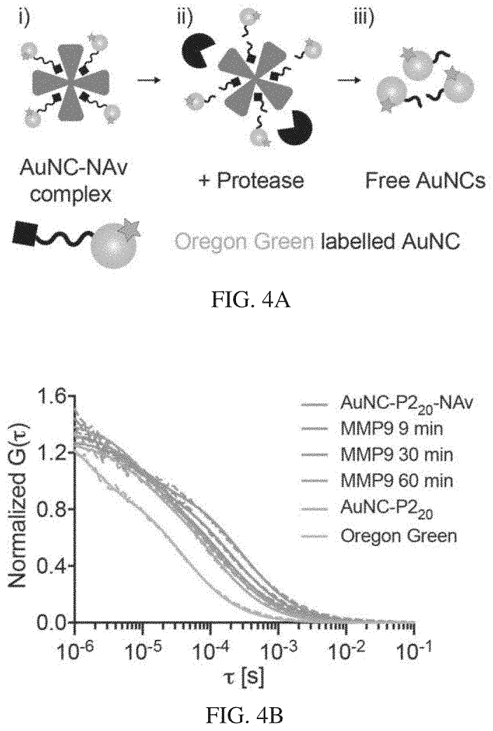

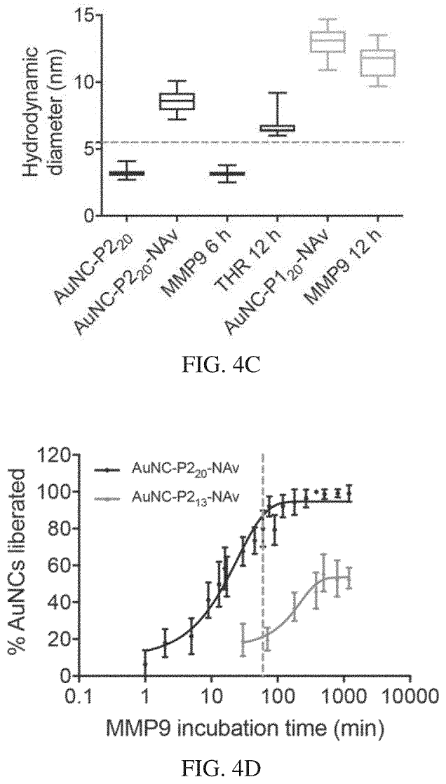

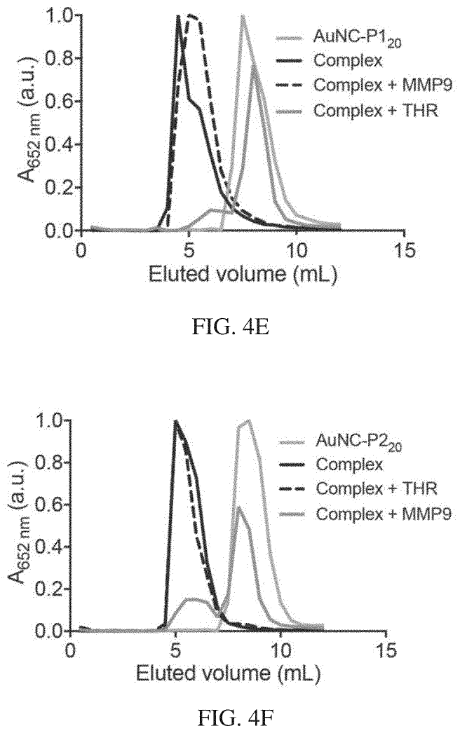

To explore the kinetics of protease cleavage of AuNC-NAv complexes using a single-molecule detection method, fluorescence correlation spectroscopy (FCS) was employed (Magde et al., Phys. Rev. Lett. 1972, 29, 705-708; Rigler et al., Eur. Biophys. J. 1993, 22, 169-175; Rigler et al., J. Am. Chem. Soc. 2006, 128, 367-373). FCS is a correlation analysis of temporal fluctuations of fluorescence intensity of fluorescent particles in a small observation volume, giving insight into the diffusion behavior and concentration of detected particles. The technique is especially useful in monitoring binding or cleavage events by analyzing changes in diffusion rates over time. For example, free fluorescently labelled AuNCs exhibit faster diffusion rates than AuNCs which are complexed to a neutravidin core. Therefore the rate of diffusion of free AuNCs or AuNC-NAv complexes can be monitored over time in the presence of enzymes to analyze the kinetics of cleavage. For FCS analysis, AuNC batches were labelled with Oregon Green fluorescent dye (at the free amino group of GSH) and assembled into complexes with the neutravidin core (FIG. 4A). FCS was used to analyze disassembly of the fluorescently labelled complex in the presence of an enzyme. In the measurement, labeled particles diffuse through the detection volume, producing a fluctuating fluorescence signal which is subjected to an autocorrelation algorithm yielding a correlation curve, G(τ), which shows the mobility of the particles. The diffusion time of the particles, τ_{D}, can be estimated from the inflection of the decay of the correlation curve. To calculate the percentage of AuNCs cleaved from the AuNC-NAv complex, the stocks of free AuNCs and complexes were first fit using one component fits to obtain diffusion time for the pure components. Second, samples incubated with enzymes were fitted with two component fits (G_{2comp}(τ)) with one component fixed to pure cluster diffusion (τ₁) and the other fixed to pure complex diffusion (τ₂) to yield the fraction of free clusters (F₁), which is equivalent to the fraction cleaved.

By inspection, it is clear from correlation curves that the free dye, free AuNCs, and AuNC-NAv complex diffused at different rates (FIG. 4B). After enzyme incubation, the diffusion rates of the AuNC-NAv complex shifted toward the rate of the free AuNCs over time. The hydrodynamic diameter could be calculated from the diffusivity using Stokes-Einstein equation (FIG. 4C). From the size analysis, the AuNC-P2₂₀-NAv complex was completely disassembled within 6 hours MMP9 incubation. Encouragingly, the size of the complex did not significantly change when incubated with an off-target enzyme, in this case thrombin. Additionally, the size of the thrombin cleavable complex, AuNC-P1₂₀-NAv, does not change when incubated with the off-target enzyme MMP9, which is relevant for future in vivo control studies (FIG. 4C).

A significant difference in cleavage kinetics was observed for AuNC-P2₁₃-NAv and AuNC-P2₂₀-NAv complexes upon interaction with MMP9 (FIG. 4D). For the AuNC-P2₁₃-NAv complex, the percentage of cleaved AuNCs with time was linear over the first 500 minutes of MMP9 incubation, whereas the AuNC-P2₂₀-NAv was linear over just the first 16 minutes of enzyme incubation. The linear regions were analyzed by linear regression, and the rates of cleavage were calculated. MMP9 exhibited a rate of 3% AuNC cleaved per minute toward the AuNC-P2₂₀-NAv complex, while the rate was only 0.08% AuNC cleaved per minute toward the AuNC-P2₁₃-NAv complex. The complex formed of the longer linker was cleaved at a rate ca. 40 times faster than the shorter linker. The difference in enzyme kinetics could be attributed to the difference in linker length, and subsequently increased accessibility of the enzyme to the scissile bond. For in vivo studies, urine was collected 1 hour post injection with the complex. To design a biologically relevant in vitro experiment, the amount of cleaved AuNCs was calculated over the first 1 hour of enzyme incubation. FCS results show that in the presence of biologically relevant enzyme concentrations (50 nM MMP9), significant cleavage is observed for AuNC-P2₂₀-NAv complexes, where 80% AuNCs are cleaved within the first hour of incubation with MMP9. In the same time frame, only 20% AuNCs are cleaved from the AuNC-P2₁₃-NAv complex.

Proteolytic cleavage of AuNC complexes was further characterized in vitro by incubating complexes with recombinant protease and using gel filtration chromatography to separate cleavage products by size (FIGS. 4E-4F). Comparisons have been drawn between the glomerular filtration of sub-nanometer AuNCs and separation in SEC/GFC, where larger molecules renally clear/elute faster than smaller ones (Du et al., Nat. Nanotechnol. 2017, 12, 1096-1102). For its biological relevance, a GFC protocol was developed to separate cleavage products by size and monitor in vitro protease cleavage with a catalytic activity readout. AuNC complexes functionalized with the longer thrombin-responsive and MMP-responsive substrates were efficiently disassembled in vitro (FIGS. 4E-4F). Disassembly of the complexes was monitored by measuring the catalytic activity of column fractions when AuNC-complex, free AuNCs, and AuNC-complex pre-incubated with recombinant protease were eluted through a chromatography column (FIG. 14B). When catalytic activity is plotted as a function of eluted volume, a clear peak is associated with each cleavage product. The larger AuNC complexes eluted at 5-6 mL, while the smaller free AuNCs eluted at 7-9 mL. After incubation with the relevant enzyme, AuNC complexes exhibited a peak in absorbance overlapping with the free AuNCs, suggesting cleavage by the enzyme liberated the AuNCs resulting in a smaller cleavage product. For the chosen incubation times and enzyme concentrations, a small peak associated with the original complex was also present, suggesting not all AuNCs were liberated from the complex in the time frame of the experiment. Because the synthesis requires more than one biotinylated protease substrate per AuNC to form the complex, it is possible that not every cleavage event resulted in liberation of an AuNC. The extent of cleavage of the AuNC complex under different conditions could be quantified by analyzing the area under the curve associated with each cleavage product (Table 6). To determine whether varying linker length will affect cleavage rates for enzyme incubation during the same time frame, long and short sequences for both MMP and thrombin cleavable complexes were compared. It was found that under same conditions, complexes formed from shorter sequences exhibited 15-20% cleavage, whereas longer peptide linker exhibited 75-90% cleavage, suggesting the scissile bond was more accessible to the enzyme in this configuration in agreement with the FCS results (FIGS. 14C-14D).

TABLE 6
Quantification of AuNC cleavage products from in vitro gel filtration
chromatography assays.

| | % Cleavage (free | |
|---|---|---|
| | AuNC fraction: 7- | Figure |
| AuNC-complex | 12 mL) | reference |
| AuNC-P1₁₃-NAv + THR (12 h) | 21.8% | FIG. 14C |
| AuNC-P1₂₀-NAv + THR (12 h) | 90.2% | FIG. 4E |
| AuNC-P1₂₀-NAv + MMP9 (12 h) | 7.1% | |
| AuNC-P2₁₃-NAv + MMP9 (12 h) | 15.2% | FIG. 14D |
| AuNC-P2₂₀-NAv + MMP9 (12 h) | 75.1% | FIG. 4F |
| AuNC-P2₂₀-NAv + THR (12 h) | 5.5% | |
| AuNC-1:20-P1₂₀-NAv + THR (1 h) | 89.9% | FIG. 14E |
| AuNC-1:5-P1₂₀-NAv + THR (1 h) | 49.6% | |
| AuNC-P2₂₀-NAv + MMP7/9/13 (12 h) | MMP7: 12.1% | FIG. 14F |
| | MMP9: 54.9% | |
| | MMP13: 29.8% | |

The specificity of the peptide substrates was explored for the proteases of interest by incubating the complexes with off-target proteases and evaluated cleavage using GFC. While several enzymes in the MMP family share similarities in primary structure and ability to cleave ECM components, the PLG domain is commonly reported as the substrate recognition profile more specifically associated with cleavage by MMP9 over MMP7 and MMP13, and was originally designed based on the consensus of the collagen cleavage site by MMPs (Eckhard et al., Matrix Biol. 2016, 49, 37-60; Kridel et al., J. Biol. Chem. 2001, 276, 20572-20578; Fields et al., Methods Mol. Biol. 2001, 495-518). Nonspecific protease cleavage was examined by incubating the MMP9-responsive complexes with a fixed concentration of MMP7, MMP13, and MMP9. MMP9 cleaved most robustly, but there was some cleavage by MMP13, and very low cleavage by MMP7 (FIG. 14F). There was both lower specific and nonspecific cleavage for the shorter linker complex (AuNC-P2₁₃-NAv) when incubated with MMPs, suggesting the possibility of tailoring specificity of cleavage site by altering accessibility to the scissile bond. Nonspecific cleavage was further investigated by incubating AuNC-P1₂o-NAv with MMP9, and AuNC-P2₂₀-NAv with thrombin (swapping enzymes with the relevant models) and observed extremely low background cleavage for an off-target enzyme (FIGS. 4E-4F). Finally, whether there was an effect of biotinylated-protease substrate loading per AuNC on the rate of cleavage over short time frames was explored. It was determined whether reducing the number of biotin ligands on the AuNC surface would have a higher probability of an enzyme cleavage event specifically liberating an AuNC from the complex, rather than cleaving off peptides that are not actively tethering the AuNC to the core. This effect was seen, where for AuNCs with only 5 biotin ligands per cluster 40% more cleavage was observed than for AuNCs presenting 20 biotin ligands per cluster for 1 hour incubation with 60 nM thrombin (FIG. 14E). However, forming the AuNC-NAv complex is over twice as efficient when the AuNCs present 20 biotin ligands per AuNC, so this loading was maintained for the current study. Future work will explore the effects of further altering presentation of ligands and capitalizing on multivalent binding to the protein carrier to introduce logic gates to improve specificity.

### Example 6

### AuNC Nanosensors Enable Colorimetric Urinary Detection of Disease

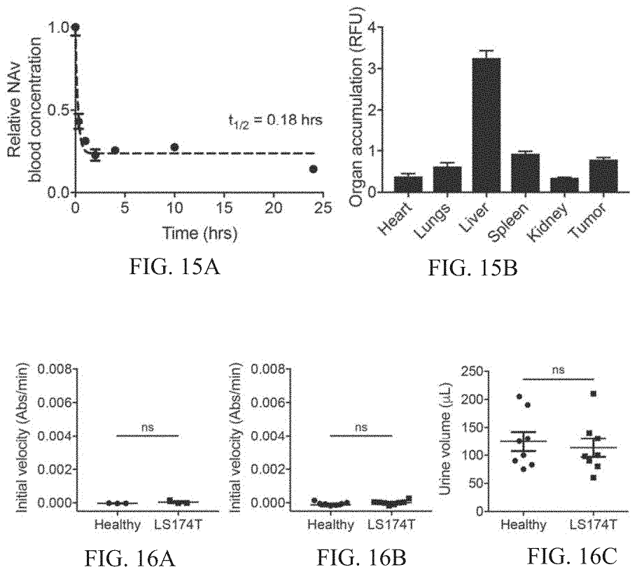

After confirming successful cleavage by recombinant proteases in vitro, it was sought to apply the protease-responsive AuNC platform to in vivo disease detection using the colorimetric urinary readout. The pharmacokinetics of the neutravidin carrier was first characterized in terms of blood half-life and accumulation in organs and tumor xenografts of the human colorectal cancer cell line LS174T 1 hour p.i. (FIGS. 15A-15B). Based on the measured blood half-life of the complex and the degree of tumor accumulation 1 hour p.i., 1 hour p.i. was selected as the time point for urine collection.

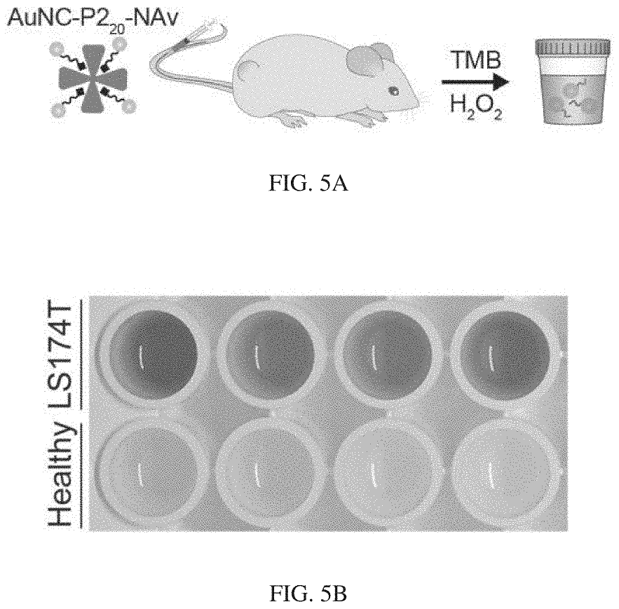

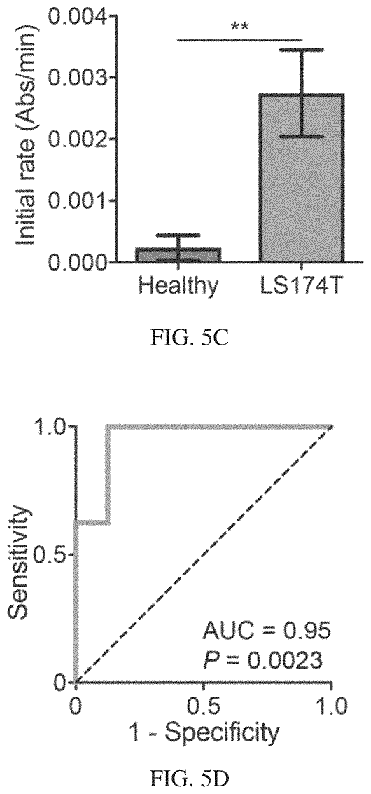

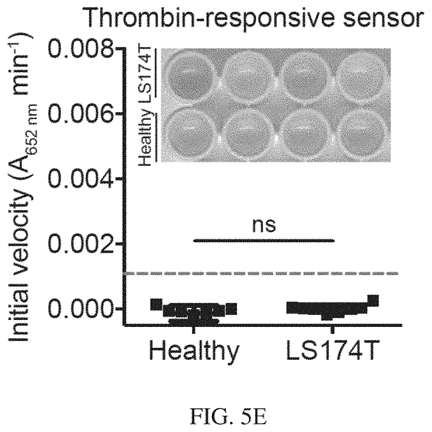

For in vivo tumor experiments, mice bearing flank xenografts of the human colorectal cancer cell line LS174T, which secretes MMP9 (Warren et al., Proc. Natl. Acad. Sci. U.S.A. 2014, 111, 3671-3676), and healthy control mice were intravenously injected with MMP-responsive AuNC-P2₂₀-NAv nanosensors (FIG. 5A). Urine was collected from mice 1 hour p.i., and catalytic activity assay was run on 25 μL sample of cleared urine. A clear blue color developed in collected urine samples containing AuNCs due to the oxidation of TMB peroxidase substrate (FIG. 5B). In the flank tumor model, a mean urinary signal increase of approximately 10-fold was found in tumor-bearing mice relative to healthy mice, as measured by the direct colorimetric readout and initial rate analysis (Abs/minute) of cleared AuNC catalytic activity in collected urine (FIG. 5C). To determine the diagnostic accuracy of the colorimetric assay, the frequency of true positives (sensitivity) and false positives (1—specificity) was assessed by receiver operating characteristic (ROC) curves (FIG. 5D). ROC curves characterize the predictive power of a biomarker by returning the area under the curve (AUC) as a metric, with a baseline AUC of 0.5 representing a random biomarker classifier. By ROC analysis, the colorimetric assay was highly accurate and discriminated the presence of colorectal cancer xenografts with an area under the curve (AUC) of 0.95 (P=0.0023).

Having established that the MMP-responsive AuNC nanosensors could discriminate between tumor-bearing and healthy mice, it was necessary to verify that the urinary signal from tumor-bearing mice was proteolytically driven. To ensure that the signal was not coming from endogenous peroxidase activity, tumor-bearing and healthy control mice were injected with PBS, urine was collected 1 hour p.i., and catalytic activity assay was performed on a 25 μL sample of cleared urine. No catalytic development was observed in urine from both tumor-bearing and healthy mice, suggesting a lack of any components with endogenous peroxidase activity (FIG. 16A).

Finally, to demonstrate the specificity of the MMP-responsive AuNC-P2₂₀-NAv nanosensors, AuNC-P1₂₀-NAv (thrombin-responsive complexes) were injected into tumor-bearing mice. In contrast to the results following administration of the MMP-responsive complexes which showed high colorimetric signal in urine, the thrombin-responsive complexes did not show any significant colorimetric signal in urine from tumor-bearing mice compared to healthy controls (FIG. 16B). This demonstrates that in the tumor model, the AuNC-P2₂₀-NAv nanosensors are specifically disassembled through interaction with MMPs at the disease site or through interactions with circulating MMPs. Taken together, these results demonstrate that the AuNC-nanosensor complexes respond to disease-specific proteolytic activity in vivo and enable a direct colorimetric readout of disease state, as evidenced by highly accurate discrimination in a flank tumor model of human colorectal cancer.

Here, a modular approach for rapid colorimetric detection of disease state has been developed. A library of ca. 1.5 nm catalytic gold nanocluster probes was synthesize and modified with protease substrates, which are responsive to MMP9 and thrombin, enzymes upregulated in tumor and pulmonary embolism microenvironments, respectively. The peptide-templated AuNCs were demonstrated to be efficiently filtered through the kidneys and excreted into the urine. Additionally, the AuNCs retained activity in physiological environments and can be used as a colorimetric indicator. The AuNC probes were assembled into larger complexes, which were disassembled in response to specific proteases. Finally, MMP-responsive AuNC complexes were deployed in vivo in a colorectal cancer mouse model and successfully detected AuNCs in urine from tumor bearing mice with a facile colorimetric readout.

While gold nanoparticles are widely used as biocompatible fluorescence and X-ray contrast imaging agents in vivo, it is shown here that through rational surface modification, nanosensors that exploit gold nanoparticle catalytic activity in vivo can be engineered as a disease indicator. Considering the similarity of rodents and humans in terms of pore sizes of the glomerular filtration membrane (Du et al., Nat. Nanotechnol. 2017, 12, 1096-1102), these results will also open new opportunities for developing translatable responsive and catalytic nanomaterial diagnostics for a range of diseases in which proteases can be used as biomarkers.

These results demonstrate that catalytic gold nanoclusters can be exploited for in vivo biosensing applications, specifically non-invasive disease detection based on a simple and sensitive colorimetric urinary assay. A novel system for rapid disease detection that requires minimal equipment and that can be read by the naked eye in less than 1 hour was reported. This approach is envisioned to be applicable in low-resource settings for rapid detection of a diverse range of disease-associated proteases. Because a versatile and modular platform that can be easily translated for detection of other proteases was designed, the plan is to expand the detection to other relevant diseases that would benefit from PoC detection, including early detection of HIV and malaria.

### Example 7

### Materials and Methods

### Materials

All chemicals were purchased from Sigma-Aldrich unless otherwise stated. Milli-Q water (18.2 MΩ.cm) was used in all the experiments.

### Solid Phase Peptide Synthesis

Peptides were synthesized manually on Rink amide resin using standard fluorenyl methoxycarbonyl (Fmoc) chemistry. The Fmoc protecting group was removed from the resin by incubating with piperidine/DMF (20:80) for 2×10 minutes. Fmoc-protected amino acids were activated with 4 molar equivalents of the Fmoc protected amino acids, 3.95 molar equivalents of N,N,N′,N′-Tetramethyl-O-(1H-benzotriazol-1-yl)uronium hexafluorophosphate, and 6 molar equivalents of diisopropylethylamine in DMF. The coupling solution was added to the resin and the coupling reaction was allowed to proceed for three hours. Peptides were cleaved in trifluoroacetic acid/triisopropylsilane/H20 (95:2.5:2.5) containing DTT for four hours. The solvent was removed in vacuum and the peptide was precipitated in cold ether. The crude products were further purified using reversed phase preparative high-performance liquid chromatography (Shimadzu) in an acetonitrile/water gradient under acidic conditions on a Phenomenex C18 Gemini NX column (5 micron pore size, a 110 A particle size, 150×21.2 mm). Molecular weight of peptide library was verified using liquid chromatography-mass spectrometry (LC-MS, Agilent Technologies).

### AuNC Synthesis

Synthesis and purification of peptide capped AuNCs followed published procedures with modifications outlined below (Zhang et al., Sci. Rep. 2015, 5, 8669). Briefly, freshly prepared aqueous solution of gold(III) chloride trihydrate aqueous solution (HAuCl₄, 20 mM, 100 μL) was mixed with 750 μL deionized water in an Eppendorf tube, followed by fast addition of L-Glutathione reduced (GSH, 20 mM) and either peptide P1 or P2 (20 mM) so that final peptide content was fixed at a total volume of 150 μL in varying ratios of P1/P2:GSH at 25° C. The reaction mixture was heated to 70° C. under gentle stirring (500 rpm) for 24 hours. The reaction mixture changed from yellow to colorless within minutes and then turned pale yellow over ca. 12 hours, indicating first reduction of Au

(III) to Au (I) by the thiol group of the peptides, followed by the reduction of Au(I) thiolate complexes to Au(0) atoms over time assisted by the favorable reduction kinetics at the elevated reaction temperature (Luo et al., J. Am. Chem. Soc. 2012, 134, 16662-16670 and Yu et al., ACS Nano 2012, 6, 7920-7927).After a 24 hour synthesis, the resulting AuNC solution exhibits both orange luminescence and simultaneous peroxidase-like activity. The AuNCs could be stored at 4° C. for >6 months with negligible changes in optical or catalytic properties. The as-prepared AuNCs were purified through centrifugal ultrafiltration (Amicon Ultra centrifugal filter units Ultra-15, MWCO 10 kDa, Sigma) and buffer exchanged into phosphate buffered saline (PBS, pH 7.2). During ultrafiltration, the AuNCs were collected in the concentrate in the filter device, while any unbound peptide was collected in the filtrate. After purification, AuNCs were resuspended in PBS (20 μM) and sterile filtered (Millex-GV Filter, Millipore, 0.22 μm).

The number of biotinylated ligands per AuNC was calculated by measuring biotin concentration in the filtrate from AuNC purification above, and subsequently subtracting this value from the starting concentration of biotinylated peptide in the synthesis. Biotin concentration in the filtrate was quantified using the Pierce Biotin Quantitation kit following manufacturer's instructions (Thermo Fisher) without any modifications. The molarity of biotin in the sample was calculated using Beer Lambert's Law: A=cbC, where A is the absorbance of the sample; ε is the extinction coefficient at a particular wavelength, for HABA/avidin at 500 nm it is 34000 M⁻¹ cm⁻¹; b is the path length in cm, ca. 0.5 cm for 200 μL volume in a 96 well plate; and C is concentration in mol/L.

### Characterization of Nanoparticles

Dynamic light scattering (DLS, Zeta Sizer Nanoseries, Malvern Instruments, Ltd.) was used to characterize the hydrodynamic radius of nanoparticles. Absorption measurements were recorded on a SpectraMax M5 multimode microplate reader (Molecular Devices, Ltd.). For electron microscopy characterization, samples were drop-casted onto carbon-coated copper grids (Electron Microscopy Sciences), and TEM imaging was performed using a JEOL 2100F operating at 200 kV. For preparation of TEM samples, AuNC samples were first desalted (Zeba Spin Desalting Columns, 7K MWCO, Sigma) and 5 μL desalted sample was dropped onto the grid, allowed to incubate for 5 min, and subsequently wicked with filter paper and dried overnight before imaging.

Evaluation of Peroxidase-Like Activity For stability and catalytic activity of AuNCs in physiological environments, AuNCs (20 μM, 50 μL) were incubated with PBS (50 μL), synthetic urine (Surine Negative Urine Control, Sigma), or fetal bovine serum (FBS, Gibco) for 1 hour at 37° C. followed by five-fold dilution in water. For the activity assay, 50 uL of each sample was added to a 96-well plate (Corning, UK) followed by 150 uL chromogenic substrate solution: 1-Step Ultra TMB ELISA Substrate Solution (Thermo Scientific) spiked to a final concentration of 4 M hydrogen peroxide (30% (w/w), Sigma). The absorbance of the reaction solution at 652 nm was monitored up to 25 minutes after the addition of substrate, corresponding to oxidation of TMB by H₂O₂.

For limit of detection assays, in a 96-well plate, synthetic urine (25 μL) was mixed with AuNCs (25 μL varying concentrations, diluted in PBS), 5 M H₂O₂ (100 μL), and 1-Step Ultra TMB ELISA Substrate Solution (100 μL). Absorbance at 652 nm was measured every 20 seconds for 10 minutes, and linear regression was used to calculate the slope (Absorbance/sec) over the first 150 seconds.

### Steady-State Kinetic Assays

Steady-state kinetic assays were carried out at room temperature in a 96-well plate with 220 μL solution with estimated path length (l) of 0.5 cm. 25 mM NaOAc/HOAc solution (pH 4.0) was used as the reaction buffer. For kinetic assays varying 3,3′,5,5′-Tetramethylbenzidine (TMB), AuNCs (20 20 μL) were mixed with TMB (10 μM to 1 mM, 100 μL) and H₂O₂ (5 M, 100 μL). For kinetic assays varying H₂O₂, AuNCs (20 20 μL) were mixed with H₂O₂ (0 to 10 M, 100 μL) and TMB (1 mM, 100 μL). After addition of substrates (TMB and H₂O₂) in the buffer system containing AuNCs, the absorbance of the reaction solution at 652 nm of each sample was immediately measured as a function of time with intervals of 20 seconds using a spectrophotometer for 10 minutes. These “absorbance vs time” plots were then used to obtain the slope at the initial point (Slope_{Initinal}) of each reaction (over first 150 seconds).

The initial reaction velocity (v) was calculated by Slope_{Initial}/(ε₆₅₂ₙₘ×l), where ε_{TMB-652 nm} is the molar extinction coefficient of TMB at 652 nm, which is 3.9×10⁴ M⁻¹ cm⁻¹. The plots of reaction velocity, v, against TMB and H₂O₂ concentrations were fitted using nonlinear regression of the Michaelis-Menten equation. The kinetic parameters were calculated based on the Michaelis-Menten equation: v=Vmax×[S]/(Km+[5]), where

Vmax represents the maximal reaction velocity, [5] is the concentration of substrate, and Km is the Michaelis constant. Vmax was obtained from fitting to the model using GraphPad Prism software, and the catalytic constant (Kcat) was calculated using the equation: Kcat=Vmax/[E], where [E] is the AuNC concentration.

### AuNC Complex Assembly

In a typical conjugation, 125 μL NeutrAvidin Protein (120 μM, PBS, Thermo Fisher, NAv) was mixed with 1 mL of AuNC-P1 or AuNC-P2 (20 μM) and incubated for 12 hours gently shaking (500 rpm) at 37° C. Unbound AuNCs were removed from AuNC-NAv complexes through centrifugal ultrafiltration (Amicon Ultra centrifugal filter units Ultra-15, MWCO 50 kDa, Sigma), where AuNC-NAv complexes remained in concentrate and any unbound AuNCs were collected in the filtrate. After ultrafiltration, AuNC-NAv complexes were resuspended in PBS (30 μM by [AuNC]) and sterile filtered (Millex-GV Filter, Millipore, 0.22 μm).

### In Vitro Gel Filtration Chromatography Assays

AuNC-NAv complexes were first incubated with a recombinant enzyme: MMP9 (Active, Human, Recombinant, PF140, Merk Millipore); MMPI (Active, Human, Recombinant, E. coli, 444270, Merk Millipore); MMP13 (Active, Human, Recombinant, 444287); or thrombin from human plasma (T7009, Sigma, 100 units/mL in a 0.1% (w/v) bovine serum albumin solution).

Enzyme and AuNC-NAv were incubated at 37° C. gently shaking (500 rpm). Incubation times varied (1-12 hours) and concentration of enzyme varied (50-100 nM), where the final peptide substrate concentration was maintained at >1000 molar excess to enzyme concentration (see the brief descriptions of the figures).

Three identical glass chromatography columns were packed with Sephacryl 5200 high resolution resin (column D: 1 cm, H: 18 cm, resin: GE Healthcare Life Sciences, fractionation range for globular proteins 5-250 kDa) to separate samples based on size. Columns were thoroughly cleaned between experiments with PBS. In a typical GFC experiment, ca. 200 μL of 10 μM AuNC-PX, AuNC-PX-NAv, and AuNC-PX-NAv+50 nM enzyme (after incubation) were loaded onto each column in parallel. As soon as the sample was added to the resin bed, 24, 500 μL fractions were collected into individual Eppendorf tubes, while PBS was added to the column reservoir. After fractions were collected, a catalytic activity assay was performed on the samples. For the activity assay, 100 μL of each fraction was added to a 96-well plate, followed by 100 μL substrate solution (1-Step Ultra TMB ELISA Substrate Solution with 4 M H₂O₂). The absorbance of the reaction solution at 652 nm was monitored up to 25 minutes after addition of substrate, corresponding to oxidation of TMB by H₂O₂. The composition of the sample could be determined based on how quickly it eluted from the column as measured by activity. Larger AuNC complexes elute within the first 7 mL, and smaller bare AuNCs elute more slowly and are found in 7-12 mL, corroborated by DLS of column fractions. Absorbance at a fixed time point was plotted as a function of eluted volume, where clear peaks in absorbance are associated with either AuNC-NAv complexes or bare AuNCs. For AuNC-NAv complexes incubated with enzymes, the proportion of liberated AuNCs could be measured by calculating the area under the curve corresponding to 7-12 mL eluted volume (fractions corresponding to bare AuNCs).

### Fluorescence Correlation Spectroscopy

For FCS analysis, AuNC-P1 and AuNC-P2 were labelled with 50 molar excess reactive dye (Oregon Green 488 Carboxylic Acid, Succinimidyl Ester, 6-isomer, Thermo Fisher), further labelled AuNC-PX-OG. Unreacted dye was removed using Zeba Spin Desalting Columns 7K MWCO (Thermo Fisher). AuNC-PX-OG-NAv complexes were assembled following above protocol and purified to remove unbound AuNC-PX-OG. AuNC-PX-OG-NAv complexes were further incubated with enzymes, and kinetics of AuNC complex disassembly via substrate cleavage was monitored over time using FCS.

Sample Preparation for Measuring Enzyme Cleavage Kinetics For MMP9: 0.33 μL MMP9 stock (Merck PF140 lot#2872521, 0.1 mg·mL⁻¹˜1500 nM, 57.28 Units/h/μg P) was added per 10 μL ample stock (20 μM, AuNC), for a final enzyme concentration of 50 nM, with AuNCs in 400 molar excess to MMP9. Since AuNCs bear ca. 20 peptide substrates per particle, there was ca. 8000 molar excess peptide substrates per enzyme. Estimate based on MMP rate/peptide concentrations how long it would take to cleave peptide substrates.

For thrombin: 0.58 μL thrombin stock (100 U/ml, 32 μg/ml˜860 nM) was added per 10 μL sample stock (20 μM, AuNC), for a final enzyme concentration of 50 nM, with AuNCs in 400 molar excess to thrombin.

All enzyme incubations were performed at 37° C., and incubations longer than 3 hours were maintained at 37° C. while shaking (300 rpm). Samples were then diluted in PBS for FCS measurements.

### FCS Measurements

FCS was performed on a commercial LSM 880 (Carl Zeiss, Jena, Germany) equipped with an incubation chamber. All measurements were performed at 37° C. An Ar⁺ laser was used as excitation source for the 488 nm wavelength. Appropriate filter sets were used to detect the fluorescence signal (LP 505). The laser beam passed through a 40× C-Apochromat water immersion objective with a numeric aperture of 1.2 to focus the beam into the sample droplet. Measurements were performed 200 μm above the ibidi 8-well bottom plate (80826, ibidi, Germany) using a 5 μL droplet of sample for each condition. OregonGreen 488 carboxylic acid in PBS (OG488, 06149, ThermoFisher Scientific, NHS-ester was first deactivated by overnight incubation in PBS at room temperature) was used as a standard to calibrate the beam waist (D=4.1×10⁻⁶ cm²/s at 25° C., and when corrected for the higher temperature used: D=5.49×10⁻⁶ cm²/s at 37° C.)(Kapusta, PicoQuant GmbH Appl. Note 2010). Immediately before the measurement, stocks or incubated samples were diluted 100-fold in pre-warmed PBS and 5 μL was placed into the measuring chamber. The sample was equilibrated and bleached for 5×5 seconds and 25×5 seconds, intensity traces were recorded, autocorrelated and analyzed for each sample. Autocorrelation curves were created in ZEN software (Carl Zeiss, Jena, Germany) and the curves were exported for further analysis using PyCorrfit program 1.1.1(Müller et al., Bioinformatics. 2014, 30, 2532-2533). For all the graphs, data for the 25 curves are given except for the autocorrelation curves, which are always the average curve for the whole measurement (125 s). First, stocks of clusters/complexes were fitted using one component fits (G_{1comp}(τ)) to obtain the diffusion times for the pure components. Second, samples incubated with enzymes were fitted with two component fits (G_{2comp}(τ) with one component fixed to pure cluster diffusion (τ₁) and the other fixed to pure complex diffusion (τ₂) to yield the fraction of free clusters (F₁), which is equivalent to the fraction cleaved. A triplet fraction with a triplet time of 10 μs was included for all the curves.

The following equation relates the x-y dimension of the confocal volume (ω²_{xy}), which was calibrated by a standard measurement of OG488 in PBS, to the diffusion coefficient (D), which was calculated for each sample using the obtained diffusion time (τ_{D}):

$$D = \frac{{ω}_{xy}^{2}}{4 {τ}_{D}}$$

Stokes-Einstein equation was used to calculate hydrodynamic radii (Rₕ) via the obtained diffusion coefficients.

In Vitro Cleavage Assays with Quenched Substrates

Q1 (1 uM by peptide) was incubated with recombinant mouse thrombin (12.5 nM working concentration; Haematologic Technologies) in a 384-well plate at 37° C. in PBS-BSA (0.1% w/v). Q2 (1 uM by peptide) was incubated with recombinant human MMP-9 (100 nM working concentration; Enzo Life Sciences) in activity buffer (50 mM Tris, 150 mM NaCl, 5 mM CaCl₂, 1 uM ZnCl₂) containing 0.1% BSA. Fluorescence dequenching was monitored at 37° C. using a Tecan Infinite microplate reader.

### In Vivo Renal Clearance Studies.

All animal studies were approved by the Massachusetts Institute of Technology (MIT) committee on animal care (MIT protocol 0417-025-20). GSH-templated and substrate functionalized AuNCs were diluted to 10 uM [AuNC] in sterile PBS. Wild-type female Swiss Webster mice (4-6 weeks, Taconic) were intravenously administered 2000 pmol AuNCs via the tail vein. After nanocluster injection, mice were placed in custom housing with a 96-well plate base for urine collection. After 1 hour, their bladders were voided, and collected urine volume was measured. Clearance of active AuNCs was quantified via catalytic activity assay, and urine gold content was quantified by ICP-MS.

### Urine Catalytic Activity Assays

For all assays, 25 uL of urine was diluted into 25 uL PBS in a transparent 96-well plate and allowed to equilibrate at room temperature for 15 minutes. 100 uL each of 5M H₂O₂ (Sigma) and TMB (ThermoFisher Scientific) were then added, and the plate was read kinetically at 652 nm over the course of 30 minutes. For renal clearance studies, the concentration of active AuNCs present in the urine was quantified via reference to a ladder of known AuNC concentrations. For disease detection studies, the initial reaction velocity was quantified as the rate of change of the absorbance over the first 10 minutes of the reaction.

### ICP-MS on Urine Samples

Urine samples were digested using aqua regia for 24 h. The digested samples were further diluted in an ICP-MS matrix composed of 4% HCl/4% HNO₃. The gold content in each sample was measured using an Agilent 7900 ICP-MS using an indium internal standard (5 ppb) and gold standard (TraceCERT, Sigma) for the calibration curve prepared in the ICP-MS matrix.

### Cell Culture

For xenograft studies, LS174T (ATCC) cells were cultured in Eagle's Minimal Essential Medium (EMEM, ATCC) supplemented with 10% FBS (Gibco) and 1% penicillin-streptomycin (CellGro). For in vtiro cytotoxicity assays, HEK293T (ATCC) cells were cultured in Dulbecco's Modified Eagle Medium (DMEM, ATCC) supplemented with 10% FBS (Gibco) and 1% penicillin-streptomycin (CellGro). Cells were passaged when confluence reached 80%.

In vitro cytotoxicity studies. For in vitro cytotoxicity studies, HEK293T cells were plated in a 96-well plate (10,000 cells per well) and allowed to adhere to the wells. 24 h post seeding, cells were incubated with varying concentrations of AuNC-avidin complex (diluted in PBS) for 24 h. Cell viability was evaluated using the MTS (3-(4,5-dimethylthiazol-2-yl)-5-(3-carboxymethoxyphenyl)-2-(4-sulfophenyl)-2H-tetrazolium) assay (Promega).

Pharmacokinetic studies. To analyze blood half-life of the avidin carrier, female Swiss Webster mice (4-6 weeks, Taconic) were injected with neutravidin-biotin labeled with the near-infrared dye VivoTag750 (VT750, PerkinElmer; 1 uM by VT750). Blood was withdrawn retro-orbitally (˜70 uL) and then immediately transferred into 40 uL of PBS with 5 mM EDTA and centrifuged to pellet blood cells. Concentration of avidin carrier was measured using an Odyssey infrared scanner (Li-Cor Inc.). For biodistribution studies, nude mice bearing LS174T flank tumors were infused with labeled avidin-biotin. Mice were sacrificed 1 hour post injection, and organ and tumor accumulation was measured using an Odyssey scanner and quantified using ImageJ (NIH).

In vivo cytotoxicity studies. AuNC-avidin complex (AuNC-P1₂o-NAv, 3000 pmol) was intravenously injected into immunocompetent female Swiss Webster mice (4-6 weeks, Taconic). The mass of each mouse was monitored for 4 weeks post injection and compared with control mice. Kidney, liver, and spleen tissues were collected from the mice 4 weeks after injection, fixed in 10% formalin, paraffin embedded, stained with haematoxylin and eosin, and then examined by a pathologist.

Colorectal cancer xenograft studies. Female NCr Nude mice (4-5 weeks, Taconic) were injected bilaterally with 3×10⁶ LS174T cells per flank. Two weeks after inoculation, tumor-bearing mice and age-matched controls were injected with 15 uM MMP-sensitive AuNC nanosensors in 200 uL of PBS (concentrations determined by AuNC). After nanosensor injection, mice were placed in custom housing with a 96-well plate base for urine collection. After 1 hour, their bladders were voided to collect between 100-200 uL of urine. Urine was analyzed via the catalytic activity and ICP-MS measurements described above. AuNC-PEG-NAv nanoparticles were injected at 10 uM in 200 uL PBS in an independent cohort of mice, with analysis proceeding similarly.

Statistical analyses. All statistical analyses were conducted in GraphPad 7.0 (Prism).

### Example 8

### Renal Clearable Catalytic Gold Nanoclusters for In Vivo Disease Monitoring. Peptide-Templated Catalytic AuNCs with High Serum Stability

Protease-responsive nanosensors were synthesized using biotinylated protease-cleavable peptides to template and stabilize the growth of catalytic AuNCs, which were further coupled to neutravidin (NAv). Neutravidin was selected as a biocompatible carrier for protease-responsive AuNC reporter probes due to its high affinity for biotin and low nonspecific binding properties (FIGS. 1A-1C) (Jain et al., Mol. Pharm. 14, 1517-1527 (2017)) The AuNC-neutravidin (AuNC-NAv) complex was then intravenously (i.v.) administered and specifically disassembled by proteases at the site of disease. The system takes advantage of a biological pharmacokinetic switch, where the size of the particle largely drives biodistribution (Soo Choi et al., Nat. Biotechnol. 25, 1165-1170 (2007); Du et al., Nat. Rev. Mater. 3, 358-374 (2018)). Once proteolytically liberated from the neutravidin complex, AuNCs circulated via the bloodstream and were efficiently filtered into the urine through the kidneys due to their small size (<5 nm). A simple colorimetric assay was performed on the urine to assess the presence of AuNCs as an indicator of disease state (FIGS. 1A-1C). A co-templated approach was used to synthesize noble metal nanoclusters, incorporating both the tripeptide glutathione (GSH, γ-Glu-Cys-Gly), a common capping ligand in nanocluster synthesis, (Zhang et al., Sci. Rep. 5, 8669 (2015); Luo et al., J. Am. Chem. Soc. 134, 16662-16670 (2012))and a thiol-terminated functional protease-cleavable peptide (Table 2) that act as both stabilizing capping ligands and reducing agents for nanoparticle formation (FIG. 2A). Gold was selected as the core metal, as it exhibited the highest catalytic activity compared to platinum and gold-platinum bimetallic hybrid nanoclusters and could be produced with a low coefficient of variation (CoV=8.5%), an important consideration in designing a scalable diagnostic platform (FIGS. 7A-7B and FIGS. 7E-7F).

The peptide substrates used as templates for AuNC synthesis were composed of three functional domains: an enzyme recognition motif, a C-terminal cysteine residue to provide a thiol group for sequestering Au ions, and an N-terminal biotin ligand for efficient conjugation to a neutravidin carrier protein. The advantage of this synthesis route to produce catalytic noble metal nanoclusters is the ability to incorporate responsive and functional ligands onto the surface through simple gold-thiol interactions in a one-pot synthesis. It was determined whether the target protease may be sterically hindered from accessing the scissile bond when the peptide sequence is presented on the AuNC and simultaneously linked to the neutravidin core. To explore this hypothesis, longer peptides (P1₂₀, P2₂₀) were also synthesized by incorporating glycine spacers between the N-terminus and protease recognition motif (Table 3). The ability of the relevant protease to cleave the peptide substrate was assessed by verifying the mass of fragments after in vitro protease degradation (FIGS. 6A-6F).

The AuNCs did not exhibit surface plasmon resonance, a characteristic of large gold nanoparticles, but rather exhibited molecular-like absorption and corresponding fluorescence properties, attributed to the discrete electronic state arising from their size (FIGS. 7C-7D). Transmission electron microscope (TEM) images and size analysis of the peptide-templated AuNCs (FIG. 2B, FIGS. 8A-8H and FIGS. 9A-9D) showed that the average size (1.5±0.4 nm, FIG. 2C) was below the glomerular filtration cut-off (ca. 5.5 nm), making them ideally suited for kidney clearance.(Soo Choi et al., Nat. Biotechnol. 25, 1165-1170 (2007); Du et al., Nat. Rev. Mater. 3, 358-374 (2018); Yu et al., Angew. Chemie—Int. Ed. 55, 2787-2791 (2016); Ning et al., APL Mater. 5, (2017); Liu et al., J. Am. Chem. Soc. 135, 4978-4981 (2013)).

The peroxidase-like catalytic activity of the AuNCs was measured using the oxidation of the peroxidase substrate 3,3′,5,5′-Tetramethylbenzidine (TMB) by H₂O₂ as a model catalytic reaction, and absorbance at 652 nm provided a colorimetric readout of AuNC activity (FIG. 2D, FIGS. 7A-7B, FIGS. 7E-7F, FIGS. 10A-10D, and FIG. 10F, Table 7). To assess the sensitivity of the catalytic reporter probes, the catalytic activity of a dilution series of each AuNC batch in synthetic urine was measured (FIG. 2E) and determined the limit of detection to be ca. 2.7 picomoles (25 μL, urine, ca. 100 nM AuNCs), with a broad linear response and dynamic range spanning over three orders of magnitude of particle concentration.

In Table 7, steady-state kinetic assays were carried out at room temperature in a 96-well plate with 220 μL solution with estimated path length (l) of 0.5 cm. 25 mM NaOAc/HOAc solution (pH 4.0) was used as the reaction buffer. For kinetic assays varying TMB concentration, AuNCs (20 μM, 20 μL) were mixed with TMB (10 μM to 1 mM, 100 μL) and H₂O₂ (5 M, 100 μL). The initial reaction velocity (v) was calculated by Slope_{Initial}(ε_{TMB-652 nm}×l), where ε_{TMB-652 nm} is the molar extinction coefficient of TMB at 652 nm, which is 3.9×10⁴ M⁻¹-cm⁻¹. The plots of reaction velocity, v, against TMB concentrations were fitted using nonlinear regression of the Michaelis-Menten equation. The kinetic parameters were calculated based on the Michaelis-Menten equation: v=Vₘₐₓ×[S]/(Kₘ+[S]), where Vₘₐₓ represents the maximal reaction velocity, [S] is the concentration of substrate, and Kₘ is the Michaelis constant. Vₘₐₓ was obtained from fitting to the model using GraphPad Prism software.

There are several advantages to using inorganic AuNCs over natural peroxidases.

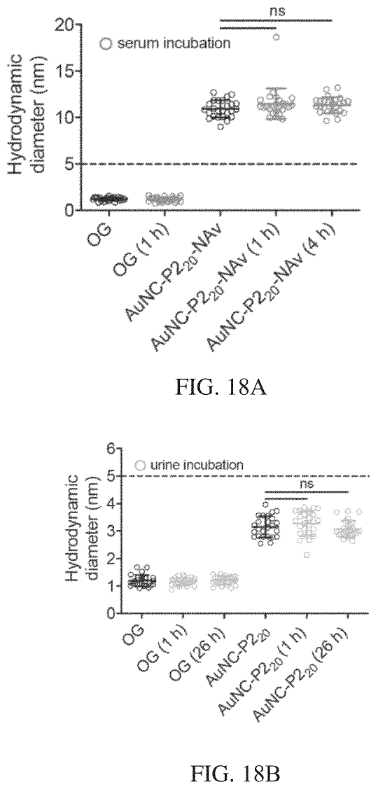

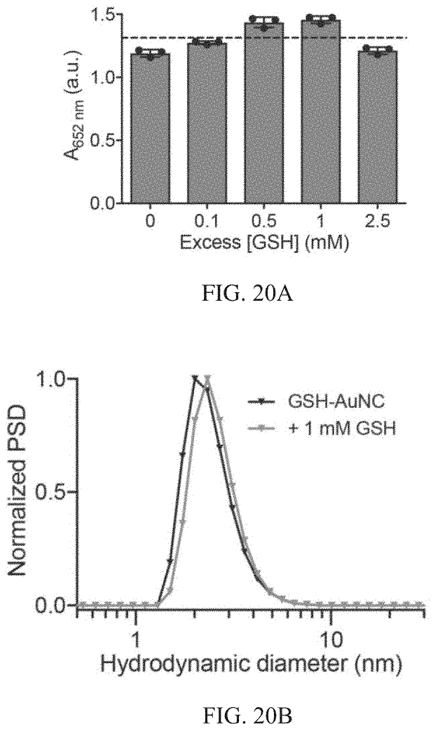

HRP is not feasible to use as a reporter probe in a comparable in vivo diagnostic system, as it is not readily cleared through the renal filtration pathway due to its size (ca. 4.5 nm) and the tendency for proteins to be reabsorbed by the tubular epithelium (Straus Kidney Int. 16, 404-408 (1979)). Additionally, HRP would be susceptible to nonspecific degradation by endogenous proteases in vivo which would hinder activity of any cleared enzyme (Manning et al., Pharm. Res. 27, 544-575 (2010)). On the other hand, AuNCs showed high stability in physiological environments, maintaining catalytic activity, size, and morphology in the presence of serum, urine, and physiologically relevant glutathione concentrations (FIG. 2F, FIGS. 18A-18B, FIGS. 19A-19D, and FIGS. 20A-20B). A key performance requirement of the AuNCs is that they retain their catalytic activity following exposure to complex environments such as patient serum, which contains ca. 7 wt % protein. AuNCs effectively evaded nonspecific protein adsorption, retaining 80-90% of catalytic activity after 1 h incubation in fetal bovine serum (undiluted FBS) or synthetic urine compared to PBS controls (FIG. 2F). In deciding which particle platform to take forward in vivo, a system was selected that balanced appropriate protease substrate loading with retention of activity (FIGS. 11A-11D).

TABLE 7
Comparison of the kinetic parameters of catalysts toward the
oxidation of TMB by H₂O₂.

| Catalyst | [E]/M | Substrate | Kₘ/M | Vₘₐₓ/M s⁻¹ | K_{cat}/s⁻¹ |
|---|---|---|---|---|---|
| AuNC ca. | 1.8 × 10⁻⁶ | TMB | 2.3 × 10⁻⁴ | 3.6 × 10⁻⁷ | 0.20 |
| 1.5 nm | | | | | |
| HRP* | 2.5 × 10⁻¹¹ | TMB | 4.3 × 10⁻⁴ | 1.0 × 10⁻⁷ | 4.0 × 10³ |

[E] represents the catalyst concentration, Kₘ is the Michaelis constant, Vₘₐₓ is the maximal reaction velocity, and K_{cat} is the catalytic constant that equals Vₘₐₓ/[E].
*Gao, L. et al. Nat. Nanotechnol. 2, 577-83 (2007).

### Renal Clearance of AuNCs and Activity Retention in Urine

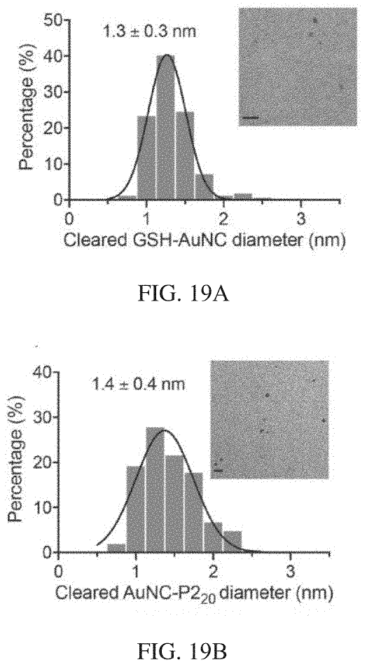

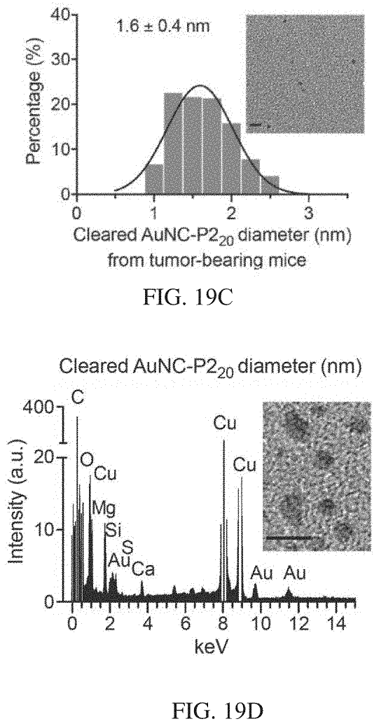

The high physiological stability and retention of AuNC catalytic activity after exposure to serum and urine offered a unique opportunity to non-invasively monitor AuNC clearance in urine by measuring gold signal using both the catalytic activity assay and inductively coupled plasma-mass spectrometry (ICP-MS) (FIG.3A). To determine renal clearance efficiency, urine from mice injected with AuNCs was measured against a calibration curve for both catalytic activity and gold content. This showed that up to 73±7% of the injected dose of functionalized AuNCs left the body via this route at 1 h post injection (p.i.) and retained catalytic activity in urine (FIG. 3B). Encouragingly, the catalytic activity assay and ICP-MS results appeared to correlate (FIG. 3C, Pearson's r=0.492, *P=0.0383). Thus, the catalytic activity assay can provide a simple and sensitive assessment of AuNC presence in urine without the need for ICP-MS. Analysis of urine from mice injected with PBS revealed that no endogenous peroxidase activity was detectable in collected urine (FIGS. 12A-12B). Using TEM image analysis, it was confirmed that the size and morphology of AuNCs cleared by the kidneys and excreted into the urine was comparable to as-synthesized AuNCs (FIGS. 19A-19D). This indicates that the particle stability was unperturbed in vivo, which is consistent with the retention of the functional properties of the nanoclusters after in vivo interrogation.

### AuNC Nanosensors Respond io Protease Activity In Vitro

The biotin functional handles on the protease substrate-modified AuNCs were used to tether them to a neutravidin carrier protein to assemble an AuNC-NAv complex. Dynamic light scattering (DLS) was used to monitor the size of the free AuNCs, neutravidin carrier, and assembled AuNC-NAv complex (FIGS. 13A-13F and FIGS. 14A-14F), with representative hydrodynamic diameters of 2.5±0.6 nm (GSH-AuNC), 3.3±0.7 nm (AuNC-P1₂₀), 7.9±1.5 nm (NAv), and 11.3±2.2 nm (AuNC-P1₂₀-NAv).

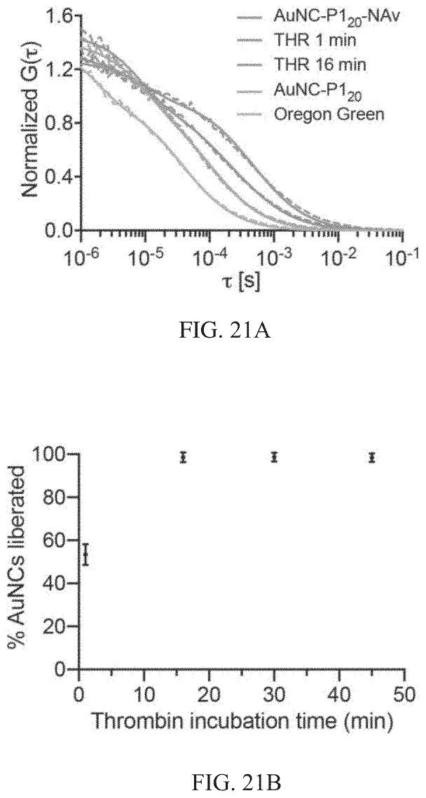

To explore the kinetics of proteolytic cleavage of AuNC-NAv complexes, fluorescence correlation spectroscopy (FCS) was employed as a single-molecule detection method (FIG. 4A). After enzyme incubation, the diffusivity of the complex shifted over time towards that of the free fluorescently labelled clusters, indicating cleavage had occurred (FIG. 4B). Hydrodynamic size analysis by FCS showed that the MMP-responsive AuNC-P2₂₀-NAv complex was completely disassembled within 4.5 h of MMP9 incubation (FIG.4C). The size of the thrombin-cleavable complex, AuNC-P1₂₀-NAv, did not significantly change when incubated with MMP9, and the size of the MMP-responsive AuNC-P2₂₀-NAv complex did not fall below the renal filtration limit when incubated with an off-target enzyme, in this case thrombin, for 12 h. Taken together, these results show the specificity of the nanosensors for their target enzymes. To demonstrate the modularity of the system, FCS was used to measure the disassembly kinetics of the thrombin-responsive complex (AuNC-P1₂₀-NAv), which was efficiently cleaved by thrombin (FIGS. 21A-21B). Further, MMP9 exhibited a rate of 3% AuNCs cleaved per minute toward the AuNC-P2₂₀-NAv complex, while the rate was only 0.08% AuNCs cleaved per minute toward the AuNC-P2₁₃-NAv complex (FIG. 4D). This ca. 40-fold increase in the cleavage rate for the complex formed with the longer linker could be attributed to increased accessibility of the enzyme to the scissile bond. FCS results showed that in the presence of biologically-relevant enzyme concentrations (Kwong et al., Proc. Natl. Acad. Sci. 112, 12627-12632 (2015)) significant cleavage was observed for AuNC-P2₂₀-NAv complexes, where 80% of AuNCs were cleaved within the first hour of incubation with MMP9.

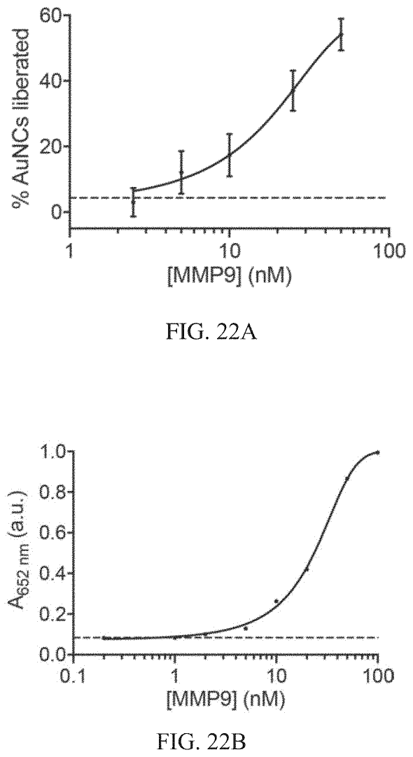

Proteolytic cleavage of AuNC-NAv complexes was further characterized in vitro by incubating complexes with recombinant protease, using gel filtration chromatography (GFC) to separate cleavage products by size, and monitoring cleavage with a catalytic activity assay (FIGs.4E-4F and FIGS. 14A-14F). The extent of cleavage of the AuNC-NAv complex under different conditions was quantified by analysing the area under the curve associated with each cleavage product from the activity assay (Table 8). Nonspecific cleavage was investigated by incubating AuNC-P1₂₀-NAv with MMP9 and AuNC-P2₂₀-NAv with thrombin. Low background cleavage by the off-target enzyme was observed (FIGS. 4E-4F), in agreement with FCS results. Finally, the sensitivity of the nanosensor to MMP9 activity was determined in vitro using both FCS and a filtration-based colorimetric catalytic activity assay (FIGS. 22A-22B), where low nanomolar sensitivities were observed, comparable to commercial in vitro fluorogenic protease activity assays.

TABLE 8
Quantification of AuNC cleavage products from in vitro gel filtration
chromatography assays.

| | % Cleavage (free AuNC | Figure |
|---|---|---|
| AuNC-NAv complex | fraction: 7-12 mL) | reference |
| AuNC-P1₁₃-NAv + THR (12 h) | 21.8% | FIG. 14F |
| AuNC-P1₂₀-NAv + THR (12 h) | 90.2% | FIG. 4E |
| AuNC-P1₂₀-NAv + MMP9 (12 h) | 7.1% | |
| AuNC-P2₁₃-NAv + MMP9 (12 h) | 15.2% | FIG. 14D |
| AuNC-P2₂₀-NAv + MMP9 (12 h) | 75.1% | FIG. 4F |
| AuNC-P2₂₀-NAv +THR (12 h) | 5.5% | |
| AuNC-P2₂₀-NAv + MMP7/9/13 | MMP7: 12.1% | FIG. 14E |
| (12 h) | MMP9: 54.9% | |
| | MMP13: 29.8% | |
| AuNC-1:20-P1₂₀-NAv + THR (1 h) | 89.9% | FIG. 14F |
| AuNC-1:5-P1₂₀-NAv + THR (1 h) | 49.6% | |

### Biodistribution and Clearance Pathways for Nanosensors

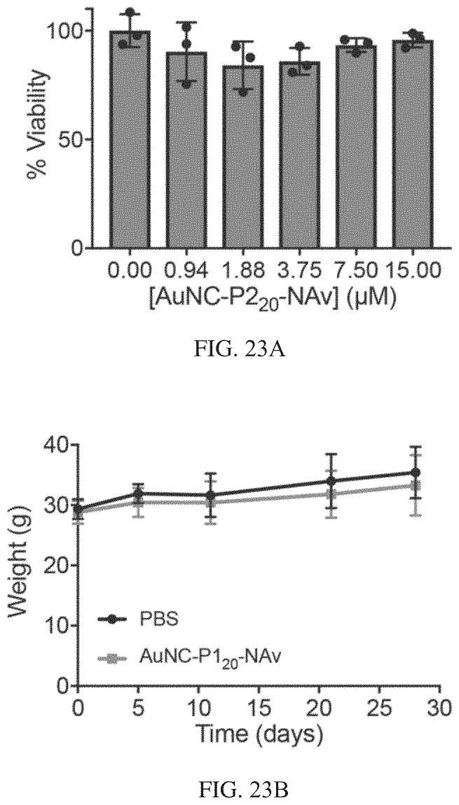

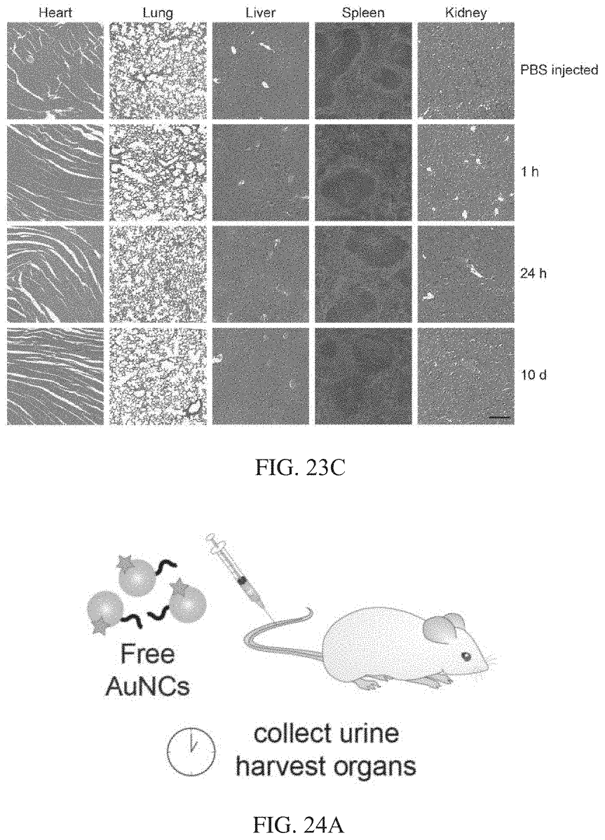

The biocompatibility of AuNC-NAv complexes was assessed in vitro and found that they were non-toxic to HEK293T cells up to 15 μM (FIGS. 23A-23C). Toxicological responses of the AuNC-NAv complexes (3000 pmol AuNC dose) in vivo was investigated by examining pathology of the mice after complex injection. No significant changes in bodyweight over 28 days and no histological evidence of heart, lung, liver, spleen, or kidney toxicity were found at both short (1 h) and longer (24 h and 10 days) time points post injection, suggesting that AuNC-NAv complexes did not induce significant systemic toxicity (FIGS. 23A-23C).

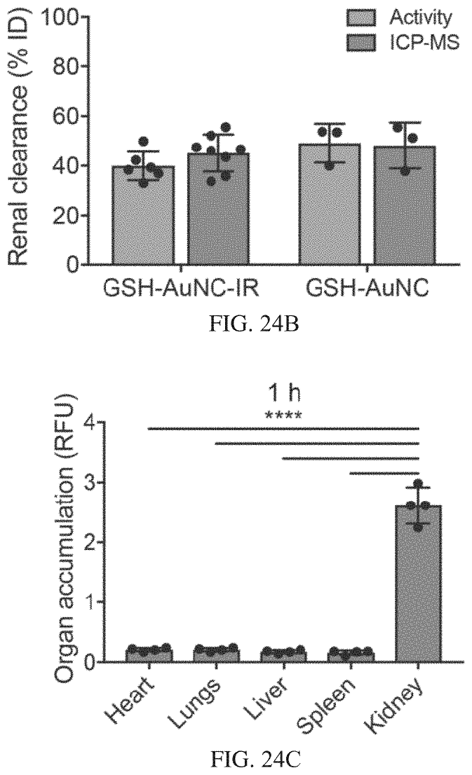

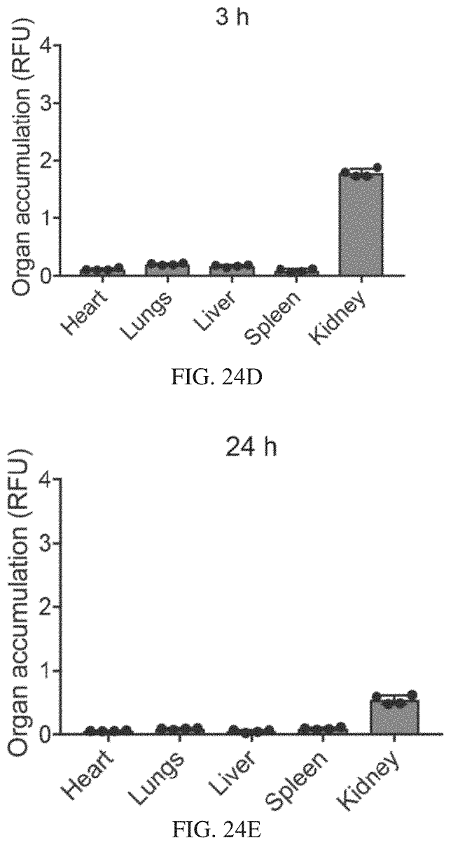

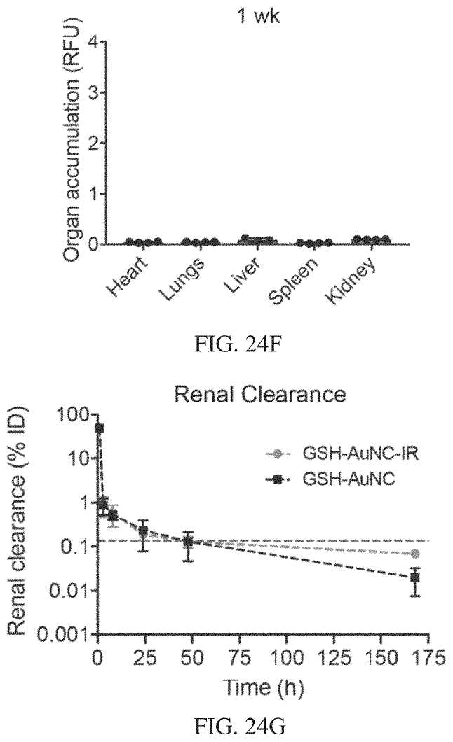

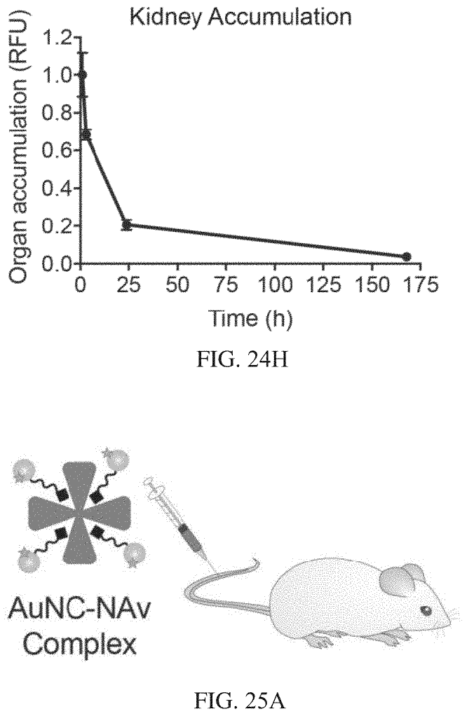

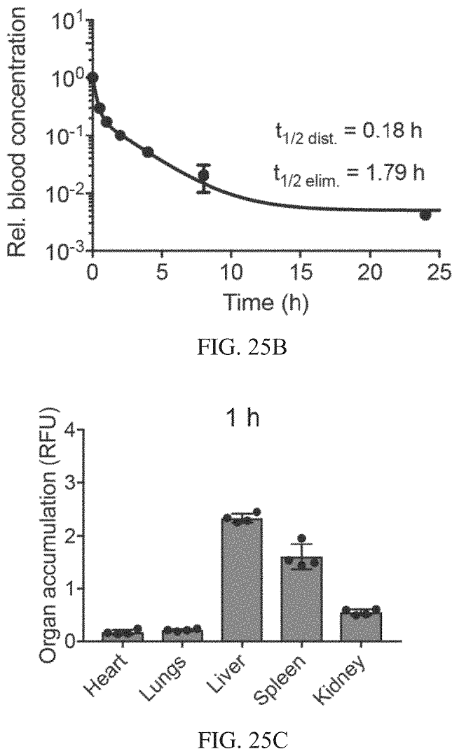

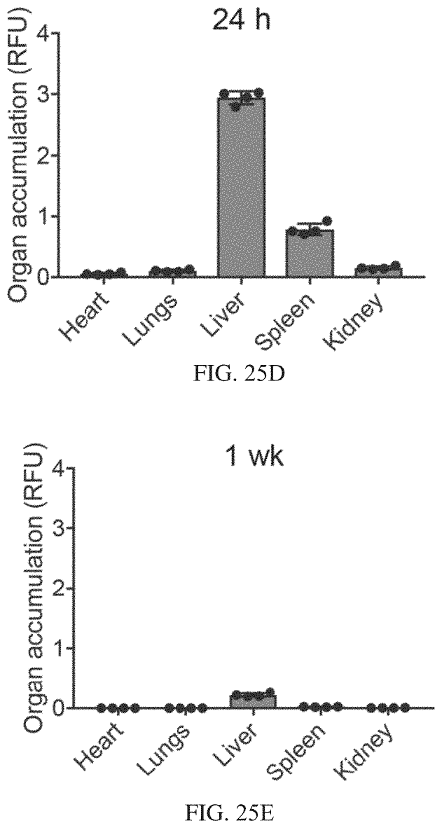

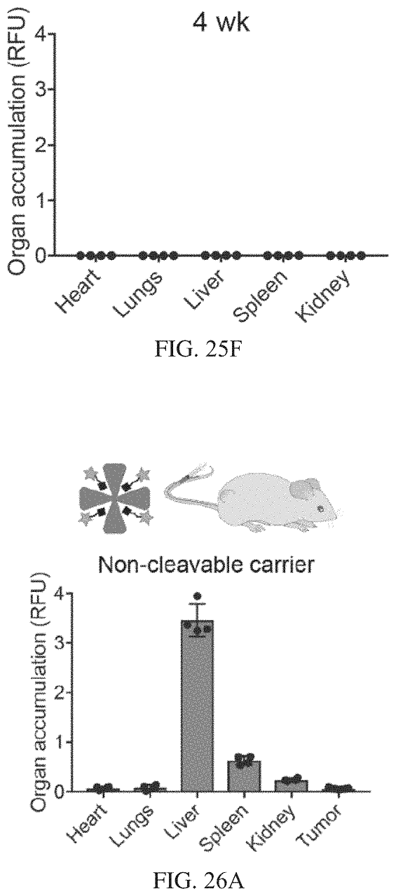

To assess clearance time frames and mechanisms, the organ biodistribution, blood pharmacokinetics, urine composition, and elimination pathways of AuNCs and AuNC-NAv complexes labelled with a photostable near-IR dye were determined. From the organ biodistribution study, free AuNCs accumulated most significantly in the kidneys relative to other organs including the liver at 1 h p.i. and were completely cleared from all major organs within 7 days p.i. To corroborate the biodistribution study, gold signal in the urine was measured by ICP-MS and the catalytic activity assay, where the presence of AuNCs was undetectable after 24 h p.i. (FIGS. 24A-24H, Table 9). In Table 9, urine samples were collected at varying time points from mice injected with either GSH-AuNC-IR or unlabelled GSH-AuNCs (mean±s.d., n=4 mice per group), where comparable renal clearance efficiencies were observed between bare and IR dye-labelled AuNCs. Gold signal was undetectable in urine after 24 h p.i., where limit of detection was calculated as 3 standard deviations above the mean gold signal from PBS injected control mice (cut-off=0.13% ID). Due to their size (ca. 11 nm), the intact AuNC-NAv complexes accumulated predominately in reticuloendothelial system (RES) organs (Yu et al., ACS Nano 9, 6655-6674 (2015)). The AuNC-NAv signal in the liver increased up to 24 h p.i., significantly decreased after 1 week, and was completely undetectable in all major organs 4 weeks p.i. (FIGS. 25A-25F). Encouragingly, the biodistribution and histology results suggest that, in healthy animals, intact AuNC-NAv complexes were cleared from the circulation and taken up in RES organs and eliminated completely through hepatic (bile to faeces) and renal (urine) excretion within 4 weeks p.i. with no evidence of systemic or tissue-level toxicity.

TABLE 9
Gold content analysis in urine as measured by ICP-MS.

| | GSH-AuNC-IR | GSH-AuNC |
|---|---|---|
| | (% ID by ICP- | (% ID by ICP- |
| Time (h) | MS) | MS) |
| 0-1 | 45.13 ± 7.46 | 48.20 ± 9.23 |
| 2-3 | 0.89 ± 0.43 | 0.87 ± 0.36 |
| 7-8 | 0.57 ± 0.30 | 0.52 ± 0.13 |
| 23-24 | 0.19 ± 0.05 | 0.23 ± 0.15 |
| 47-48 | 0.13 ± 0.03 | 0.13 ± 0.08 |
| 167-168 | 0.07 ± 0.01 | 0.02 ± 0.01 |
| 407-408 | — | 0.02 ± 0.01 |

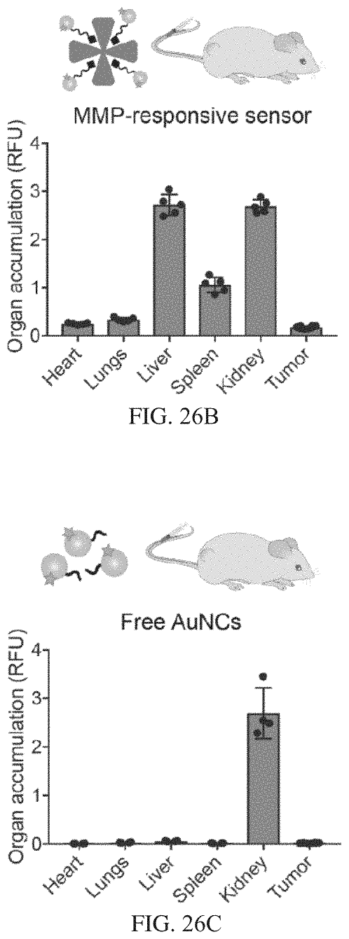

AuNC nanosensors enable colorimetric urinary disease detection After confirming successful cleavage by recombinant proteases in vitro, it was sought to apply the protease-responsive AuNC nanosensor platform to in vivo disease detection using the colorimetric urinary readout. The pharmacokinetics of the neutravidin carrier, AuNC-NAv complex, and free AuNCs was characterized in terms of accumulation in organs and tumour xenografts of the human colorectal cancer cell line LS174T, which secretes MMP9 (Warren et al., Proc. Natl. Acad. Sci. U. S. A. 111, 3671-6 (2014) (FIGs. FIGS. 26A-26C). Based on the measured blood half-life of the AuNC-NAv complex and the degree of tumour accumulation within 1 h p.i., 1 h after nanosensor injection was selected as the time point for urine collection.

For in vivo tumour detection experiments, tumour-bearing and healthy control mice were intravenously injected with MMP-responsive AuNC-P2₂₀-NAv nanosensors (FIG. 5A).

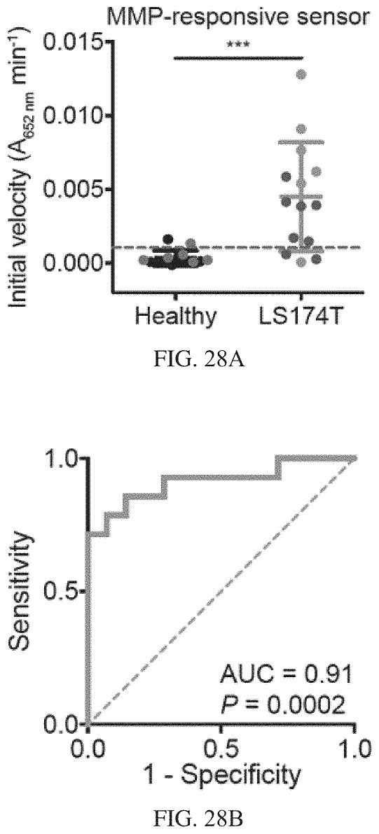

Urine was collected from mice 1 h p.i., and the catalytic activity assay was run using 25 μL of urine sample. Comparing signal from healthy and tumour-bearing mice, a blue colour was observed that could be read by eye in urine samples from tumour-bearing mice after the addition of the chromogenic peroxidase substrate, TMB (FIG. 5B). Quantification revealed a mean urinary signal increase of approximately 13-fold in tumour-bearing mice relative to healthy mice, as measured by the direct colorimetric readout and initial velocity analysis (A652 min⁻¹) of cleared AuNC catalytic activity in collected urine (FIG. 28A). The AuNC catalytic activity measured corresponded to ca. 3.2% of the injected dose in urine from tumour-bearing mice compared to 0.2% renal clearance in healthy mice, normalized using urine volumes (FIGS. 16A and 16C). Without being bound by a particular theory, the platform disclosed herein might benefit from improved diffusion, transport, tumour accumulation, and clearance properties of peptide-templated gold nanoclusters compared to larger nanomaterials commonly used in delivery applications, where only ca. 0.7% of the administered nanoparticle dose was reported to be delivered to the solid tumour (Dai et al., ACS Nano 12, 8423-8435 (2018); Tang et al., Angew. Chemie—Int. Ed. 55, 16039-16043 (2016); Wilhelm et al., Nat. Rev. Mater. 1, 16014 (2016)). Receiver operating characteristic

(ROC) analysis revealed that the colorimetric test was highly accurate and discriminated the presence of colorectal cancer xenografts with an area under the curve (AUC) of 0.91 (FIG. 28B, P=0.0002). Furthermore, the delivery of the nanosensors to malignant tissues can be enhanced by exploiting the one-pot synthesis scheme for the incorporation of active targeting ligands, e.g., the integrin-targeting ligand iRGD (Kwon et al., Nat. Biomed. Eng. 1, 0054 (2017)) onto the surface of the AuNCs.

Having established that the MMP-responsive AuNC nanosensors could discriminate between tumour-bearing and healthy mice, it was determined whether the urinary signal was driven by disease-associated protease activity. There was no significant difference in urine volumes between the groups, and analysis of urine samples from PBS-injected healthy and tumour-bearing mice confirmed that no endogenous peroxidase activity was present in the absence of injected nano sensors (FIGS. 16A and 16C). TEM image analysis of urine from tumour-bearing mice confirmed the presence of AuNCs cleared by the kidney, with size and morphology comparable to as-synthesized AuNCs (FIG. 19C). To ensure that the AuNC-NAv complex was not disassembling in vivo due to poor chemical stability or nonspecific cleavage, a substrate that was not expected to be specifically cleaved in the tumour model was used (Dudani et al., Adv. Funct. Mater. 26, 2919-2928 (2016)). Thrombin-responsive AuNC-P1₂₀-NAv complexes were injected into tumour-bearing and healthy mice and did not result in any significant colorimetric signal in urine from tumour-bearing mice compared to healthy controls (FIG. 5E). This pattern suggested that there is a non-promiscuous release of AuNCs in vivo from AuNC-P2₂₀-NAv complexes that is amplified in tumour-bearing mice, where elevated MMP levels at the site of disease and in circulation may actively disassemble AuNC-NAv complexes. Taken together, these results demonstrate that the AuNC-NAv nanosensors respond to disease-specific proteolytic activity in vivo and enable a direct colorimetric readout of disease state, as evidenced by highly accurate discrimination in a flank tumour model of human colorectal cancer.

See also the figures and figure legends in Loynachan et al., Nat Nanotechnol. 2019 September; 14(9):883-890, which is herein incorporated by reference only for this purpose.

### Materials and Methods

All chemicals were purchased from Sigma-Aldrich unless otherwise stated. Milli-Q water (18.2 MΩ.cm) was used in all the experiments.

### AuNC Synthesis

Synthesis and purification of peptide-capped AuNCs followed published procedures with modifications outlined below (Zhang et al., Sci. Rep. 5, 8669 (2015)). The ratio of protease-cleavable peptide substrate to glutathione was varied in the AuNC synthesis to incorporate functional handles onto the AuNC surface (P1:GSH or P2:GSH, tested at 1:2, 1:4, 1:5, 1:9). Briefly, freshly prepared aqueous solution of gold(III) chloride trihydrate (HAuCl₄, 20 mM, 100 μL) was mixed with 750 μL deionized water in an Eppendorf tube, followed by fast addition of L-Glutathione reduced (GSH, 20 mM) and either peptide P1 or P2 (20 mM) so that final peptide content was fixed at a total volume of 150 μL in varying ratios of P1 or P2:GSH at 25° C. The reaction mixture was heated to 70° C. under gentle stirring (500 rpm) for 24 h. The reaction mixture changed from yellow to colourless within minutes and then turned pale yellow over ca. 12 h, indicating first reduction of Au (III) to Au (I) by the thiol group of the peptides, followed by the reduction of Au(I) thiolate complexes to Au(0) atoms over time assisted by the favourable reduction kinetics at the elevated reaction temperature (Yu et al., ACS Nano 6, 7920-7927 (2012); Luo et al., J. Am. Chem. Soc. 134, 16662-16670 (2012)). After 24 h synthesis, the resulting AuNC solution exhibited both orange luminescence and simultaneous peroxidase-like activity. The AuNCs could be stored at 4° C. for >6 months with negligible changes in optical or catalytic properties. The as-prepared AuNCs were purified through centrifugal ultrafiltration (Amicon Ultra centrifugal filter units Ultra-15, MWCO 10 kDa, Sigma) and buffer exchanged into phosphate buffered saline (PBS, pH 7.2). During ultrafiltration, the AuNCs were collected in the concentrate in the filter device, while any unbound peptide was collected in the filtrate. After purification, AuNCs were resuspended in PBS (20 μM by AuNC particle concentration) and sterile filtered (Millex-GV Filter, Millipore, 0.22 μm). The number of biotinylated ligands per AuNC was calculated by measuring biotin concentration in the filtrate from AuNC purification above, and subsequently subtracting this value from the starting concentration of biotinylated peptide used in the synthesis. Biotin concentration in the filtrate was quantified using the Pierce Biotin Quantitation kit in a 96-well plate following manufacturer's instructions (Thermo Fisher) without any modifications. The molarity of biotin in the sample was calculated using the extinction coefficient for HABA/avidin at 500 nm of 34,000 M⁻¹ cm⁻¹ and path length of 0.5 cm.

### Characterization of Nanoparticles

Dynamic light scattering (DLS, Zeta Sizer Nanoseries, Malvern Instruments, Ltd.) was used to characterize the hydrodynamic diameter of nanoparticles. Absorption measurements were recorded on a SpectraMax M5 multimode microplate reader (Molecular Devices, Ltd.) using SoftMax Pro (Version 5.4) software. For electron microscopy characterization, samples were drop-casted onto carbon-coated copper grids (Electron Microscopy Sciences), and TEM imaging was performed using a JEOL 2100F operating at 200 kV. For preparation of TEM samples, AuNC samples were first desalted (Zeba Spin Desalting Columns, 7K MWCO, Sigma) and 5 μL desalted sample was dropped onto the grid, allowed to incubate for 5 min, and subsequently wicked with filter paper and dried overnight before imaging.

### Evaluation of Peroxidase-Like Activity

The colorimetric readout was carefully optimized to maximize signal intensity from AuNCs by varying the concentration of hydrogen peroxide, pH, and concentration of sodium chloride, and measuring corresponding catalytic activity under these conditions (FIGS. 10A-10D and 10F, Table 7). For stability and catalytic activity of AuNCs in physiological environments, AuNCs (20 μM, 50 μL) were incubated with PBS (50 μL), synthetic urine (Surine Negative Urine Control, Sigma), or fetal bovine serum (FBS, Gibco) for 1 h at 37° C. followed by five-fold dilution in water. For the activity assay, 50 uL of each sample was added to a 96-well plate (Corning, UK) followed by 150 uL chromogenic substrate solution: 1-Step Ultra TMB ELISA Substrate Solution (Thermo Fisher) spiked to a final concentration of 4 M hydrogen peroxide (30% (w/w), Sigma). The absorbance of the reaction solution at 652 nm was monitored up to 25 min after addition of substrate, corresponding to oxidation of TMB by H₂O₂.

For limit of detection (LoD) assays, in a 96-well plate, synthetic urine (25 μL) was mixed with AuNCs (25 μL, varying concentrations, diluted in PBS), 5 M H₂O₂ (100 μL), and 1-Step Ultra TMB ELISA Substrate Solution (100 μL). Absorbance at 652 nm was measured every 20 s for 10 min, and linear regression was used to calculate the slope (A_{652 nm} s⁻¹) over the first 150 sec. LoD was calculated as 3 standard deviations above the mean background signal.

### AuNC-NAv Complex Assembly

In a typical conjugation, 125 μL NeutrAvidin Protein (120 μM, PBS, Thermo Fisher, NAv) was mixed with 1 mL of AuNC-P1 or AuNC-P2 (20 μM) and incubated for 12 h gently shaking (500 rpm) at 37° C. Unbound AuNCs were removed from AuNC-NAv complexes through centrifugal ultrafiltration (Amicon Ultra centrifugal filter units Ultra-15, MWCO 50 kDa, Sigma), where AuNC-NAv complexes remained in concentrate and any unbound AuNCs were collected in the filtrate. After ultrafiltration, AuNC-NAv complexes were resuspended in PBS (30 μM by [AuNC]) and sterile filtered (Millex-GV Filter, Millipore, 0.22 μm).

### In Vivo Renal Clearance Studies

All animal studies were approved by the Massachusetts Institute of Technology (MIT) committee on animal care (MIT protocol 0417-025-20). All animals received humane care, and all experiments were conducted in compliance with institutional and national guidelines. GSH-templated and substrate functionalized AuNCs were diluted to 10 μM [AuNC] in sterile PBS. Female Swiss Webster mice (4-6 weeks old, Taconic) were intravenously administered 2000 pmol AuNCs via the tail vein (10 μM [AuNC], 200 μL). The injected dose of glutathione-templated gold nanoclusters ranged from 1.6 to 2.4 mg kg⁻¹ in terms of gold content, which is well below the maximal tolerated dose reported for both mice and non-human primates (1059 mg kg⁻¹ by GSH-AuNC content, ˜530 mg kg⁻¹ by gold content) (Kwong et al., Chemie Int. Ed. 57, 266-271 (2018)).

After nanocluster injection, urine was collected at the indicated timepoints for catalytic activity assay and ICP-MS measurements. Mice were placed in custom housing with a 96-well plate base for urine collection. After 1 h, their bladders were voided, and collected urine volume was measured. Clearance of active AuNCs was quantified via catalytic activity assay, and urine gold content was quantified by ICP-MS. Catalytic activity and gold content measurements of the collected urine samples were compared to that of the injected dose and normalized using urine volumes. Urine concentration may be dependent on many host and environmental factors, and therefore normalization between urine samples is required. In this study, urine volume and injected dose were used for normalization. Alternatively, co-administered free reporters that pass into urine independent of disease state, such as glutamate fibrinopeptide B (Kwong et al., Nat. Biotechnol. 31, 63-70 (2013)) or inulin (Warren et al., J. Am. Chem. Soc. 136, 13709-13714 (2014)), could also be measured in urine and used to normalize the level of AuNCs released by protease activity. Pearson's correlation coefficient (r) was computed to assess the relationship between renal clearance as measured by catalytic activity assay or ICP-MS (gold content).

### Urine Catalytic Activity Assays

For all assays, 25 μL of urine was diluted into 25 μL PBS in a transparent 96-well plate and allowed to equilibrate at room temperature for 15 min. 100 μL each of 5 M H₂O₂ (Sigma) and TMB (Thermo Fisher) were then added, and the plate was read kinetically at 652 nm over the course of 30 min. In all catalytic activity assays involving AuNCs in collected urine, the final pH was acidic due to the acidic pH of the TMB and hydrogen peroxide substrate mix resulting in a final reaction pH<4. For renal clearance studies, the concentration of active AuNCs present in the urine was quantified via reference to a calibration curve of known AuNC concentrations. For disease detection studies, the initial reaction velocity was quantified as the rate of change of the absorbance at 652 nm over the first 10 min of the reaction (A652 min⁻¹). Initial velocity analysis was preferred over analysis of a single time point measurement of absorbance at 652 nm, as urine collected from different mice had varying degrees of background levels of absorbance at this wavelength based on the hydration state of each mouse. This variable background was removed in the initial velocity analysis as the background absorbance from initial coloration of urine was constant over time. Limit of detection was calculated to be the lowest concentration of the linear portion of the calibration curve (measured as the initial velocity of catalytic activity of relevant AuNC batch).

### Inductively Coupled Plasma-Mass Spectrometry (ICP-MS) on Urine Samples

Urine samples were digested in aqua regia (TraceMetal Grade hydrochloric acid, Fisher Chemical; ARISTAR ULTRA nitric acid, VWR) for 24 h. The digested samples were further diluted into an ICP-MS matrix composed of 4% HC1 / 4% HNO₃. The gold content in each sample was measured using an Agilent 7900 ICP-MS using an indium internal standard (5 ppb; TraceCERT, Sigma) and gold standard (Inorganic Ventures) for the calibration curve prepared in the ICP-MS matrix.

### Cell Culture

For xenograft studies, LS174T (ATCC CL-188) cells were cultured in Eagle's Minimal Essential Medium (EMEM, ATCC) supplemented with 10% (v/v) FBS (Gibco) and 1% (v/v) penicillin-streptomycin (CellGro). For in vitro cytotoxicity assays, HEK293T (ATCC CRL-3216) cells were cultured in Dulbecco's Modified Eagle Medium (DMEM, ATCC) supplemented with 10% (v/v) FBS (Gibco) and 1% (v/v) penicillin-streptomycin (CellGro). Cells were passaged when confluence reached 80%.

### In Vitro Cytotoxicity Studies

For in vitro cytotoxicity studies, HEK293T cells were plated in a 96-well plate (10,000 cells per well) and allowed to adhere to the wells. 24 h post seeding, cells were incubated with varying concentrations of AuNC-NAv complex (diluted in PBS) for 24 h. Cell viability was evaluated using the MTS (3-(4,5-dimethylthiazol-2-yl)-5-(3-carboxymethoxyphenyl)-2-(4-sulfophenyl)-2H-tetrazolium) assay (Promega).

### In Vivo Toxicity Studies

AuNC-NAv complex (AuNC-P1₂₀-NAv or AuNC-P2₂₀-NAv, 15 μM [AuNC], 200 μL ˜3000 pmol) was intravenously injected into immunocompetent female Swiss Webster mice (4-6 weeks old, Taconic). The mass of each mouse was monitored for 4 weeks p.i. and compared with masses of PBS injected control mice. Heart, lung, liver, spleen, and kidney tissues were collected from the mice at 1 h, 24 h, or 10 days p.i., fixed in 10 wt % formalin, paraffin embedded, stained with haematoxylin and eosin, and then examined by a veterinary pathologist and compared to organs from PBS injected control mice.

### Pharmacokinetic Studies

To analyse the blood half-life of the AuNC-NAv complex, female Swiss Webster mice (4-6 weeks old, Taconic) were injected with AuNC-P2₂₀-NAv (15 μM [AuNC], 200 μL˜3000 pmol) labelled with the photostable near-IR dye Alexa Fluor 750 Succinimidyl Ester (Invitrogen). Blood was withdrawn retro-orbitally (˜70 μL) and then immediately transferred into 70 μL of PBS with 5 mM EDTA and centrifuged to pellet blood cells. Concentration of the AuNC-NAv complex in plasma was measured using an Odyssey CLx infrared scanner (Li-Cor Inc.).

For biodistribution studies in healthy animals, female Swiss Webster mice (4-6 weeks old, Taconic) were injected with either near-IR dye labelled AuNCs (10 μM [AuNC], 200 μL ˜2000 pmol) or AuNC-P2₂₀-NAv (15 μM [AuNC], 200 μL˜3000 pmol) complexes. Mice were sacrificed at 1 h, 3 h, 24 h, 1 week, or 4 weeks p.i., and organ and tumour accumulation was measured using an Odyssey CLx scanner (Li-Cor Inc.) and quantified using ImageStudio

(Version 5.2, Li-Cor Inc.). Organ accumulation was quantified as signal intensity per unit area, calculated for each organ as the difference between the experimental group (near-IR dye labelled AuNCs or AuNC-P2₂₀-NAv) versus the PBS-injected control. Values were scaled by a constant factor for all time points within each treatment group (near-IR dye labelled AuNCs or AuNC-P2₂₀-NAv) to fall within the range shown. For mice injected with free AuNCs, urine was also collected at the indicated time points and analysed by both ICP-MS (for gold content analysis) and catalytic activity assay.

For biodistribution studies in tumour-bearing mice, nude mice bearing LS174T flank tumours were infused with either near-IR dye labelled neutravidin carrier (VivoTag750, PerkinElmer; 1 μM by VT750), MMP-cleavable AuNC-P2₂₀-NAv complex (15 μM [AuNC], 200 μL˜3000 pmol, Alexa Fluor 750), or free AuNCs (10 μM [AuNC], 200 μL˜2000 pmol, Alexa Fluor 750). Mice were sacrificed 1 h p.i., and organ and tumour accumulation were measured using an Odyssey CLx scanner (Li-Cor Inc.) and quantified using ImageStudio (Version 5.2, Li-Cor Inc.). Organ accumulation was quantified as signal intensity per unit area, calculated for each organ as the difference between the experimental group (fluorescently labelled carrier, complex, or free nanocluster) versus the PBS-injected control, and scaled to fall within the range shown.

### Colorectal Cancer Xenograft Studies

Female NCr Nude mice (4-5 weeks, Taconic) were injected bilaterally with 3×10⁶ LS174T cells per flank. Two weeks after inoculation, tumour-bearing mice and age-matched controls were injected with either 15 μM MMP-sensitive or thrombin-sensitive (control) AuNC nanosensors in 200 μL of PBS (concentrations determined by [AuNC]). After nanosensor injection, mice were placed in custom housing with a 96-well plate base for urine collection. Based on the measured blood half-life of the AuNC-NAv complex, the degree of tumour accumulation 1 h p.i., as well as the results from the FCS cleavage assays (80% of AuNCs cleaved from complex within 1 h), 1 h p.i. was selected as the time point for urine collection (Kwong et al., Nat. Biotechnol. 31, 63-70 (2013); Warren et al., Proc. Natl. Acad. Sci. U.S.A. 111, 3671-6 (2014); Kwon et al., Nat. Biomed. Eng. 1, 0054 (2017)). After 1 h, bladders of the mice were voided to collect between 100-200 μL of urine. Urine was analysed via the catalytic activity measurements described above.

### Statistical Analyses

All statistical analyses were conducted in GraphPad 7.0 (Prism). All sample sizes and statistical tests are specified in figure legends. The D'Agostino-Pearson test was used to assess normality and thus determined the statistical test used. For each animal experiment, groups were established before tumorigenesis or treatment with AuNC-PX, and therefore no randomization was used in the allocation of groups. Investigators were not blinded to the groups and treatments during the experiments.

### Solid Phase Peptide Synthesis

Peptides were synthesized manually on Rink amide resin using standard fluorenyl methoxycarbonyl (Fmoc) chemistry. The Fmoc protecting group was removed from the resin by incubating with piperidine/DMF (20:80) for 2×10 min. Fmoc-protected amino acids were activated with 4 molar equivalents of the Fmoc protected amino acids, 3.95 molar equivalents of N,N,N′,N′-Tetramethyl-O-(1H-benzotriazol-1-yl)uronium hexafluorophosphate, and 6 molar equivalents of diisopropylethylamine in DMF. The coupling solution was added to the resin and the coupling reaction was allowed to proceed for 3 h. Peptides were cleaved in trifluoroacetic acid/triisopropylsilane/H₂O (95:2.5:2.5) containing DTT for 4 h. The solvent was removed in vacuum and the peptide was precipitated in cold ether. The crude products were further purified using reversed phase preparative high-performance liquid chromatography (Shimadzu) in an acetonitrile/water gradient under acidic conditions on a Phenomenex C18 Gemini NX column (5 micron pore size, a 110 Å particle size, 150×21.2 mm). Molecular weight of peptides was verified using liquid chromatography-mass spectrometry (LC-MS, Agilent Technologies).

### In Vitro Gel Filtration Chromatography (GFC) Assays

AuNC-NAv complexes (10 μM) were first incubated with a recombinant enzyme: MMP9 (Active, Human, Recombinant, PF140, Merck Millipore); MMPI (Active, Human, Recombinant, E. coli, 444270, Merck Millipore); MMP13 (Active, Human, Recombinant, 444287); or thrombin from human plasma (T7009, Sigma, 100 U·mL⁻¹ in a 0.1% (w/v) bovine serum albumin solution). Enzyme and AuNC-NAv were incubated at 37° C. gently shaking (500 rpm). Incubation times varied (1-12 h) and concentration of enzyme was fixed at 50 nM, where the final peptide substrate concentration was maintained at >1000 molar excess to enzyme concentration.

Three identical glass chromatography columns were packed with Sephacryl S200 high resolution resin (column D: 1 cm, H: 18 cm, resin: GE Healthcare Life Sciences, fractionation range for globular proteins 5-250 kDa) to separate samples based on size. Columns were thoroughly cleaned between experiments with PBS. In a typical GFC experiment, ca. 200 μL of 10 μM AuNC-PX, AuNC-PX-NAv, and AuNC-PX-NAv+50 nM enzyme (after incubation) were loaded onto each column in parallel. As soon as the sample was added to the resin bed, 24, 500 μL fractions were collected into individual Eppendorf tubes, while PBS was added to the column reservoir. After fractions were collected, a catalytic activity assay was performed on the samples. For the activity assay, 100 μL of each fraction was added to a 96-well plate, followed by 100 μL substrate solution (1-Step Ultra TMB ELISA Substrate Solution with 4 M H₂O₂). The absorbance of the reaction solution at 652 nm was monitored up to 30 min after addition of substrate, corresponding to oxidation of TMB by H₂O₂. The composition of the sample could be determined based on how quickly it eluted from the column as measured by activity. Larger AuNC-NAv complexes eluted within the first 7 mL, and smaller bare AuNCs eluted more slowly and were found in fractions 7-12 mL, corroborated by DLS of column fractions. Absorbance at a fixed time point was plotted as a function of eluted volume, where clear peaks in absorbance were associated with either AuNC-NAv complexes or bare AuNCs. For AuNC-NAv complexes incubated with enzymes, the proportion of liberated AuNCs could be measured by calculating the area under the curve corresponding to 7-12 mL eluted volume (fractions corresponding to bare AuNCs) compared to the total area for the entire eluted volume.

### Fluorescence Correlation Spectroscopy (FCS)

FCS is an autocorrelation analysis of temporal fluctuations of fluorescence intensity due to diffusion of fluorescent particles in and out of a small observation volume, useful for monitoring binding or cleavage events by analysing changes in diffusivity over time. For FCS analysis, AuNC batches were labelled with Oregon Green fluorescent dye (at the free amino group of GSH) and assembled into complexes with the neutravidin core. AuNC-P1 and AuNC-P2 were labelled with 50 molar excess reactive dye (Oregon Green 488 Carboxylic Acid, Succinimidyl Ester, 6-isomer, Thermo Fisher), further called AuNC-PX-OG. Unreacted dye was removed using Zeba Spin Desalting Columns 7K MWCO (Thermo Fisher). AuNC-PX-OG-NAv complexes were assembled following the “AuNC-NAv complex assembly” protocol outlined in the online Methods section, and purified to remove unbound AuNC-PX-OG. AuNC-PX-OG-NAv complexes were further incubated with enzymes, and kinetics of AuNC-NAv complex disassembly via substrate cleavage was monitored over time using FCS.

### Sample Preparation for Measuring Enzyme Cleavage Kinetics

For MMP9: 0.33 μL MMP9 stock (Merck PF140 lot#2872521, 0.1 mg·mL⁻¹˜1500 nM, 57.28 Units/h/μg P) was added per 10 μL sample stock (20 μM, AuNC), for a final enzyme concentration of 50 nM, with AuNCs in 400 molar excess to MMP9. Since AuNCs bear ca. 20 peptide substrates per particle, there was ca. 8000 molar excess peptide substrates per enzyme. For thrombin: 0.58 μL thrombin stock (100 U·mL⁻¹, 32 μg·mL⁻¹-860 nM) was added per 10 μL sample stock (20 μM, AuNC), for a final enzyme concentration of 50 nM, with AuNCs in 400 molar excess to thrombin. All enzyme incubations were performed at 37° C., and incubations longer than 3 h were maintained at 37° C. while shaking (300 rpm). Samples were then diluted in pre-warmed PBS for FCS measurements.

### FCS Measurements

In the measurement, labelled particles diffuse through the detection volume, producing a fluctuating fluorescence signal which is subjected to an autocorrelation algorithm yielding an autocorrelation curve, G (τ), which shows the mobility of the particles. The diffusion time of the particles, τ_{D}, can be estimated from the inflection of the decay of the autocorrelation curve.

FCS was performed on a commercial LSM 880 (Carl Zeiss, Jena, Germany) equipped with an incubation chamber. All measurements were performed at 37° C. An Ar⁺ laser was used as excitation source for the 488 nm wavelength. Appropriate filter sets were used to detect the fluorescence signal (LP 505). The laser beam passed through a 40× C-Apochromat water immersion objective with a numeric aperture of 1.2 to focus the beam into the sample droplet. Measurements were performed 200 μm above the ibidi 8-well bottom plate (80826, ibidi, Germany) using a 5 μL droplet of sample for each condition. OregonGreen 488 carboxylic acid in PBS (OG488, 06149, ThermoFisher Scientific, NHS-ester was first deactivated by overnight incubation in PBS at room temperature) was used as a standard to calibrate the beam waist (D=4.1×10⁻⁶ cm²/s at 25° C., and when corrected for the higher temperature used: D=5.49×10⁻⁶ cm²/s at 37° C.) (Kapusta, PicoQuant GmbH Appl. Note (2010)). Immediately before the measurement, stocks or incubated samples were diluted 100-fold in pre-warmed PBS and 5 μL was placed into the measuring chamber. The sample was equilibrated and bleached for 5×5 s and 25×5 s intensity traces were recorded, autocorrelated and analysed for each sample. Autocorrelation curves were created in ZEN software (Carl Zeiss, Jena, Germany) and the curves were exported for further analysis using PyCorrfit program 1.1.1. (Muller et al., Bioinformatics 30, 2532-2533 (2014)). For all the graphs, data for the 25 curves are given except for the autocorrelation curves, which are always the average curve for the whole measurement (125 s).To calculate the percentage of AuNCs cleaved from the AuNC-NAv complex, stocks of clusters/complexes were first fitted using one component fits (G_{1comp}(τ)) to obtain the diffusion times for the pure components. Second, samples incubated with enzymes were fitted with two component fits (G_{2comp}(τ)) with one component fixed to pure cluster diffusion (τ₁) and the other fixed to pure complex diffusion (τ₂) to yield the fraction of free clusters (F₁), which is equivalent to the fraction cleaved. A triplet fraction with a triplet time of 10 μs was included for all the curves.

$$\begin{matrix}{G}_{1 comp} ( τ ) = ( 1 + \frac{T}{1 - T} {e}^{\frac{- τ}{{τ}_{trip}}} ) * \frac{1}{N * ( 1 + \frac{τ}{{τ}_{D}} ) * \sqrt{1 + \frac{τ}{{SP}^{2} {τ}_{D}}}} & Equation 1 \\ {G}_{2 comp} = ( 1 + \frac{T}{1 - T} {e}^{\frac{- τ}{{τ}_{trip}}} ) * \frac{1}{N} * [ \frac{{F}_{1}}{( 1 + \frac{τ}{{τ}_{1}} ) * \sqrt{1 + \frac{τ}{{SP}^{2} {τ}_{1}}}} + \frac{1 - {F}_{1}}{( 1 + \frac{τ}{{τ}_{2}} ) * \sqrt{1 + \frac{τ}{{SP}^{2} {τ}_{2}}}} ] & Equation 2\end{matrix}$$

T is the triplet fraction with corresponding triplet time τₜᵣᵢₚ, N is the effective number of diffusing species in the confocal volume (N=n1+n2), τ_{D} is the diffusion time (τ₁, τ₂ diffusion times of corresponding fractions), F₁ fraction of component with diffusion time τ₁, and SP is the structural parameter describing the ratio of height to width of the confocal volume (fixed to 5). The following equation relates the x-y dimension of the confocal volume (ω²_{xy}), which was calibrated by a standard measurement of OG488 in PBS, to the diffusion coefficient (D), which was calculated for each sample using the obtained diffusion time (τ_{D}):

$$D = \frac{{ω}_{xy}^{2}}{4 {τ}_{D}} \qquad Equation 3$$

Stokes-Einstein equation was used to calculate hydrodynamic diameter via the obtained diffusion coefficients. For the AuNC-P2₁₃-NAv complex, the percentage of cleaved AuNCs with time was linear over the first 500 min. of MMP9 incubation, whereas for AuNC-P2₂₀-NAv the percentage cleaved was linear over just the first 16 min. of enzyme incubation. The linear regions were analysed by linear regression, and the rates of cleavage were calculated.

In vitro cleavage assays with quenched substrates P1Q (1 μM by peptide) was incubated with recombinant mouse thrombin (12.5 nM working concentration; Haematologic Technologies) in a 384-well plate at 37° C. in PBS-BSA (0.1% w/v). P2Q (1 μM by peptide) was incubated with recombinant human MMP-9 (100 nM working concentration; Enzo Life Sciences) in activity buffer (50 mM Tris, 150 mM NaCl, 5 mM CaCl2, 1 μM ZnCl₂) containing 0.1 wt % BSA. Fluorescence dequenching was monitored at 37° C. using a Tecan Infinite 200pro microplate reader (Tecan).

### Example 9

### Liposome Encapsulated Nanocatalysts for Sensing of Disease-Associated Enzymes

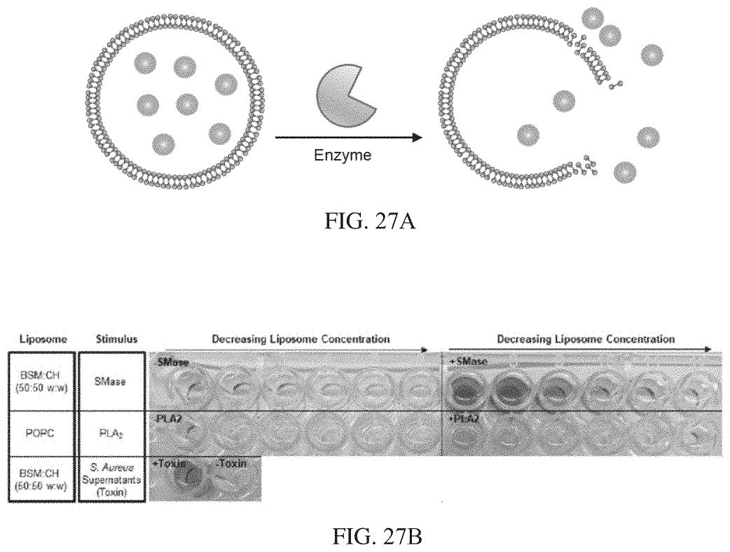

To develop a liposome-based sensor of disease-associated enzymes, liposomes that encapsulated nanocatalysts in an aqueous core were developed. As shown in FIG. 27A, the liposomes were engineered such that they ruptured upon exposure to a disease-associated enzyme. Liposomes formulated with brain sphingomyelin:cholesterol (BSM:CH, 50:50 w:w) or phosphatidylcholine (POPC) were created. Then, it was determined whether the sensors could be used to detect the presence of an environmental trigger. As shown in FIG. 27B, liposomes comprising BSM:CH (50:50 w:w) released nanocatalysts in the presence of SMase and S. Aureus supernatants. Liposomes comprising POPC released nanocatalysts in the presence of PLA2 (FIG. 27B). Therefore, liposome-encapsulated nanocatalysts could be used to sense disease-associated enzymes.

What is claimed is:

1. An in vivo or in vitro sensor comprising a scaffold comprising an environmentally-responsive linker that is attached to a nanocatalyst, wherein the nanocatalyst is capable of being released from the scaffold when exposed to an environmental trigger, and optionally wherein the sensor is formulated for in vivo delivery, optionally wherein the environmental trigger is an enzyme.

2. The sensor of claim 1, wherein the scaffold encapsulates a nanocatalyst, optionally wherein the scaffold is a liposome, polymersome, or a PLGA nanoparticle.

3. The sensor of any one of claims 1-2, wherein the nanocatalyst is a catalytic nanocluster or a nanocatalyst, optionally, wherein the catalytic nanocluster is a transition metal nanocluster selected from the group consisting of a platinum nanocluster, a silver nanocluster, and a gold nanocluster and optionally, wherein the nanocatalyst is selected from the group consisting of an iron oxide nanoparticle and an iridium nanoparticle.

4. The sensor of any one of claims 1-3, wherein the environmentally-responsive linker is temperature-responsive, pH-responsive, or an enzyme-specific substrate.

5. The sensor of any one of claims 1-4, wherein the nanocatalyst is less than 5 nm in size, optionally less than 2 nm in size.

6. The sensor of any one of claims 1-5, wherein the scaffold is greater than about 5 nm in diameter.

7. The sensor of any one of claims 1-6, wherein the scaffold comprises a protein, a polymer, or a nanoparticle.

8. The sensor of claim 7, wherein the protein comprises avidin.

9. The sensor of claim 8, wherein the avidin is selected from the group consisting of avidin, streptavidin, NeutrAvidin, and CaptAvidin.

10. The sensor of any one of claims 1-9, wherein the environmentally-responsive linker is further attached to a functional handle and wherein the environmentally-responsive linker is located between the functional handle and the nanocatalyst.

11. The sensor of claim 10, wherein the functional handle is selected from the group consisting of a dibenzocyclooctyne (DBCO), an amine, a SpyCatcher tag, a SpyTag, a biotin, avidin, an alkyne, and an azide.

12. The sensor of claim 10 or 11, wherein the functional handle is linked to the scaffold.

13. The sensor of any one of claims 1-12, the nanocatalyst is luminescent.

14. The sensor of any one of claims 1-13, wherein the nanocatalyst is capable of disproportionating H₂O₂.

15. The sensor of claim 14, wherein the nanocatalyst is capable of disproportionating H₂O₂ in physiological environments.

16. The sensor of any one of claims 1-15, wherein the nanocatalyst comprises a zwitterionic peptide capping layer.

17. The sensor of any one of claims 4-16, wherein the enzyme-specific substrate is a disease-specific substrate.

18. The sensor of claim 17, wherein the disease is cancer, HIV, malaria, an infection or pulmonary embolism.

19. The sensor of any one of claims 1-18, wherein the sensor comprises a single environmentally-responsive linker, a single nanocatalyst, or a combination thereof.

20. The sensor of any one of claims 1-19, wherein the sensor comprises multiple environmentally-responsive linkers, multiple nanoclusters, or a combination thereof.

21. The sensor of any one of claims 1-20, wherein the ratio of the number of environmentally-responsive linkers to the number of nanocatalysts is at least 1, optionally wherein the ratio is between 1 and 20.

22. The sensor of any one of claims 1-21, wherein the surface area to volume ratio of the nanocatalyst is about 1.2 to about 6.

23. A method comprising:
(a) administering to a subject a sensor, wherein the sensor comprises a scaffold comprising an environmentally-responsive linker that is attached to a nanocatalyst, wherein the nanocatalyst is capable of being released from the scaffold when exposed to an environmental trigger in vivo or in vitro, optionally wherein the subject is a human subject; and
(b) detecting in a biological sample obtained from the subject the nanocatalyst, wherein detection of the nanocatalyst in the biological sample is indicative of the environmental trigger being present within the subject.

24. The sensor of claim 23, wherein the nanocatalyst is a transition metal nanocluster, optionally, wherein the transition metal nanocluster is a platinum nanocluster, a silver nanocluster, or a gold nanocluster and optionally, wherein the nanocatalyst is an iron oxide nanoparticle, or an iridium nanoparticle,

25. The sensor of any one of claims 23-24, wherein the environmentally-responsive linker is an enzyme-specific substrate, wherein the environmental trigger is the enzyme and wherein the detection of the nanocatalyst is indicative of the enzyme being in an active form within the subject.

26. The method of any one of claims claim 23-25, wherein the biological sample is not derived from the site of exposure to the environmental trigger, optionally wherein the sample is a urine sample, blood sample, or tissue sample.

27. The method of any one of claims 23-26, wherein the detecting comprises a colorimetric assay, luminescence, or fluorescence assay.

28. The method of any one of claims 23-27, wherein the detection comprises detecting the catalytic activity of the nanocatalyst.

29. The method of claim 28, wherein the detecting comprises an oxidation assay with a peroxidase substrate and detection of the oxidized substrate, optionally, wherein the peroxidase substrate is a chromogenic substrate.

30. The method of any one of claims 25-29, wherein the enzyme-specific substrate is a disease-specific substrate.

31. The method of claim 30, further comprising diagnosing the subject with the disease based on the detection of the nanocatalyst in the biological sample.

32. The method of claim 31, wherein the disease is selected from the group consisting of cancer, HIV, malaria, an infection, and pulmonary embolism.

33. A method comprising incubating an environmentally-responsive linker and a reducing agent with chloroauric acid (HAuCl₄), wherein the environmentally-responsive linker comprises a cysteine residue or is thiol-terminated and optionally, wherein the resulting gold nanoclusters are capped and stabilized by both the reducing agent and an environmentally-responsive linker and exhibit both intrinsic fluorescence and peroxidase-like catalytic activity, and wherein the gold nanocluster is capable of being released from the environmentally-responsive linker in vivo, and optionally wherein the nanocluster synthesis proceeds at an elevated temperature of at least 70° C. for more than 12 hrs and optionally wherein the reducing agent is L-glutathione (GSH) peptide.

34. The method of 33, wherein the environmentally-responsive linker further comprises a functional handle.

35. The method of 34, wherein the functional handle is selected from the group consisting of a dibenzocyclooctyne (DBCO), an amine, a SpyCatcher tag, a SpyTag, a biotin, avidin, an alkyne, and an azide.

36. The method any one of claims 34-35, further comprising incubating the environmentally-responsive linker attached to the nanocatalyst with a scaffold comprising a cognate functional handle partner, optionally wherein the cognate functional handle partner is selected from the group consisting of a dibenzocyclooctyne (DBCO), an amine, a SpyCatcher tag, a SpyTag, a biotin, an alkyne, and an azide.

37. The method of 36, the avidin is selected from the group consisting of avidin, streptavidin, NeutrAvidin, and CaptAvidin.

38. The method of any one of claims 33-37, wherein the gold nanocluster has a surface area to volume ratio of the gold nanocluster is about 1.2 to about 6.

39. An in vivo or in vitro sensor comprising a scaffold that encapsulates a nanocatalyst, wherein the nanocatalyst is capable of being released from the scaffold when exposed to an environmental trigger, and optionally wherein the sensor is formulated for in vivo delivery, optionally wherein the environmental trigger is an enzyme.

40. The sensor of claim 39, wherein the scaffold is a liposome that comprises brain sphingomyelin (BSM) and cholesterol (CH).

41. The sensor of claim 39, wherein the scaffold is a liposome that comprises phosphatidylcholine (POPC).

42. The sensor of any one of claims 39-41, wherein the environmental trigger is a phospholipase A2 (PLA₂) enzyme, sphingomyelinase (SMase), and/or a toxin.

43. The sensor of claim 42, wherein the toxin is alpha-hemolysin.

44. A method comprising:
(a) administering to a subject the sensor of any one of claims 39-43, wherein the sensor comprises a scaffold that encapsulates a nanocatalyst, wherein the nanocatalyst is capable of being released from the scaffold when exposed to an environmental trigger in vivo or in vitro, optionally wherein the subject is a human subject; and
(b) detecting in a biological sample obtained from the subject the nanocatalyst, wherein detection of the nanocatalyst in the biological sample is indicative of the environmental trigger being present within the subject.


SEQUENCE LISTING (6 sequences)

SEQ ID NO: 1 - protein, 13 aa, Artificial Sequence
  Synthetic Polypeptide
  MISC_FEATURE (4)..(4): d-amino acid
  MISC_FEATURE (6)..(7): scissile bond
SGGFPRSGGSGGC

SEQ ID NO: 2 - protein, 20 aa, Artificial Sequence
  Synthetic Polypeptide
  MISC_FEATURE (11)..(11): d-amino acid
  MISC_FEATURE (13)..(14): scissile bond
GGGSGGGSGGFPRSGGGGGC

SEQ ID NO: 3 - protein, 13 aa, Artificial Sequence
  Synthetic Polypeptide
  MISC_FEATURE (6)..(7): scissile bond
GGGPLGVRGKGGC

SEQ ID NO: 4 - protein, 20 aa, Artificial Sequence
  Synthetic Polypeptide
  MISC_FEATURE (13)..(14): scissile bond
GGGGGGGGGGPLGVRGKGGC

SEQ ID NO: 5 - protein, 11 aa, Artificial Sequence
  Synthetic Polypeptide
  MISC_FEATURE (3)..(3): d-amino acid
  MISC_FEATURE (5)..(6): scissile bond
  MISC_FEATURE (10)..(10): modified by CPQ2-PEG2
GGFPRSGGGKC

SEQ ID NO: 6 - protein, 11 aa, Artificial Sequence
  Synthetic Polypeptide
  MISC_FEATURE (5)..(6): scissile bond
  MISC_FEATURE (10)..(10): modified by CPQ2-PEG2
GGPLGVRGKKC